(19) United States
(12) Patent Application Publication
(10) Pub. No.: US 2026/0114980 A1
(43) Pub. Date: Apr. 30, 2026

(54) FILTER FOR USE WITH A MEDICAL DEVICE

(71) Applicant: MiRus LLC, Marietta, GA (US)

(72) Inventors: Jay Yadav, Atlanta, GA (US); Jorge Jimenez, Marietta, GA (US); Chris Hayden, Marietta, FL (US); Elias Rodriguez, Marietta, GA (US); Miheer Joshi, Marietta, GA (US); Jason White, Atlanta, GA (US); Ken Holloway, Marietta, GA (US)

(21) Appl. No.: 19/366,609

(22) Filed: Oct. 23, 2025

Related U.S. Application Data
(60) Provisional application No. 63/712,489, filed on Oct. 27, 2024.
(60) Provisional application No. 63/712,811, filed on Oct. 28, 2024.

(51) Int. Cl. A61F 2/01; A61F 2/24; B22F 3/24; B22F 5/00

(52) U.S. Cl. CPC A61F 2/013; A61F 2/0105; A61F 2/011; A61F 2/2433; A61F 2210/0014; A61F 2230/0067; A61F 2240/001; B22F 3/24; B22F 2003/241; B22F 5/00

(57) ABSTRACT

A medical device delivery arrangement that includes a medical device, a delivery system, and a filter arrangement. The delivery system includes a catheter and a guide shaft that are configured to move independently from one another. The filter arrangement is configured to move between a constrained-collapsed position and a deployed position. The filter arrangement includes a filter frame and a filter material. The guide shaft is moveable relative to the filter arrangement between a constraining and non-constraining position. The filter arrangement is positioned in the constrained-collapsed position when the guide shaft is in the constraining position due to the guide shaft being positioned partially or fully over the filter arrangement. The filter arrangement is in the deployed position when the guide shaft is in the non-constraining position due the guide shaft being positioned distal to the filter arrangement.

### REFERENCED APPLICATIONS

The present disclosure claim priority on U.S. Provisional Application Ser. Nos. 63/712,811 filed Oct. 28, 2024 and 63/712,489 filed Oct. 27, 2024, both of which are incorporated fully herein by reference.

### FIELD OF DISCLOSURE

The present disclosure relates generally to medical devices and medical device applications, and particularly to a vascular medical device, and more particularly to a vascular medical device that is used with a filter arrangement that will at least partially capture dislodged material in fluid passing through the filter arrangement that is caused by the deployment of the medical device. The filter arrangement is configured to inhibit or prevent dislodged emboli resulting at least partially from the expansion of the prosthetic heart valve from entering one or more arteries that are located rearwardly of the expanded prosthetic heart valve.

### BACKGROUND OF DISCLOSURE

Medical devices such as Transcatheter aortic replacement valves (TAVR valves) represent a significant advancement in prosthetic heart valve technology. TAVR valves bring the benefit of heart valve replacement to patients that would otherwise not be operated on. Transcatheter aortic valve replacement (TAVR) can be used to treat aortic valve stenosis in patients who are classified as high-risk for open heart surgical aortic valve replacement (SAVR). Non-limiting TAVR valves are disclosed in U.S. Pat. Nos. 5,411,522; 6,730,118; 10,729,543; 10,820,993; 10,856,970; 10,869,761; 10,952,852; 10,980,632; 10,980,633; 12,383,399 and US Pub. No. 2020/0405482, all of which are incorporated fully herein by reference. The frame material used to form the TAVR valve is typically TiAlV alloy, CoCr alloy or Nitinol™. The vast majority of cardiovascular implants include valves that are made at least in part using a CoCr alloy or Nitinol materials for construction of the structural frame of the valve.

A TAVR valve is designed to be compressed into a small diameter catheter, remotely placed within a patient's diseased aortic valve to take over the function of the native valve. Some TAVR valves are balloon-expandable, while others are self-expandable. In both cases, the TAVR valve is deployed within a calcified native valve that is forced permanently open and becomes the surface against which the frame is held in place by friction. A prosthetic heart valve can also be used to replace failing bioprosthetic or transcatheter valves, commonly known as a valve in valve procedure. Major TAVR advantages to the traditional surgical approaches include refraining from cardiopulmonary bypass, aortic cross-clamping and sternotomy which significantly reduces patients'morbidity.

However, there are several complications that are associated with current TAVR valves. For example, stroke is a significant adverse event related to TAVR procedures wherein 2-4% of TAVR cases report perioperative stroke. (See FIG. 21). Ischemic stroke, which is caused by emboli becoming dislodged during the TAVR procedure and which travels to the arteries of the brain can create blockages, which blockages are common causes of stroke. The embolic material that is calcified about the aortic valve can be dislodged from the aortic wall during a TAVR procedure from the expansion of the TAVR in the heart. Studies have presented data that patients undergoing a TAVR procedure had an increased incidence of stroke if not using embolic protection. (Proposed Standardized Neurological Endpoints for Cardiovascular Clinical Trials, J Am Coll Cardiol 2017; 69:679-91; Cerebral Embolic Protection during Transcatheter Aortic-Valve Replacement, N Engl J Med 2022; 387:1253-63; Acute Brain Infarctions and Periprocedural Stroke, Journal of the American College of Cardiology: Vol. 8, No. 8, 2024:723-725).

One prior art filter device, the Sentinel™M device offered by Bostin Scientific, has been used to capture embolic material that has dislodged from the heart during a TAVR procedure. The Sentinel™ device includes two embolic protection filters that are delivered via endovascular techniques via the right radial approach. One of the embolic filters is placed in the right subclavian vessel and the other is placed in the left carotid artery. One limitation of the Sentinel™ device is that it only protects the right hemisphere and left anterior circulation to the patient's brain, with no protection of the left posterior circulation of the brain or any other arteries of the patient (kidneys, liver, legs, etc.). Also, the Sentinel™ device requires additional vessel access (e.g., radial access) which increases the potential for vascular access complications and can interfere with the TAVR valve itself as the TAVR valves crosses the aortic arch.

Other embolic filters that have been utilized in other cardiovascular applications, but not in TAVR procedures, include the Abbott Emboshield™ NAV, Johnson & Johnson Angioguard™, TriGUARD3™, ProtEmbo™, Emblok™, Embolizer™, POINT-GUARD™ CAPTIS™, FLOWer™, and the Contego Medical Paladin™ and Vanguard™ systems. (See FIG. 20).

Various limitations of prior art filters are a) the filter is not part of TAVR delivery system, thus requires additional vascular access and procedural steps to use the filter, b) the filter is required to be inserted into the heart prior to use of the TAVR delivery system inserting the TAVR to the heart, thus requiring the filter having long aortic arch dwell times, thereby increasing risk of thrombosis and iatrogenic emboli from scraping of aortic arch or carotids, c) some of the filters are only configured to protecting cerebral arteries, thus allowing emboli particles to pass into unprotected arteries and potentially damage other critical organs such as kidneys, intestines, and extremities, and d) some of the filters do not capture emboli, but only deflects the emboli away from certain, but not all, of the arteries, thus allowing emboli particles to pass into unprotected arteries and potentially damage other critical organs such as kidneys, intestines, and extremities. (See FIG. 20).

In view of the current state of the art of prosthetic heart valves, there is a need for a filter arrangement that includes one or more of the following features: a) the filter arrangement captures, not merely deflects, embolic material that has dislodged from the heart during a prosthetic heart valve procedure, b) the filter arrangement protects against dislodged embolic material from entering the right hemisphere and left anterior circulation to the patient's brain, and also protects dislodged embolic material entering the left posterior circulation of the brain and other arteries from the heart to the patient (kidneys, liver, legs, etc.), c) the filter arrangement does not require additional blood vessel access during the deployment of the prosthetic heart valve, thus does not increase the potential for vascular access complications, d) the filter arrangement does not interfere with the TAVR valve as the TAVR valve crosses the aortic arch, e) the filter arrangement is integrated with the heart valve delivery system and is located in-line with the longitudinal axis of the catheter portion that includes the prosthetic heart valve, f) the filter arrangement has a self-expanding filter frame (e.g., filter frame is partially or fully formed of a shape memory material, frame includes a biasing arrangement to bias the frame into the open position, etc.), g) the filter arrangement can be easily constrained in a collapsed position by the delivery system, can be easily unconstrained and deployed by the delivery system prior to the heart valve being expanded in treatment area of the heart, and can be easily be collapsed and constrained by the delivery system after the heart valve has been deployed, and/or h) the filter arrangement removes captured material from the patient when the filter arrangement is removed from the patient.

### SUMMARY OF THE DISCLOSURE

The present disclosure relates generally to medical devices and medical device applications, and particularly to a vascular medical device, and more particularly to a vascular medical device that is used with a filter arrangement that will at least partially capture dislodged material in fluid passing through the filter arrangement that is at least partially caused by the deployment of the medical device at the treatment site. In one non-limiting embodiment, the medical device is a prosthetic heart valve and the filter arrangement is used to at least partially capture dislodged emboli in the blood that is passing through the filter arrangement that is at least partially caused by the expansion of the prosthetic heart valve in a treatment area of a heart. In such non-limiting embodiment, the filter arrangement is configured to inhibit or prevent dislodged emboli that at least partially results from the expansion of the prosthetic heart valve from entering one or more arteries that are located rearwardly of the expanded prosthetic heart valve. In another and/or alternative non-limiting embodiment, the filter arrangement can be configured such that it is connected to the same delivery system as the delivery system that includes the medical device, and the filter arrangement can be deployed and/or constrained by the delivery system. In another and/or alternative non-limiting embodiment, when the medical device is not a prosthetic heart valve, the medical device can be a stent, an expandable balloon, or a medical device that includes a balloon expandable or self-expanding frame.

In one non-limiting aspect of the present disclosure, the filter arrangement that is used with an expandable medical device inhibits or prevents some or all of the dislodged emboli and/or other material from a) flowing into branch arteries located rearwardly of the prosthetic heart or from continuing down the artery that the prosthetic heat valve is being deployed during the expansion of the prosthetic heart valve in the heart, or b) from continuing down a blood vessel form the location that the medical device has been expanded. In one non-limiting embodiment, the filter arrangement is configured to protect the entire aorta that is located rearwardly of the deployed prosthetic heart valve from some or all of the dislodged emboli during the expansion of the prosthetic heart valve in the heart. In another non-limiting embodiment, the filter arrangement is a component of or part of a medical device delivery system which at least a portion (e.g., 50-99.99% and all values and ranges therebetween) or all of the filter arrangement is located proximal (e.g., rearwardly) to the inflation balloon on the inflation lumen or balloon (e.g., 0-20 mm proximal to the balloon and all values and ranges therebetween). In another non-limiting embodiment, the filter arrangement is configured such that when deployed at or near the aorta, the filter system will protect the entire aorta from some or all of the dislodged emboli during the insertion of the prosthetic heart valve in the heart. As can be appreciated, for self-expanding prosthetic heart valves, an inflation lumen or balloon may not be required. As such, when the catheter is absent a balloon, the filter arrangement in accordance with the present disclosure can be attached or secured a location that is at or near (e.g., 0-50 mm and all values and ranges therebetween) the proximal end of the prosthetic heart valve (e.g., rearwardly of the prosthetic heart valve). When the delivery system includes a medical device other than a prosthetic heart valve (e.g., medical device includes a stent, a catheter balloon, an expandable balloon, and a medical device having an expandable frame), the filter arrangement can be attached or secured a location that is at or near (e.g., 0-50 mm and all values and ranges therebetween) the proximal end of the medical device (e.g., rearwardly of the medical device).

In another and/or alternative non-limiting aspect of the present disclosure, there is provided a prosthetic heart valve (e.g., prosthetic heart valve, TAVR valve, aortic, mitral valve replacement, tricuspid valve replacement, pulmonary valve replacement, etc.) that is at least partially made of a biomedical material and optionally includes a biologically compatible coating. In one non-limiting embodiment, the medical device includes an expandable frame (e.g., self-expanding frame, balloon expandable frame), more particularly the medical device is in the form of a cardiovascular implant for the treatment of structural heart disease wherein the cardiovascular implant includes an expandable frame (e.g., self-expanding frame, balloon expandable frame), still more particularly to a medical device that is in the form of a prosthetic heart valve for the treatment of structural heart disease wherein the prosthetic heart valve includes an expandable frame (e.g., self-expanding frame, balloon expandable frame), and still yet more particularly to a medical device that is in the form of a prosthetic heart valve for the treatment of structural heart disease wherein the prosthetic heart valve includes an expandable frame (e.g., self-expanding frame, balloon expandable frame) wherein the expandable frame is partially or fully formed of a rhenium and/or hafnium containing metal alloy.

In another and/or alternative non-limiting aspect of the present disclosure, there is provided a prosthetic heart valve delivery arrangement that includes a TAVR valve (e.g., self-expanding expandable valve, balloon expandable valve), a delivery system (e.g., catheter, guide shaft, etc.) and a filter arrangement that is connected to the delivery system. As can be appreciated, the delivery system can include components in addition to the catheter and guide shaft (e.g., a handle to control delivery of the TAVR valve, a sheath and ancillary equipment to facilitate delivery of the TAVR valve, etc.). When the delivery system includes an inflation lumen or balloon, such inflation lumen or balloon is generally connected to the catheter. The catheter is typically positioned coaxially to the guide shaft such that the guide shaft and the catheter can move independently of each other (e.g., move coaxially with respect to one another), but are generally connected on the proximal most end by the handle; however, this is not required. In one non-limiting specific operation of the prosthetic heart valve delivery arrangement that includes a delivery system, a TAVR valve and a filter arrangement, the TAVR valve is crimped onto the balloon of the inflation lumen or balloon of the delivery system when the TAVR valve is balloon expandable. As such, the TAVR valve when in the crimped position is positioned generally coaxial with the catheter, the guide shaft and the balloon at the location that the TAVR valve is crimped on the balloon. When the TAVR valve is self-expanding, the catheter can be absent a balloon and the TAVR valve is crimped onto a portion of the catheter.

In another and/or alternative non-limiting aspect of the present disclosure, the filter arrangement in accordance with the present disclosure that is connected to the catheter can be configured to be collapsed and constrained in the constrained-collapsed position by moving the guide shaft partially or fully over the filter arrangement. In one non-limiting embodiment, when the filter arrangement is in the constrained-collapsed position, a portion of the filter arrangement can overlie all of a portion of the crimped TAVR valve or the filter arrangement in the constrained-collapsed position can be positioned rearwardly of the crimped TAVR valve. As can be appreciated, the guide shaft can optionally be advanced to partially or fully cover the crimped TAVR valve. Such an arrangement facilitates in ensuring that as the delivery system is advanced through the patient's anatomy, the TAVR valve does not move proximally off of the catheter. In one non-limiting method of operation, the filter arrangement can be operated as follows:

After the crimped TAVR valve is positioned on the catheter and the filter arrangement is in the constrained-collapsed position on the catheter, the delivery system can be advanced through the patient's anatomy until the TAVR valve is positioned in the treatment area of the heart.

At the intended delivery site of the TAVR (e.g., aortic annulus, etc.), one more components of the delivery system can optionally be manipulated to facilitate in the desired positioning of the TAVR valve on the catheter, and/or facilitate in the desired positioning and/or orientation of the TAVR valve at the treatment area of the heart.

When deployment of the TAVR valve is desired, and the guide shaft is optionally at least partially positioned over the TAVR valve, the guide shaft is retracted by moving the guide shaft proximally relative to the inflation lumen or balloon (e.g., rearwardly from the inflation lumen or balloon), thus freeing the TAVR valve. If the guide shaft is not located least partially positioned over the TAVR valve, then this optional step is not used.

The filter arrangement is deployed form the collapsed or closed position to the open position prior to the partial or full inflation of the balloon that causes the TAVR valve to expand at the treatment area of the heart. When the filter arrangement is positioned in the open position, the filter arrangement is configured to span the region that is rearwardly of the TAVR valve such that 50-100% (and all values and ranges therebetween) of the fluid (e.g., blood, etc.) that pass the TAVR valve flows through the filter material of the filter arrangement. In one non-limiting embodiment, the guide shaft is partially or fully positioned about the filter arrangement as the TAVR valve is moved to the treatment site in the heart; however, this is not required. When the guide shaft is partially or fully positioned about the filter arrangement as the TAVR valve is moved to the treatment site in the heart, once the TAVR valve is positioned at or near the treatment site, prior to the partial or full inflation of the balloon and the partially of full expansion of the prosthetic heart valve at the treatment site in the heart, the guide shaft is retracted from the filter arrangement to allow the filter arrangement to deploy prior to the partial or full expansion of the prosthetic heart valve at the treatment site. When the TAVR valve is self-expanding, both the TAVR valve and the filter arrangement may be deployed at the same time or nearly the same time (e.g., within 0-20 seconds and all values and ranges therebetween) in the treatment area of the heart. When the TAVR valve is not a self-expanding valve, the filter arrangement is typically deployed in the open position before the partial or full expansion of the TAVR valve. As can be appreciated, the delivery system can optionally include one or more components that can be used to delay expansion of the TAVR valve until after the filter arrangement has been deployed in the open position; however, this is not required. Because the filter arrangement is connected to the delivery system, the use of the filter arrangement does not require additional vascular access and protects the patient's entire arterial system during deployment of the TAVR valve. The deployment of the filter arrangement at a location that is rearward of the TAVR valve facilitates in ensuring that some or all of the emboli that is dislodged during the expansion of the TAVR valve in the treatment area of the heart will be captured by the filter arrangement. When the TAVR valve is a balloon inflatable valve, the TAVR valve can be deployed in the treatment area of the heart by inflating the catheter balloon to a desired diameter. During the expansion of the TAVR valve, emboli can become dislodge around the treatment area and move rearwardly of the TAVR valve by the blood flow past the TAVR valve. The deployed filter arrangement located rearwardly of the TAVR valve is configured to capture such dislodged emboli that has a size that is greater than the size of material that the filter material of the filter arrangement is configured to filter.

After deployment of the TAVR valve, the balloon, when used, is deflated thereby leaving the expanded TAVR valve in place in treatment area of the heart. When the TAVR valve is a self-expanding device, inflation of the catheter balloon is not required or the catheter may be absent a balloon.

After the TAVR valve has been deployed (e.g., expanded) in the treatment area of the heart, the filter arrangement is repositioned form the open position to the constrained-collapsed or closed position on the catheter. The filter arrangement can be repositioned in the constrained-collapsed or closed position on the catheter by moving the guide shaft forwardly such that the guide shaft partially or fully covers or overlies the filter arrangement. As can be appreciated, other or additional arrangements can be used to cause the filter arrangement to be repositioned form the open position to the constrained-collapsed or closed position on the catheter. Most or all of the captured dislodge emboli by the filter arrangement is thus retained by the filter arrangement when the filter arrangement is repositioned in the constrained-collapsed or closed position on the catheter.

After the filter arrangement is partially or fully repositioned in the constrained-collapsed or closed position on the catheter, the delivery system is removed from the patient along with the dislodge emboli that remains captured by the filter arrangement.

In another and/or alternative non-limiting aspect of the present disclosure, the filter arrangement includes a filter frame that is attached (e.g., permanently or releasably detached) to the catheter, inflation lumen or balloon of the delivery system, or some other component of the delivery system. Generally, the attachment location of the filter arrangement is located at or proximal to the balloon on the inflation lumen or balloon (e.g., rearwardly of the inflation lumen or balloon) when the catheter includes a balloon (e.g., 0-50 mm rearwardly of the balloon); however, this is not required. When the catheter is absent a balloon, the attachment location of the filter arrangement is located at or proximal to the medical device on the catheter (e.g., 0-50 mm rearwardly of the medical device). In one non-limiting embodiment, the filter frame is configured that when the filter frame is not constrained (e.g., constrained by a partially or fully surrounding sheath, constrained by a partially or fully surrounding guide shaft, etc.), the filter frame will deploy into a shape that encompasses most or all of the cross-sectional area of the vascular region (e.g., 51-100% and all values and ranges therebetween of the cross-sectional area of the vascular region) located rearwardly of the deployed medical device (e.g., encompasses most or all of the cross-sectional area of the ascending aorta or other valve regions of the heart that the prosthetic heart valve is to be deployed, etc.). The filter frame can optionally be formed of a shape memory material (e.g., Nitinol, etc.) such that when the filter frame is not constrained, the filter frame expands or otherwise deploys to the near or fully expanded or open state or orientation. As can be appreciated, the filter frame can alternatively or additionally optionally employ a biasing arrangement (e.g., spring arrangement, etc.) that biases the filter frame in the near or fully expanded or open state or orientation. As can be appreciated, the filter frame can be formed of a variety of materials (e.g., Nitinol, shape memory materials, plastic, metal, composite materials, ceramic, bone, wood, etc.).

In another and/or alternative non-limiting aspect of the present disclosure, the configuration of the filter frame of the filter arrangement is non-limiting. In one non-limiting configuration, the filter frame in combination with the filter material optionally forms a generally umbrella-shaped filter arrangement or a cone-shaped filter arrangement. Such umbrella-shaped filter arrangement or a cone-shaped filter arrangement can includes two or more (e.g., 2-14 members and all values and ranges therebetween) longitudinal members or legs (e.g., Nitinol members or legs, metal alloy members or legs, plastic member or legs, composite members or legs, etc.) that are fixed or otherwise connected at or near the proximal end to the inflation lumen or balloon (e.g., rearwardly of the inflation lumen or balloon), or at or near the proximal end of the medical device on the catheter (e.g., rearwardly of the TAVR or other type of medical device). In one non-limiting arrangement, the longitudinal members or legs are fixed or otherwise connected to the catheter at a location that is rearwardly of the TAVR, balloon or other type of medical device on the catheter (e.g., 0.01-50 mm and all values and ranges therebetween rearwardly of the TAVR, balloon or other type of medical device on the catheter). The adjacently positioned longitudinal members or legs can optionally be positioned equidistant for one another around the circumference of the catheter or the inflation lumen or balloon. The longitudinal members can optionally be configured such that the longitudinal members or legs can expand or pivot outwardly from the catheter when the longitudinal members or legs are not constrained so as to form a shape or configuration (e.g., a generally umbrella-shaped filter arrangement or a generally cone-shape filter arrangement, etc.) that can be used to capture materials when the filter arrangement is deployed. As such, when a first end of the longitudinal members or legs are fixed or otherwise connected to the catheter, the second end of one or more or all of the longitudinal members or legs can move outwardly from the outer surface of the catheter (e.g., move 0.1-36 mm from the outer surface of the catheter and all values and ranges therebetween). A shape memory material or some type of mechanical arrangement (e.g., biasing arrangement [e.g., spring, etc.], gear arrangement, pulley arrangement, wire pull arrangement, magnet arrangement, etc.), hydraulic arrangement (e.g., hydraulic valve system to cause opening and/or closing of the frame, hydraulic pressure system to cause opening and/or closing of the frame, etc.), micromotor arrangement, micromotor and gear arrangement, etc. could be used to cause the longitudinal members or legs to expand or pivot outward when not partially or fully constrained. The filter material is adhered to or otherwise connected to the filter frame at one of more locations of the longitudinal members or legs. As can be appreciated, certain arrangements can be used to controllably move the longitudinal members or legs between the open and closed orientations with having to partially or fully constrained the longitudinal members or legs. For example, when the longitudinal members or legs are not biased in the open orientation, constraining of the longitudinal members or legs in the closed orientation may not be required when moving the TAVR or other medical device to a treatment site and thereafter removing the catheter from the treatment site and patient.

In another and/or alternative non-limiting aspect of the present disclosure, the filter arrangement filter arrangement optionally includes a loop filter frame arrangement. In one non-limiting configuration, the filter frame includes one or more self-expanding loops (e.g., Nitinol wire loop, shape memory material loos, etc.). Size of each loop is about 5-60 mm (and all values and ranges therebetween); however, other sizes can be used. Each of the loops is adhered to or otherwise connected at or near the proximal end of the inflation lumen, balloon and/or medical device (e.g., rearwardly of the inflation lumen, balloon and/or medical device) so that when the filter arrangement is deployed, the expanded end of the deployed filter arrangement will receive blood flow and filter such blood at a location that is rearwardly of the inflation lumen, balloon and/or medical device. The filter material is adhered to or otherwise connected to the filter frame at one of more locations of the loop frame. The proximal end of the filter arrangement can be adhered to or otherwise connected to the proximal end of inflation lumen, balloon and/or medical device; however proximal end of the filter arrangement is typically connected at or some location rearwardly of the inflation lumen, balloon and/or medical (e.g., 0.01-50 mm and all values and ranges therebetween rearwardly of the inflation lumen, balloon and/or medical device). In one non-limiting configuration of the filter arrangement, the filter frame and the filter material are folded and held in the constrained position by the guide shaft. When the guide shaft is retracted, the one or more filter frame loops open to the deployed state, thereby opening the filter arrangement to the open or deployed orientation to filter blood that flows through the deployed filter arrangement. When the filter arrangement is to be retrieved or removed from the treatment site or patient, the guide shaft can be advanced over a portion or all of the filter arrangement thereby constraining the filter frame and filter material and causing the filter arrangement to move to the constrained-collapsed or closed position.

In another and/or alternative non-limiting aspect of the present disclosure, the filter arrangement optionally includes a clamshell filter or inverse-clamshell filter configuration. In one non-limiting embodiment, the filter frame includes two self-expanding wires (e.g., Nitinol wires, shape memory material wires, etc.) that are each formed into a partial circles (e.g., half circles, three-quarter circles, etc.) at a desired deployed diameter (e.g., 10-60 mm and all values and ranges therebetween), and with legs that extend from the outermost diameter to the diameter of the inflation lumen, balloon and/or catheter. In another non-limiting embodiment, the legs of the partial circular wires can be configured to run parallel to the inflation lumen, balloon and/or catheter to facilitate in the adhering or connecting of the filter frame to the inflation lumen, balloon and/or catheter. In one non-limiting configuration, both partial circular wires are positioned such that the open ends create a full circle when deployed with the legs extending to the inflation lumen, balloon and/or catheter and the wires are attached or connected to the inflation lumen, balloon and/or catheter. The filter frame can optionally be configured such that the partial circle portions of the wires are slightly angled distally to facilitate in positioning the filter arrangement in the constrained-collapsed or closed position (e.g., making it easier for the filter frame to fold when the guide shaft is advanced over all or a portion of the filter arrangement). The filter material is generally attached or connected to the partial circle wire portions of the filter frame to create a partial or continuous connection. The proximal end of the filter arrangement can be adhered to or otherwise connected to the proximal end of inflation lumen, balloon and/or medical device; however, the proximal end of the filter arrangement is typically connected at or some location rearwardly of the inflation lumen, balloon and/or medical (e.g., 0.01-50 mm and all values and ranges therebetween rearwardly of the inflation lumen, balloon and/or medical device). When the filter frame is deployed, the shape formed by the filter frame and filter material have a funnel-type shape that has a wide opening at the distal end of the filter arrangement, and which opening reduces in size toward the location that the filter frame that is connected to the inflation lumen, or balloon or catheter. When filter arrangement is deployed, the filter frame can be configured to hold the filter material open and against the surrounding vascular wall, thereby creating a funnel that the blood flows through to filter material as the blood flows through the filter material so that the filter material captures dislodged emboli from the blood flowing through the filter material. During use, the filter frame and filter material can be folded and the guide shaft can be partially or fully advanced over the filter arrangement to hold the filter arrangement in the constrained-collapsed or closed position while delivering the medical device (e.g., prosthetic heart valve, etc.) to the target deployment site. Thereafter, the filter arrangement can be deployed at the treatment location into the open position or orientation by partially or fully retracting the guide shaft from the filter arrangement. The self-expanding properties of the self-expanding wires of the filter frame can facilitate the opening and holding the filter frame and the filter material in the open or deployed position during deployment of the filter arrangement. When the filter arrangement is to be retrieved, the guide shaft can be advanced over a portion or all of the filter arrangement thereby causing the filter frame to collapse and be fold back to the constrained-collapsed or closed position, and thereafter, the catheter can be removed from the treatment site or patient.

In another and/or alternative non-limiting aspect of the present disclosure, the filter arrangement optionally includes a filter frame that has pressurized filter frame elements. In one non-limiting embodiment, the filter frame can be optionally constructed of one or more hollow frame members, which hollow frame members can be pressurized with fluid to change the shape of the hollow pressure members. When the frame arrangement is in the collapsed-constrained or closed position, one or more of the hollow frame members can be maintained under vacuum to cause the hollow frame members to partially or fully collapse into collapsed-constrained or closed position. When one or more of the hollow frame members are pressurized, the pressured hollow frame members would be caused to expand to a second shape that is larger than the shape when not pressurized, thereby causing the pressurized hollow frame member to move to the open or deployed position. The hollow frame members can be partially or fully be formed of a plastic material (e.g., nylon, etc.) that allows the hollow frame members to have a variable stiffness based upon the pressure applied to the hollow frame members (See US 2009/0163851 for a non-limiting example of such a plastic, which is fully incorporated herein by reference). However, other or additional materials can be used to form the hollow frame members (e.g., shape memory materials, metals, composite materials, etc.). The proximal end of the filter arrangement could be adhered to or otherwise connected to the proximal end of inflation lumen, balloon and/or medical device; however proximal end of the filter arrangement is typically connected at or some location rearwardly of the inflation lumen, balloon and/or medical (e.g., 0.01-50 mm and all values and ranges therebetween rearwardly of the inflation lumen, balloon and/or medical device). In one non-limiting configuration, the expansion of the one or more hollow frame members causes the filter frame to move to the open or deployed position. In another non-limiting configuration, the removal or pressure or subjecting a vacuum to one or more of the hollow frame members causes the partial or full collapse of such hollow frame member or reduction in size of such hollow frame member, thereby causing such hollow frame members to move to the collapsed or closed position and/or enable the pressured hollow frame members to be moved the collapsed or closed position by moving the guide shaft partially or fully over the filter arrangement and/or by using another type of arrangement to move the frame members to the collapsed or closed position.

In another non-limiting embodiment, the filter material of the filter arrangement can be attached to one or more portions of the frame (e.g., the open to flow or inflow end, all or a portion of the frame members). The filter material can also be attached to the catheter, inflation lumen, balloon and/or other portion of the delivery system (e.g., on or rearwardly of the inflation lumen or balloon, rearwardly of the medical device on the catheter, etc.). The arrangement in which the filter material is connected to the filter frame is non-limiting (e.g., stitching, melted connection, adhesive connection, clamp arrangement, encapsulation of a portion or all of the frame, lamination, tongue and groove connection, solder connection, weld connection, band connection, wrapped cord connection, connection sleeve, magnet connection, hook and loop fastener, etc.). Likewise, the arrangement in which the filter material is connected to the catheter, inflation lumen, balloon and/or other portion of the delivery system is non-limiting (e.g., stitching, melted connection, adhesive connection, clamp arrangement, encapsulation of a portion or all of the catheter or balloon, lamination, tongue and groove connection, solder connection, weld connection, band connection, wrapped cord connection, connection sleeve, magnet connection, hook and loop fastener, etc.). In one non-limiting arrangement, the proximal end of the filter arrangement can be adhered to or otherwise connected to the proximal end of inflation lumen, balloon and/or medical device; however proximal end of the filter arrangement is typically connected at or some location rearwardly of the inflation lumen, balloon and/or medical (e.g., 0.01-50 mm and all values and ranges therebetween rearwardly of the inflation lumen, balloon and/or medical device).

In another and/or alternative non-limiting aspect of the present disclosure, the filter material used in the filter arrangement is typically a biocompatible material. The material used to form the filter material is non-limiting (e.g., polyurethane, nylon, polyethylene terephthalate, polystyrene, polypropylene, UHM polymers, nylon, nitinol, shape memory material, etc.). Non-limiting examples of filter material include a) polyurethane film with a thickness in the range of 0.001-0.01 inches (and all values and ranges therebetween) having a plurality of holes, (e.g., laser drilled holes or patterns, etc.), b) an expanded PTFE film (e.g., fully sintered) with a thickness in the range of 0.001-0.01 inches (and all values and ranges therebetween) having a plurality of holes, (e.g., laser drilled holes or patterns, etc.), c) PTFE film (e.g., unsintered) that is used to partially or fully encapsulate the filter frame (e.g., two layers of PTFE film partially or fully encapsulate the frame, and thereafter the two layers are connected together (e.g., sintered together, pressure connected together and heated, etc.), and then a plurality of holes, (e.g., laser drilled holes or patterns, etc.) would be formed in the PTFE film, d) a nylon material (e.g., nylon mesh, lamination of nylon mesh layers, lamination of nylon mesh, etc.), or e) a nylon material laminated to other polymer film (e.g., polyurethane, etc.), wherein the nylon material provides additional strength to the polymer film and the polymer film provides the defined porosity of the laminated material.

In another and/or alternative non-limiting aspect of the present disclosure, the filter material used in the filter arrangement has an average pore size of generally no larger than 450 microns, and is generally 50-450 microns (and all values and ranges therebetween); however, other average pore sizes can be used. In another non-limiting embodiment, the filter material is configured to prevent particles having a size that is greater than 450 microns from passing through the filter material. In one non-liming configuration, the filter material is configured such that the maximum particle size that can pass through the filter material is 50-450 microns (and all values and ranges therebetween). The thickness of the filter material is non-limiting. In one non-limiting configuration, the filter material is configured to prevent material or particles from passing through the filter material that is greater than or equal to 450 μm. In another non-limiting configuration, the filter material is configured to prevent material or particles from passing through the filter material that is greater than or equal to 300 μm. In another non-limiting configuration, the filter material is configured to prevent material or particles from passing through the filter material that is greater than or equal to 200 μm. In another non-limiting configuration, the filter material is configured to prevent material or particles from passing through the filter material that is greater than or equal to 100 μm. In another non-limiting configuration, the filter material is configured to prevent material or particles from passing through the filter material that is greater than or equal to 50 μm.

In another and/or alternative non-limiting aspect of the present disclosure, the filter material used in the filter arrangement is configured to allow at least 0.25 L/min. (0.25-50 L/min. and all values and ranges therebetween) of fluid flow (e.g., blood flow, saline fluid flow, water flow, distilled water flow, etc.) through the filter material when the filter arrangement is deployed at a treatment location (e.g., heart of a patient, blood vessel of a patient). In another non-limiting configuration, the filter material is configured to allow at least 1 L/min. of fluid flow (e.g., blood flow, saline fluid flow, water flow, distilled water flow, etc.) through the filter material. In another non-limiting configuration, the filter material is configured to allow at least 2 L/min. of fluid flow (e.g., blood flow, saline fluid flow, water flow, distilled water flow, etc.) through the filter material.

In another and/or alternative non-limiting aspect of the present disclosure, the filter material used in the filter arrangement is formed of a flexible material. The use of a flexible material a) facilitates in the unfolding/folding of the filter material when the filter arrangement moves between the open or deployed position and the closed position, b) minimizes or prevents damage to the filter material when the filter arrangement moves between the open or deployed position and the closed position, and/or c) facilitates in enabling (and does not inhibit or prevent) the filter arrangement to move between the open or deployed position and the closed position.

In another and/or alternative non-limiting aspect of the present disclosure, the filter material used in the filter arrangement has a shape that can optionally be modified to improve the interaction of the filter material with fluid flow (e.g., ensure the filter material is forced toward the blood vessel wall instead of to the center of the blood vessel or treatment site when the filter arrangement is deployed). In one non-limiting configuration of the filter material, the “lip” on the distal outermost edge of the filter material can be configured to cause the filter material to deflect outward towards the blood vessel wall or wall of the treatment site when the filter arrangement is deployed and when fluid (e.g., blood, etc.) is flowing through the filter material. In another non-limiting configuration, the filter arrangement can optionally include one or more regions that are absent filter material to allow a certain degree of fluid flow (e.g., blood flow, etc.) through the filter arrangement without being filtered by the filter material while filtering a different portion of fluid flowing through the filter arrangement by the filter material. For example, in laminar flow, the blood flow is in a blood vessel is highest at the center of the blood vessel and slowest at the blood vessel wall. In laminar flow through the blood vessel, any dislodged emboli would be drawn towards the center of the deployed filter arrangement as the blood flows through the filter arrangement. As such, if edges of the outermost area near the blood vessel wall were free of filter material when the filler arrangement is in the open or deployed position, a portion of blood would be able to freely flow around the outer edges of the deployed filter arrangement while the filter arrangement still filters some or all of the emboli in the blood that flows through the deployed filter arrangement.

In another and/or alternative non-limiting aspect of the present disclosure, the filter material used in the filter arrangement and the filter frame are optionally configured to be a) folded and constrained between the guide shaft and inflation lumen or balloon while being delivered to the target deployment site (e.g., treatment area in the heart, etc.), and/or b) folded and constrained between the guide shaft and the catheter while being delivered to the target deployment site (e.g., treatment area in the heart, etc.). In such non-limiting arrangements, the filter arrangement can be deployed at the deployment site by retracting the guide shaft from the filter arrangement that was maintaining the filter arrangement in the constrained-collapsed or closed position. The retraction of the guide sheath from the filter arrangement enables the filter frame to expand (e.g., the Nitinol frame to expand, biased frame to expand, etc.) to a deployed or open or expanded state. When the filter arrangement is to be removed from the patient, the guide shaft can optionally be advanced over a portion or all of the filter arrangement to cause the guide shaft to partially or fully cover the filter arrangement to thereby cause the filter arrangement to collapse and once again be constrained between the inner surface of the guide shaft and the outer surface of the inflation lumen, balloon and/or catheter.

In another and/or alternative non-limiting aspect of the present disclosure, the filter arrangement filter arrangement optionally includes one or more structural elements that are formed of a material that is more rigid than the filter material (e.g., metal, stiff plastic such as, but not limited to, polyimide, etc.). Such one or more optional structural elements could optionally be placed on the outside and/or inside surface of the filter material along a portion of the longitudinal length of the filter arrangement to aid in inhibiting or preventing the filter material from being snagged on the guide shaft (e.g., inhibiting or preventing the filter material from getting snagged on the leading edge of the guide shaft, etc.). The configuration of such optional one or more structural elements could be comprised of a braid, individual longitudinal elements or a spiral wound element; however, other shapes can be used. The one or more structural elements can be connected to the filter material in any number of ways (e.g., stitching, melted connection, adhesive connection, clamp arrangement, lamination, tongue and groove connection, solder connection, weld connection, band connection, wrapped cord connection, connection sleeve, magnet connection, hook and loop fastener, etc.).

In another and/or alternative non-limiting aspect of the present disclosure, the filter arrangement filter arrangement optionally includes one or more stringers that are attached near or at the proximal end of the inflation lumen, balloon and/or catheter (e.g., located rearwardly of the inflation lumen or balloon) and/or at a location that is distal to the distal end of the filter arrangement (e.g., forwardly of the filter arrangement) and which stringers extend to reach the open end of the filter arrangement. The purpose of the stringers is to reinforce the filter frame to hold it open against blood flow when the filter arrangement is deployed. The one or more stringers can be made of metal (e.g., Nitinol, stainless steel, etc.), plastic (polyimide, nylon, etc.), or suture type material. The one or more stringers can be connected to the filter frame and/or near or at the proximal end of the inflation lumen, balloon and/or catheter in any number of ways (e.g., stitching, melted connection, adhesive connection, clamp arrangement, lamination, tongue and groove connection, solder connection, weld connection, band connection, wrapped cord connection, connection sleeve, magnet connection, hook and loop fastener, etc.).

In another and/or alternative non-limiting aspect of the present disclosure, there is provided a non-limiting method for the use of the prosthetic heart valve delivery arrangement that includes a prosthetic heart valve, a delivery system, and a filter arrangement. In one non-limiting method, the method includes:

Providing a filter arrangement that is attached or otherwise connected to a catheter, and wherein the catheter includes a guide shaft.

The filter frame and filter material of the filter arrangement are folded and the guide shaft on the catheter is advanced partially or fully over the filter arrangement so as to maintain the filter arrangement in a constrained-collapsed or closed position.

The delivery system with the medical device on the catheter of the delivery system (e.g., crimped prosthetic heart valve on the balloon of the inflation lumen or balloon, etc.), is advanced through the patient's anatomy while the filter arrangement and optionally the medical device are in constrained or closed o unexpanded or crimped position until the medical device is positioned in the desired delivery location (e.g., treatment area in the heart, etc.).

Once the medical device has been positioned at or near the treatment site (e.g., treatment site or area in the heart, etc.), the filter arrangement is deployed in the treatment site or area at a location that is proximal to the medical device (e.g., rearwardly of the prosthetic heart valve). The filter arrangement can be deployed by the retraction of the guide shaft from the filter arrangement, thereby allowing the filter arrangement to expand from the constrained-collapsed or closed position to the expanded or open or deployed state at or near the treatment site or area. In the expanded or open or deployed state, the filter frame causes the filter material to cover most or all of the cross-sectional area (80-100% of the cross-sectional area and all values and ranges therebetween) of a location in the treatment site or area (e.g., ascending aorta, region of heart, etc.) that is proximal to the medical device (e.g., a location rearwardly of the medical device) where the filter arrangement is expanded. Such coverage of the filter material across the cross-sectional area of the location that is proximal to the medical device (e.g., a location rearwardly of the medical device) results in all of nearly all (e.g., 70-100% and all values and ranges therebetween) of the blood flowing past the medical device after the filter arrangement has been deployed in the treatment site or area to flow through the filter material of the filter arrangement. When the filter arrangement is in the deployed or open position, the filter material forms a barrier to certain sized particles in the blood as the blood passes through the filter material. As such, as blood flows through the deployed filter arrangement, particles or materials in the blood that are larger than the pore size of the filter material or larger than the size of particles that the filter material is allowed to pass through the filter material are captured by the filter material.

Deploy the medical device at the treatment site (e.g., expand the prosthetic heart valve at the treatment site, etc.). When the medical device is a balloon expandable medical device, the medical device is deployed from a crimped orientation to an expanded orientation by the inflation of the balloon on the catheter. If the medical device is a self-expanding device, a constricting component (e.g., guide shaft, sheath, etc.) is retracted or otherwise removed from the medical device to enable the medical device to be deployed from the crimped orientation to the expanded orientation. If the medical device is a self-expanding device, the medical device may be partially or fully expanded as the same time as the filter arrangement; however, this is not required.

After the medical device is deployed at the treatment site, the filter arrangement is collapsed and constrained to the constrained-collapsed or closed position so that the filter arrangement can be removed from the treatment site or area. In one non-limiting embodiment, the filter arrangement can be collapsed and constrained to the constrained-collapsed or closed position by advancing the guide shaft over a portion or all of the filter arrangement. During the partial or full constrainment of the filter arrangement, the filter frame is caused to partially or fully fold or collapse onto the inflation lumen, balloon and/or catheter. Most or all of the embolic material that was captured by the filter material when the filter arrangement was deployed is retained in the filter arrangement as the filter arrangement moves from the deployed position to the constrained-collapsed or closed position. When the filter arrangement moves from the deployed or open position to the constrained-collapsed or closed position, the particles or material captured by the filter material are partially or fully enclosed by and/or within the filter material and remain trapped in the filter material (e.g., trapped between the filter material and the inflation lumen or balloon, etc.) as the delivery system is removed from the patient.

Remove the catheter and delivery arrangement while the delivery arrangement is in the constrained-collapsed or closed position from the treatment site or area and/or from the patient.

In another and/or alternative non-limiting aspect of the present disclosure, a non-limiting method for using the medical device delivery arrangement that includes a prosthetic heart valve, a delivery system, and a filter arrangement includes the steps of:

Use the delivery device to deliver the prosthetic heart valve to a desired location in the heart while the filter arrangement is in the constrained-collapsed or closed position and the prosthetic heart valve is in a crimped position.

Optionally orient the prosthetic heart valve to obtain the desire commissure alignment at the treatment site.

Begin pacing (cardiac output→□0) Retract guide shaft to deploy filter arrangement to the deployed or open position.

Confirm deployment of the filter arrangement.

Inflate the balloon of the catheter to deploy and expand the prosthetic heart valve in the heart at the treatment site.

Confirm desired deployment of the prosthetic heart valve at the treatment site.

Deflate balloon on the catheter.

Confirm the prosthetic heart valve placement in the heart.

Stop pacing.

Advance guide shaft to collapse and capture the filter arrangement in the constrained-collapsed or closed position.

Retract the entire delivery system and remove from the patient while the expanded prosthetic heart valve remains deployed at the treatment site.

In another and/or alternative non-limiting aspect of the present disclosure, the prosthetic heart valve delivery arrangement in accordance with the present disclosure that includes a prosthetic heart valve, a delivery system, and a filter arrangement has one or more of the following non-limiting advantages over prior art prosthetic heart valve delivery arrangements:
a. The filter arrangement is integrated with the delivery system for the medical device (e.g., prosthetic heart valve, etc.), thus separate procedures are not required to use the filter arrangement with the medical device during deployment of the medical device at the treatment site. For example, the Sentinel™ device offered by Bostin Scientific is a device that is separately deployed from the TAVR valve, thus requiring a first delivery device to deploy the Sentinel™ device in the right subclavian vessel and in the left carotid artery, and a second delivery device that includes the TAVR for insertion into the patient and the deployment of the TAVR in the heart. As such, the need to use multiple delivery devices and the added time required for use of the multiple delivery device increases the risk of injury to a patient during the plurality of procedures. The integration of the filter arrangement with the medical device delivery arrangement in accordance with the present disclosure eliminates the use of multiple deployment devices, thus reduces the time for deployment and the removal of the delivery system and filter arrangement from the treatment site and patient after the medical device is deployed in the treatment area. The integration of the filter arrangement with the medical device delivery arrangement in accordance with the present disclosure eliminates the use of multiple deployment devices, thus also reduces the risk of injury to the treatment area of the patient due to multiple delivery devices being used to insert a filter and a medical device to the treatment area. Furthermore, the elimination of the need to use multiple deployment devices eliminates the need for additional vascular access and procedural steps to deploy the filter arrangement and the medical device at the treatment area. The integration of the filter arrangement with the medical device delivery arrangement in accordance with the present disclosure reduces extended aortic arch dwell times of the filter arrangement in the treatment area of the heart, which extended aortic arch dwell times increase the risk of thrombosis and iatrogenic emboli that can result from the scraping of aortic arch or carotids. The integration of the filter arrangement with the medical device delivery arrangement in accordance with the present disclosure eliminates the need for additional vessel access (e.g., radial access) for the delivery of the filter arrangement and medical device to the treatment site, which additional vessel access increases the potential for vascular access complications, and can interfere with the proper deployment of the medical device (e.g., interfere with TAVR valve insertion and deployment as the TAVR valves crosses the aortic arch due to the separate filters and filter deliver devices that are already positioned in the blood vessel and heart as the TAVR valve is moved to the heart and then deployed in the heart). The integration of the filter arrangement with the medical device delivery arrangement in accordance with the present disclosure also allows for easier and more effective deployment of the filter arrangement in the treatment area. The filter arrangement in accordance with the present disclosure is configured to be positioned about the longitudinal axis of the catheter and is located at or near the medical device that is to be deployed at the treatment area. The filter arrangement is typically connected to the catheter, thus does not require a separate delivery system to the treatment area or site. A sheath or guide shaft can be removably positioned about a portion or all of the filter arrangement to facilitate in a) the constriction of the filter arrangement in the constrained-collapsed or closed position during the delivery of the filter arrangement to the treatment area or site, b) deployment of the filter arrangement at the treatment area from the constrained-collapsed or closed position to the deployed or open position, and/or c) the collapsing and constriction of the filter arrangement from the deployed or open position to the constrained-collapsed or closed position during the removal of the filter arrangement from the treatment area or site. The filter arrangement in the constrained-collapsed or closed position can be easily guided by the catheter along with the medical device to the treatment area and then deployed in-line with the medical device, thus ensuring the proper deployment location of the filter arrangement (e.g., rearwardly of the medical device), and then easy collapsing and constraining the filter arrangement for removal of the filter arrangement from the patient with the catheter after deployment of the medical device at the treatment area. The deployed filter arrangement can be configured to deploy about the central longitudinal axis of the catheter at or near the location of deployment of the medical device.
b. The filter arrangement in accordance with the present disclosure inhibits or prevents most (e.g., 80-100% and all values and ranges therebetween), if not all, of the emboli and/or other material that becomes dislodged during the deployment of the medical device at a treatment site or area, that is greater than the size material that the filter material is configured to allow to pass through the filter material, from passing through the filter arrangement and into the vascular system that is located rearwardly of the filter arrangement. As such, when the medical device is a prosthetic heart valve that is deployed in the heart, and during the deployment of the prosthetic heart valve, emboli material can be dislodged from the heart. When the deployed filter arrangement is positioned rearwardly of the deployed prosthetic heart valve and forwardly of other arteries located rearwardly of the deployed prosthetic heart valve, the filter material captures most, if not all, of the dislodge emboli material that is greater or larger in size than what the filter material is configured to allow to pass through the filter material. For instance, if the filter material is configured to prevent particles that are greater than 100 microns from passing through the filter material, then dislodged emboli material that is larger than 100 microns will be captured by the deployed filter arrangement that is located rearwardly of the prosthetic heart valve as the blood that includes such sized or larger material flows through the filter material of the filter arrangement. Such a filter arrangement that is integrated with the medical device delivery arrangement is a significant advantage over prior art filters that only capture dislodge emboli flowing into select arteries after a TAVR valve has been expanded at a treatment area. For example, the Sentinel™ device is only deployed in the right subclavian vessel and in the left carotid artery. These two arteries feed blood to the brain. However, other arteries from the heart feed blood to other regions of the body. As such, the Sentinel™ device does not prevent dislodged emboli from passing into arteries other than the right subclavian vessel and the left carotid artery when the Sentinel™ device is deployed during a TAVR procedure. The dislodged emboli that pass into these other arteries can cause blood clots, interfere with proper blood flow, and/or damage or otherwise injury organs. The filter arrangement in accordance with the present disclosure prevents most, if not all, of the dislodged emboli from passing into any of the arteries located rearwardly to the deployed prosthetic heart valve, which dislodged emboli is greater in size than what the filter material is configured to allow to pass through the filter material.
c. The filter arrangement in accordance with the present disclosure is configured to capture most (e.g., 80-100% and all values and ranges therebetween), if not all, of the emboli that becomes dislodged during the deployment of the medical device that is greater size than what the filter material is configured to allow to pass through the filter material. For example, and as discussed above, the Sentinel™ device is only deployed in the right subclavian vessel and in the left carotid artery. However, other arteries feed blood to other regions of the body. As such, the Sentinel™ device does not prevent dislodged emboli from passing into arteries other than the right subclavian vessel and the left carotid artery when the Sentinel™ device is deployed during a TAVR procedure. The dislodged emboli that pass into these other arteries can cause blood clots, interfere with proper blood flow, and/or damage or otherwise injury organs. The shape of the deployed filter arrangement and the positioning of the filter material at or near the treatment area or site is configured to facilitate in the capture of most or all of the dislodged emboli from the deployment of the medical device at the treatment site or area that is greater size than what the filter material is configured to allow to pass through the filter material. The shape of the deployed filter arrangement can be conical shaped, funnel-shaped, cone shape, conoid, and the like; however, other shapes can be used. Such shapes are used to capture and retain the dislodged emboli as the blood flows through the filter material. Some prior art filter arrangements are configured to merely deflect dislodged materials to prevent or inhibit such material from flowing into a certain blood vessel. However, such deflected material is allowed to flow into other blood vessels, which material can cause interference with blood flow or damage or injury to the blood vessel and/or organs in the body. The capture and retention of the material by the filter arrangement in accordance with the present disclosure is a significant enhancement and advantage over a) prior art filters that are configured to partially or fully deflect, but not capture dislodged material, and b) prior art filters that are configured to only filter dislodged material in only one or two, but not all, of the arteries of the heart located rearwardly to the TAVR valve being deployed.
d. The filter arrangement in accordance with the present disclosure is configured to remove most (e.g., 80-100% and all values and ranges therebetween), if not all, of the captured emboli from the patient after the prosthetic heart valve is deployed. As discussed above, some prior art filters are configured to only deflect some or all of the dislodge materials during a prosthetic heart valve procedure. Such deflected materials can cause problems and/or injury in other regions of the body. Also, some filters are configured such that during removal of the filter from the patient, previously captured material is allowed to escape from the filter and flow into the body of the patient. The configuration of the filter arrangement in accordance with the present disclosure is configured to both capture dislodged materials flowing into the filter arrangement that are larger in size than what the filter material is configured to allow to pass through the filter material, and to retain such captured particles in the filter arrangement during the removal of the filter arrangement from a patient. In one non-limiting configuration, when the filter arrangement moves from the deployed or open position to the constrained-collapsed or closed position, less than 30% (e.g., 0-30% and all values and ranges therebetween) of the captured particles by the filter material escape from the filter arrangement while in the constrained-collapsed or closed position and as the filter arrangement is removed from the patient.
e. The medical device delivery arrangement that includes a prosthetic heart valve, a delivery system, and a filter arrangement can be used to deploy a prosthetic heart valve at locations of the heart other than the aortic valve. Because the filter arrangement is integrated with the catheter that is used to deploy the prosthetic heart valve, the filter arrangement can be easily and conveniently positioned and deployed at a location that is rearward to the deployed prosthetic heart valve at any location in the heart. Some filters are only configured for use in certain locations in the heart. For example, the Sentinel™ device is designed for use during a TAVR procedure. The Sentinel™ device is not used for prosthetic heart valve procedures other than aortic valve procedures.
f. The filter arrangement in accordance with the present disclosure can be integrated in medical device delivery systems other than a prosthetic heart valve system (e.g., a delivery system that includes a stent instead of a prosthetic heart valve, a delivery system for a balloon angioplasty procedure or a balloon expansion procedure, a delivery system for a medical device that includes an expandable frame, etc.). The filter arrangement in accordance with the present disclosed is not limited to prosthetic heart valve applications, but can be used in other types of vascular procedure. The prior art heart filters are only designed for use in prosthetic heart valve applications.
g. The filter arrangement in accordance with the present disclosure can be used with a) a catheter that includes or does not include an expandable balloon, and b) various types of medical devices such as, but not limited to a stent, prosthetic heart valve, other type of expandable medical device, and/or an expandable balloon. As can be appreciated, when the filter arrangement is used with a self-expanding device (e.g., self-expanding prosthetic heart valve, self-expanding stent, etc.), a balloon on the catheter may not be required, and the filter arrangement is attached or connected at or rearward of the self-expanding device. Although the filter arrangement in accordance with the present disclosure is particularly configured for use in cardiovascular procedures, it will be appreciated that the filter arrangement can be used in other vascular procedures to filter out material that becomes dislodge during a procedure. For example, balloon angioplasty procedures in blood vessels, or balloon expansion in other vascular passageways (e.g., urethra, bladder, etc.) can result in dislodgement of materials during balloon expansion. The filter arrangement in accordance with the present invention can be used with such balloon expansion procedures to capture and remove and dislodged materials during such procedure.
h. The filter arrangement in accordance with the present disclosure can be attached or connected to the balloon or be positioned near the balloon such that when the filter arrangement is deployed and the balloon is expanded, a portion of the expanded balloon interacts with the filter arrangement to facilitate in maintaining the filter arrangement in the open and deployed position; however, this is not required.
i. The filter arrangement in accordance with the present disclosure when deployed at or near the treatment site or area has a sufficient diameter or cross-sectional area to cover 80-100% (and all values and ranges therebetween) of the cross-sectional area of the vascular passageway that is rearward (e.g., within 0-50 mm and all values and ranges therebetween) of the medical device being deployed at the treatment area. For example, the deployed filter arrangement has a sufficient diameter (e.g., deployed filter arrangement has a maximum diameter of 20-45 mm and all values and ranges therebetween) to cover the vascular passageway that is located rearward to the prosthetic heart valve that is deployed in the heart or at a location that is rearward (e.g., within 0-20 mm and all values and ranges therebetween) to the balloon on the inflation lumen or balloon.
j. The filter arrangement in accordance with the present disclosure is deployed rearward of the aortic annulus and distal or forwardly of the innominate artery or brachiocephalic artery (e.g., the deployed filter arrangement is located in front of or before the innominate artery or brachiocephalic artery) so that the filter arrangement does not block blood flow into the innominate artery or brachiocephalic artery when the medical device is a TAVR valve that is being inserted between the aortic annulus and the innominate artery or brachiocephalic artery.
k. The filter material of the filter arrangement can be configured to capture emboli material that has a size of ≥50 μm, or ≥100 μm; however, other sizes can be used.
l. The deployment of the filter arrangement at or near the treatment site or area does not cause significant movement of the position of the prosthetic heart valve in the heart.
m. After deployment of the filter arrangement, the filter arrangement does not significantly move at or near the treatment area, thus minimizing damage to surrounding tissue and/or vessels during the deployment of the filter arrangement.
n. The filter material of the filter arrangement allows for sufficient blood flow through the filter material (≥0.25 L/min., ≥2 L/min., etc.).
o. The filter arrangement is robust enough to withstand its intended use (e.g., the filter arrangement can properly be deployed and function after at least 2-3 deployments/retrievals).
p. The filter arrangement when deployed is configured to capture at least 90% (e.g., 90-100% and all values and ranges therebetween) of the dislodged embolic material having a size between 100 μm and 1 mm; however, other sizes can be used.
q. The use of the filter arrangement is not thrombogenic (usage time ≤5 min).
r. The filter arrangement provides an arrangement to confirm deployment at the treatment area and subsequent collapse and constrainment prior to removal from the treatment area (e.g., one or more radiopaque markers are located on or about the filter arrangement).
s. The filter arrangement is configured to be prepped for removal of air from the filter arrangement prior to use of the filter arrangement in a patient.
t. The filter arrangement is capable of being prepared for use by clinical field service personnel (easy to load, prepare, etc.).
u. The filter arrangement does not interfere with the function of the delivery system (e.g., not impair commissural alignment, movement of guide shaft, etc.).
v. The filter arrangement, when deployed in the aortic region of the heart, is configured to protect all four major cerebral vessels from dislodged embolic material during the deployment of the prosthetic heart valve.
w. The filter arrangement is easy to deploy and retrieve.
x. The filter arrangement is integrated with the medical device delivery system.
y. The filter arrangement maintains a stable position during deployment.

In accordance with one non-limiting aspect of the present disclosure, the prosthetic heart valve is not limited to a TAVR valve, but can be mitral valve replacement, tricuspid valve replacement, pulmonary or valve replacement. It will be appreciated that throughout this disclosure, the medical device frequently referred to is TAVR valve or prosthetic heart valve. However, it will be appreciated that other expandable medical devices can be substituted for the TAVR valve or prosthetic heart valve when the medical device is to be used in regions of the patient other than the heart. Such other medical devices that are part of this disclosure include an expandable stent, an expandable balloon, and other types of expandable devices that are expanded in a vascular passageway, and which these other medical devices are capable to be integrated on the same delivery device as the filer arrangement for delivery to a treatment area.

In accordance with one non-limiting aspect of the present disclosure, the prosthetic heart valve includes a radially collapsible and expandable frame and a leaflet structure that comprises a plurality of leaflets. In another non-limiting embodiment, the prosthetic heart valve optionally includes an annular or outer skirt that is disposed on and partially or fully covering or overlaid over the cells of at least a portion of the frame. In another non-limiting embodiment, the prosthetic heart valve optionally includes an inner skirt that is disposed on and partially or fully covering or overlaid over the cells of at least a portion of the frame. In another non-limiting embodiment, the frame of the prosthetic heart valve can comprise a plurality of interconnected axial longitudinal member, struts or angular articulating members, and strut joints that define a plurality of cells in the frame. In one non-limiting configuration, two or more or all of the cells have an open cell configuration (e.g., an open cell is defined as cell wherein at least one of the cell walls is formed of an axial longitudinal member). In another non-limiting embodiment, the frame can be partially (e.g., 1-99.999 wt. % and all values and ranges therebetween) or fully made of a metal material.

In accordance with another and/or alternative non-limiting aspect of the present disclosure, there is provided a prosthetic heart valve that includes a frame, a leaflet structure supported by the frame, an optional outer skirt secured to the frame and/or optional inner skirt, and an optional inner skirt secured to the surface of the frame, the optional outer skirt and/or leaflet structure. The prosthetic heart valve can be implanted in the annulus of the native aortic valve; however, the prosthetic heart valve also can be configured to be implanted in other valves of the heart (e.g., tricuspid valve, pulmonary valve, mitral valve). The prosthetic heart valve has a “lower” end and an “upper” end, wherein the lower end of the prosthetic heart valve is the inflow end and the upper end of the prosthetic heart valve is the outflow end. The frame of the prosthetic heart valve is configured to be radially collapsible to a collapsed or crimped state for introduction into the body (e.g., on a delivery catheter, etc.) and radially expandable to an expanded state for implanting the prosthetic heart valve at a desired location in the body (e.g., the aortic valve, tricuspid valve, pulmonary valve, mitral valve, blood vessel, etc.). The frame of the prosthetic heart valve can be formed of a plastically-expandable material that permits crimping of the frame to a smaller profile for delivery and expansion of the frame at the treatment site. The expansion of the crimped frame of the prosthetic heart valve can be by an expansion device such as, but not limited to, a balloon of on an inflation lumen or balloon; however, the frame can optionally be partially (e.g., 1-99.999 wt. % and all values and ranges therebetween) or fully formed of a self-expanding material (e.g., Nitinol, shape memory material, etc.). The frame can be at least partially (e.g., 1-99.999 wt. % and all values and ranges therebetween) formed of a plurality of struts or angular articulating members or struts, and axial longitudinal members or vertically extending longitudinal members or posts. The axial longitudinal members or vertically extending longitudinal members or posts and/or struts or angular articulating members can optionally be interconnected via a lower row of circumferentially extending struts or angular articulating members and an upper row of circumferentially extending struts or angular articulating members via strut joints. The struts or angular articulating members can be arrangement in a variety of patterns (e.g., zig-zag pattern, saw-tooth pattern, triangular pattern, polygonal pattern, oval pattern, etc.). One or more of the longitudinal posts and/or struts or angular articulating members can have the same or different thicknesses and/or cross-sectional shape and/or cross-sectional area. One or more cells of the frame can optionally have an open cell configuration (e.g., an open cell is defined as cell wherein at least one of the cell walls is formed of an axial longitudinal member or vertically extending longitudinal member or post.

In accordance with another and/or alternative non-limiting aspect of the present disclosure, there is provided a prosthetic heart valve that optionally includes an inner skirt that can be formed of a variety of flexible materials (e.g., polymer [e.g., polyethylene terephthalate (PET), polyester, nylon, Kevlar®, silicon, etc.], composite material, metal, fabric material, etc.). In one non-limiting embodiment, the material used to partially (e.g., 1-99.999 wt. % and all values and ranges therebetween) or fully form the inner skirt can optionally be substantially non-elastic (i.e., substantially non-stretchable and non-compressible). In another non-limiting embodiment, the material used to partially or fully form the inner skirt can optionally be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt can optionally be formed from a combination of a cloth or fabric material that is coated with a flexible material or with a stretchable and/or compressible material so as to provide additional structural integrity to the inner skirt. The size, configuration, and thickness of the inner skirt is non-limiting (e.g., thickness of 0.1-20 mils and all values and ranges therebetween). The outer skirt can be secured to the inside of the frame, a portion of the outside of the frame, one or more leaflets, and/or an optional outer skirt using various means (e.g., sutures, clamp arrangement, adhesive, melted connection, etc.).

In accordance with another and/or alternative non-limiting aspect of the present disclosure, there is provided a prosthetic heart valve that optionally includes an inner skirt that can be used to 1) at least partially seal and/or prevent perivalvular leakage, 2) at least partially facilitate in securing the leaflet structure to the frame, 3) at least partially protect the leaflets from damage during the crimping and/or expansion process, and/or 4) at least partially protect the leaflets from damage during the operation of the prosthetic heart valve in the heart.

In accordance with another and/or alternative non-limiting aspect of the present disclosure, there is provided a prosthetic heart valve that optionally includes an outer skirt that is positioned at least partially (e.g., 1-99.999 wt. % and all values and ranges therebetween) about the exterior region of the frame. The outer skirt generally is positioned completely around a portion of the outside of the frame. Generally, the outer skirt is positioned about the lower portion of the frame, but does not fully cover the upper half of the frame; however, this is not required. The outer skirt can be connected to the frame, and/or the optional inner skirt by a variety of arrangements (e.g., sutures, adhesive, melted connection, clamping arrangement, etc.). At least a portion of the outer skirt can optionally be located on the interior surface of the frame. Generally, the outer skirt is formed of a more flexible and/or compressible material than the inner skirt; however, this is not required. The outer skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with the stretchable and/or compressible material to provide additional structural integrity to the outer skirt. The size, configuration, and thickness of the outer skirt is non-limiting. The thickness of the outer skirt is generally at least 0.1 mils (e.g., 0.1-20 mils and all values and ranges therebetween).

In accordance with another and/or alternative non-limiting aspect of the present disclosure, there is provided a prosthetic heart valve that optionally includes an outer skirt that can be used to 1) at least partially seal and/or prevent perivalvular leakage, 2) at least partially protect the leaflets from damage during the crimping and/or expansion process, and/or 3) at least partially protect the leaflets from damage during the operation of the prosthetic heart valve in the heart.

In accordance with another and/or alternative non-limiting aspect of the present disclosure, there is provided a prosthetic heart valve that includes a leaflet structure that can be attached to the frame, the inner skirt and/or the outer skirt. The connection arrangement used to secure the leaflet structures to the frame, the inner skirt and/or the outer skirt is non-limiting (e.g., sutures, staples, melted bold, adhesive, clamp arrangement, etc.). The material used to form the leaflet structures include polymers, bovine pericardial tissue, bovine tissue, porcine tissue, biocompatible synthetic materials, or various other suitable natural or synthetic materials.

In accordance with another and/or alternative non-limiting aspect of the present disclosure, there is provided a prosthetic heart valve that includes a leaflet structure comprised of two or more leaflets (e.g., 2, 3, 4, 5, 6, etc.). In one non-limiting arrangement, the leaflet structure includes three leaflets arranged in a tricuspid arrangement. The configuration of the leaflet structures is non-limiting.

In accordance with another and/or alternative non-limiting aspect of the present disclosure, there is provided a prosthetic heart valve that includes a leaflet structure wherein the leaflets of the leaflet structure can optionally be secured to one another at their adjacent sides to form commissures of the leaflet structure (the edges where the leaflets come together). The leaflet structure can be secured together by a variety of connection arrangement (e.g., sutures, adhesive, melted bond, clamping arrangement, etc.).

In accordance with another and/or alternative non-limiting aspect of the present disclosure, there is provided a prosthetic heart valve that includes a leaflet structure wherein one or more of the leaflets can optionally include reinforcing structures or strips to 1) facilitate in securing the leaflets together, 2) facilitate in securing the leaflets to the inner skirt, the out skirt and/or frame, and/or 3) inhibit or prevent tearing or other types of damage to the leaflets.

In accordance with another and/or alternative non-limiting aspect of the present disclosure, there is provided a method for crimping a medical device having a frame. The method includes placing the medical device in the crimping aperture of a crimping device such that the frame of the medical device is disposed adjacent to the crimping jaws of the crimping device. Pressure is applied against the frame with the crimping jaws to radially crimp the medical device to a smaller profile.

In accordance with another and/or alternative non-limiting aspect of the present disclosure, the frame of the medical device is partially (e.g. 1-99.999 wt. % and all values and ranges therebetween) or fully formed of a metal material that includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, or m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, hafnium alloy, refractory metal alloy, or metal alloy that includes at least 5 atomic weight percent (awt. %) or atomic percent (awt. %) rhenium and/or hafnium (e.g., 5-99 awt. % rhenium and/or hafnium and all values and ranges therebetween). As used herein, atomic weight percent (awt. %) or atomic percentage (awt %) or atomic percent (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. It has been found that for several metal alloys the inclusion of at least 5-15 awt. % rhenium and/or hafnium results in the ductility and/or tensile strength of the metal alloy to improve as compared to a metal alloy is that absent rhenium and/or hafnium. Such improvement in ductility and/or tensile strength due to the inclusion of at least 5-15 awt. % rhenium and/or hafnium in the metal alloy is referred to as the “rhenium and/or hafnium effect.” As defined herein, a “rhenium and/or hafnium effect” is a) an increase of at least 10% in ductility of the metal alloy caused by the addition of rhenium and/or hafnium to the metal alloy, and/or b) an increase of at least 10% in tensile strength of the metal alloy caused by the addition of rhenium and/or hafnium to the metal alloy. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc. In one non-limiting arrangement, 50-100 wt. % (and all values and ranges therebetween) of the expandable frame of the medical device is formed of a refractory metal alloy or a metal alloy that includes at least 15 awt. % rhenium and/or hafnium. In another non-limiting arrangement, the metal alloy that is used to partially or fully form the expandable frame of the medical device includes at least 30 wt. % (e.g., 30-99 wt. % and all values and ranges therebetween) of one or more of molybdenum, rhenium, niobium, tantalum or tungsten. In another non-limiting embodiment, the refractory metal alloy or the metal alloy that includes at least 5-15 awt. % rhenium and/or hafnium can be used to 1) increase the radiopacity of the frame of the medical device, 2) increase the radial strength of the frame of the medical device, 3) increase the yield strength and/or ultimate tensile strength of the frame of the medical device, 4) improve the stress-strain properties of the frame of the medical device, 5) improve the crimping and/or expansion properties of the frame of the medical device, 6) improve the bendability and/or flexibility of the frame of the medical device, 7) improve the strength and/or durability of the frame of the medical device, 8) increase the hardness of the frame of the medical device, 9) improve the biostability and/or biocompatibility properties of the frame of the medical device, 10) increase fatigue resistance of the frame of the medical device, 11) resist cracking in the frame of the medical device, 12) resist propagation of cracks in the frame of the medical device, 13) enable smaller, thinner, and/or lighter weight frames of the medical device to be made, 14) facilitate in the reduction of the outer diameter of a crimped medical device, 15) improve the conformity of the frame of the medical device to the shape of the treatment area when the medical device is expanded in the treatment area, 16) reduce the amount of recoil of the frame of the medical device after the frame is expanded in the treatment area, 17) reduce adverse tissue reactions with the frame of the medical device, 18) reduce metal ion release from the frame after implantation of the medical device, 19) reduce corrosion of the frame of the medical device after implantation of the medical device, 20) reduce allergic reaction with the frame of the medical device after implantation of the medical device (e.g., reduce nickel content of metal alloy, etc.), 21) improve hydrophilicity of the frame of the medical device, 22) reduce magnetic susceptibility of the frame of the medical device, 23) reduced longitudinal foreshortening the frame of the medical device when the frame of the medical device is expanded, and/or 24) reduce toxicity of the frame of the medical device after implantation of the medical device.

In another and/or alternative non-limiting aspect of the disclosure, the frame of the frame of the medical device is optionally partially or fully formed of stainless steel, CoCr alloys, TiAlV alloys, aluminum alloys, nickel alloys, titanium alloys, tungsten alloys, molybdenum alloys, copper alloys, MP35N alloys, beryllium-copper alloys that have been modified to include at least 5-15 awt. % rhenium and/or hafnium so as to result in improved ductility and/or tensile strength as compared to the same metal alloy that is absent rhenium and/or hafnium. As defined herein, a stainless-steel alloy (SS alloy) includes at least 50 wt. % (weight percent) iron, 10-28 wt. % chromium, 0-35 wt. % nickel, and optionally one or more of 0-4 wt. % molybdenum, 0-2 wt. % manganese, 0-0.75 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, and 0-2 wt. % tungsten. A 316L alloy that falls within a stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a cobalt-chromium alloy (CoCr alloy) includes 30-68 wt. % cobalt, 15-32 wt. % chromium, and optionally one or more of 1-38 wt. % nickel, 2-18 wt. % molybdenum, 0-18 wt. % iron, 0-1 wt. % titanium, 0-0.15 wt. % manganese, 0-0.15 wt. % silver, 0-0.25 wt. % carbon, 0-16 wt. % tungsten, 0-2 wt. % silicon, 0-2 wt. % aluminum, 0-1 wt. % iron, 0-0.1 wt. % boron, 0-0.15 wt. % silver, and 0-2 wt. % titanium. As a MP35N alloy that falls within a CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a Phynox and Elgiloy alloy that falls within a CoCr alloy includes 38-42 wt. % cobalt, 18-22 wt. % chromium, 14-18 wt. % iron, 13-17 wt. % nickel, 6-8 wt. % molybdenum. As defined herein, a L605 alloy that falls within a CoCr alloy includes 18-22 wt. % chromium, 14-16wt. % tungsten, 9-11 wt. % nickel, balance cobalt. As defined herein, a titanium-aluminum-vanadium alloy (TiAlV alloy) includes 4-8 wt. % aluminum, 3-6 wt. % vanadium, 80-93 wt. % titanium, and optionally one or more of 0-0.4 wt. % iron, 0-0.2 wt. % carbon, 0-0.5 wt. % yttrium. A Ti-6A1-4V alloy that falls with a TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, an aluminum alloy includes 80-99 wt. % aluminum, and optionally one or more 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1wt. % nickel, 0-0.2 wt. % vanadium, 0-0.1 wt. % gallium, and 0-7 wt. % copper. As defined herein, a nickel alloy includes 30-98 wt. % nickel, and optionally one or more 5-25 wt. % chromium, 0-65wt. % iron, 0-30 wt. % molybdenum, 0-32 wt. % copper, 0-32 wt. % cobalt, 2-2 wt. % aluminum, 0-6wt. % tantalum, 0-15 wt. % tungsten, 0-5 wt. % titanium, 0-6 wt. % niobium, 0-3 wt. % silicon. As defined herein, a titanium alloy includes 80-99 wt. % titanium, and optionally one of more of 0-6wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a tungsten alloy includes 85-98 wt. % tungsten, and optionally one or more of 0-8 wt. % nickel, 0-5 wt. % copper, 0-5 wt. % molybdenum, 0-4 wt. % iron. As defined herein, a molybdenum alloy includes 90-99.5 wt. % molybdenum, and optionally one or more of 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a copper alloy includes 55-95 wt. % copper, and optionally one or more of 0-40 wt. % zinc, 0-10wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a beryllium-copper alloy includes 95-98.5 wt. % copper, 1-4 wt. % beryllium, and optionally one or more of 0-1wt. % cobalt, and 0-0.5 wt. % silicon. As defined herein, a titanium-nickel alloy (e.g., Nitinol alloy) includes 42-58 wt. % nickel and 42-58 wt. % titanium.

In accordance with another and/or alternative aspect of the present disclosure, the metal alloy that is used to partially or fully form the frame of the medical device includes at least 5 awt. % (e.g., 5-99 awt. % and all values and ranges therebetween) rhenium and/or hafnium, and 0.1-96 wt. % (and all values and ranges therebetween) of one or more additives selected from the group of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a rhenium and/or hafnium effect. In one non-limiting embodiment, the metal alloy that is used to partially or fully form the frame of the medical device is a stainless-steel alloy that has been modified to include at least 5-15 awt. % rhenium and/or hafnium. In another non-limiting embodiment, the metal alloy that is used to partially or fully form the frame of the medical device is a cobalt chromium alloy that has been modified to include at least 5-15 awt. % rhenium and/or hafnium. In another non-limiting embodiment, the metal alloy that is used to partially or fully form the frame of the medical device is a TiAlV alloy that has been modified to include at least 5-15 awt. % rhenium and/or hafnium. In another non-limiting embodiment, the metal alloy that is used to partially or fully form the frame of the medical device is an aluminum alloy that has been modified to include at least 5-15 awt. % rhenium and/or hafnium. In another non-limiting embodiment, the metal alloy that is used to partially or fully form the frame of the medical device is a nickel alloy that has been modified to include at least 5-15 awt. % rhenium and/or hafnium. In another non-limiting embodiment, the metal alloy that is used to partially or fully form the frame of the medical device is a titanium alloy that has been modified to include at least 5-15 awt. % rhenium and/or hafnium. In another non-limiting embodiment, the metal alloy that is used to partially or fully form the frame of the medical device is a tungsten alloy that has been modified to include at least 5-15 awt. % rhenium and/or hafnium. In another non-limiting embodiment, the metal alloy that is used to partially or fully form the frame of the medical device is a molybdenum alloy that has been modified to include at least 5-15 awt. % rhenium and/or hafnium. In another non-limiting embodiment, the metal alloy that is used to partially or fully form the frame of the medical device is a copper alloy that has been modified to include at least 5-15 awt. % rhenium and/or hafnium. In another non-limiting embodiment, the metal alloy that is used to partially or fully form the frame of the medical device is a beryllium-copper alloy that has been modified to include at least 5-15 awt. % rhenium and/or hafnium.

In another and/or alternative non-limiting aspect of the present disclosure, the metal alloy that is used to partially or fully formed the frame of the medical device includes less than about 5 wt. % (e.g., 0-4.999999 wt. % and all values and ranges therebetween) other metals and/or impurities, typically 0-1 wt. %, more typically 0-0.1 wt. %, even more typically 0-0.01 wt. %, and still even more typically 0-0.001 wt. %. A high purity level of the metal alloy can result in the formation of a more homogeneous alloy, which in turn can result in a more uniform density throughout the metal alloy, and also can result in the desired yield and ultimate tensile strengths of the metal alloy.

In accordance with another and/or alternative aspect of the present disclosure, the frame for a medical device is optionally subjected to one or more manufacturing processes. These manufacturing processes can include, but are not limited to, expansion, laser cutting, etching, crimping, annealing, drawing, pilgering, electroplating, electro-polishing, machining, plasma coating, 3D printed coatings, chemical vapor deposition, chemical polishing, cleaning, pickling, gun drilling, EDM cutting, CMC cutting, ion beam deposition or implantation, sputter coating, vacuum deposition, etc.

In accordance with another and/or alternative aspect of the present disclosure, the metal alloy optionally includes a certain amount of carbon and oxygen; however, this is not required. These two elements have been found to affect the forming properties and brittleness of the metal alloy. The controlled atomic ratio of carbon and oxygen of the metal alloy can also minimize the tendency of the metal alloy to form micro-cracks during the forming of the metal alloy into a frame for a medical device, and/or during the use and/or expansion of the frame for a medical device in a body. The carbon to oxygen atomic ratio can be as low as about 0.2:1 (e.g., 0.2:1 to 50:1 and all values and ranges therebetween). In one non-limiting formulation, the carbon to oxygen atomic ratio in the metal alloy is generally at least about 0.3:1. Typically the carbon content of the metal alloy is less than about 0.1 wt. % (e.g., 0-0.0999999 wt. % and all values and ranges therebetween), and more typically 0-0.01 wt. %. Carbon contents that are too large can adversely affect the physical properties of the metal alloy. Generally, the oxygen content is to be maintained at very low level. In one non-limiting formulation, the oxygen content is less than about 0.1 wt. % of the metal alloy (e.g., 0-0.0999999 wt. % and all values and ranges therebetween), and typically 0-0.01 wt. %.

In accordance with another and/or alternative aspect of the present disclosure, the metal alloy optionally includes a controlled amount of nitrogen; however, this is not required. Large amounts of nitrogen in the metal alloy can adversely affect the ductility of the metal alloy. This can in turn adversely affect the elongation properties of the metal alloy. In one non-limiting formulation, the metal alloy includes less than about 0.001 wt. % nitrogen (e.g., 0 wt. % to 0.0009999 wt. % and all values and ranges therebetween). It is believed that the nitrogen content should be less than the content of carbon or oxygen in the metal alloy. In one non-limiting formulation, the atomic ratio of carbon to nitrogen is at least about 1.5:1 (e.g., 1.5:1 to 400:1 and all values and ranges therebetween). In another non-limiting formulation, the atomic ratio of oxygen to nitrogen is at least about 1.2:1 (e.g., 1.2:1 to 150:1 and all value and ranges therebetween).

In accordance with another and/or alternative aspect of the present disclosure, the metal alloy that is used to form all or part of the frame for a medical device 1) is not clad, metal coated, metal sprayed, plated and/or formed (e.g., cold worked, hot worked, etc.) onto another metal, or 2) does not have another metal or metal alloy metal sprayed, coated, plated, clad and/or formed onto the metal alloy. It will be appreciated that in some applications, the metal alloy of the present disclosure may be clad, metal sprayed, coated, plated and/or formed onto another metal, or another metal or metal alloy may be plated, metal sprayed, coated, clad and/or formed onto the metal alloy when forming all or a portion of a frame for a medical device.

In accordance with another and/or alternative aspect of the present disclosure, the metal alloy can be used to form a) a coating (e.g., cladding, dip coating, spray coating, plated coating, welded coating, plasma coating, etc.) on a portion of all of a frame for a medical device, or b) a core of a portion or all of a frame for a medical device. The composition of the coating is different from the composition of the material surface to which the metal alloy is coated. The coating thickness of the metal alloy is non-limiting (e.g., 1 μm to 1 inch and all values and ranges therebetween). In one non-limiting example, there is provided a frame for a medical device wherein a core or base layer of the frame for a medical device is formed of a metal or metal alloy (e.g., chromium alloy, titanium, titanium alloy, stainless steel, iron alloy, CoCr alloy, rhenium alloy, molybdenum alloy, tungsten alloy, Ta-W alloy, refractory metal alloy, MoTa alloy, MoRe alloy, etc.) or polymer or ceramic or composite material, and the other layer of the coated frame for a medical device is formed of a different metal or metal alloy. The core or base layer and the other layer of the frame for a medical device can each form 10-99% (and all values and ranges therebetween) of the overall cross section of the frame for a medical device. When the outer metal coating is a rhenium and/or hafnium containing alloy, such rhenium and/or hafnium alloy can be used to create a hard surface on the frame for a medical device at specific locations as well as all over the surface. In another non-limiting embodiment, the core or base layer of the frame for a medical device can be formed of a rhenium and/or hafnium containing alloy and the coating layer includes one or more other materials (e.g., another type of metal or metal alloy [e.g., chromium alloy, titanium, titanium alloy, stainless steel, iron alloy, CoCr alloy, rhenium and/or hafnium alloy, molybdenum alloy, tungsten alloy, Ta-W alloy, refractory metal alloy, MoTa alloy, MoRe alloy, etc.), polymer coating, ceramic coating, composite material coating, etc.). Non-limiting benefits of using the rhenium and/or hafnium containing alloy in the core or interior layer of the frame for a medical device can include reducing the size of the frame for a medical device, increasing the strength of the frame for a medical device, and/or maintaining or reducing the cost of the frame for a medical device. As can be appreciated, the use of the rhenium and/or hafnium containing alloy can result in other or additional advantages. The core or base layer size and/or thickness of the metal alloy are non-limiting. In one non-limiting example, there is provided a frame for a medical device that is at least partially formed from layered materials wherein a top layer is formed of material that is different form one or more other layers and the rhenium and/or hafnium containing alloy forms one of the layers below the top layer, and the top layer is formed of a metal that is different from the rhenium and/or hafnium containing alloy (e.g., chromium alloy, titanium, titanium alloy, stainless steel, iron alloy, CoCr alloy, rhenium and/or hafnium alloy, molybdenum alloy, tungsten alloy, Ta-W alloy, refractory metal alloy, MoTa alloy, MoRe alloy, etc.). The core or lower layer or base layer and the outer layer of the layered material can each form 10-99% (and all values and ranges therebetween) of the overall cross section of the layered material.

In accordance with another and/or alternative aspect of the present disclosure, the metal alloy is optionally at least partially formed by a swaging process; however, this is not required. In one non-limiting embodiment, swaging is performed on the metal alloy to at least partially or fully achieve final dimensions of one or more portions of the frame for a medical device. The swaging dies can be shaped to fit the final dimension of the frame for a medical device; however, this is not required.

In accordance with another and/or alternative aspect of the present disclosure, the metal alloy can optionally be nitrided; however, this is not required. The nitrided layer on the metal alloy can function as a lubricating surface during the optional drawing of the metal alloy when partially or fully forming the frame for a medical device.

In another and/or alternative non-limiting aspect of the present disclosure, there is provided a medical device that is at least partially (e.g. 1-99.999 wt. % and all values and ranges therebetween) or fully formed of a material that is coated with an enhancement layer. In one non-limiting embodiment of the present disclosure, one or more components of the medical device (e.g., frame, inner skirt, outer skirt, leaflets, material used to secure leaflets to frame, etc.) can be partially (e.g., 1% to 99.99% and all values and ranges therebetween) or fully be coated with an enhancement layer to improve one or more properties of the medical device (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, reduce the rate of corrosion on the metal that forms the frame of the medical device, form a non-stick coated surface, improve biocompatibility of material having the coated surface, reduce toxicity of material having the coated surface, reduce ion release from material having the coated surface, reduced metal ion release of the metal material from the frame of the medical device, the enhancement layer forms a surface that is less of an irritant to cell about the coated surface after the medical device is implanted, reduces the rate of structural valve disease (SVD) or valve structural deterioration, reduces the rate to which cells grown on coated surface after medical device is implanted, reduce rate to which leaflets fail to properly operate after medical device is implanted, etc.). Medical device structural deterioration is due in part to the poor hemocompatibility, poor cytocompatibility, and the susceptibility of medical device components to excessive tissue proliferation, inflammatory response, and/or an unfavorable healing environment. In another non-limiting embodiment of the disclosure, medical device structural deterioration is inhibit or reduced by i) reducing neointimal hyperplasia/cell overgrowth onto one or more portions of the medical device after implantation in the treatment area, ii) reducing infection about the medical device after implantation in the treatment area, iii) reducing platelet activation about the medical device after implantation in the treatment area, iv) reducing thrombosis about the medical device after implantation in the treatment area, v) reducing restenosis about the medical device after implantation in the treatment area, vi) reducing the incidence of nickel exposure and/or ion release from the frame of the medical device that can react with cells about the medical device after implantation in the treatment area, vii) reducing inflammatory cell response about the medical device after implantation in the treatment area, and/or viii) promoting endothelial cell angiogenesis about the medical device after implantation in the treatment area. In another non-limiting embodiment, the enhancement layer on one or more portions of the medical device is formulated to provide and/or promote generation of nitric oxide near, at and/or in adjacent tissue. Nitric oxide can reduce neointimal hyperplasia, reduce tissue proliferation, reduce platelet activation, reduce thrombosis, reduce restenosis, and can promote endothelial cell angiogenesis, all of which can contribute to an improved pro-healing environment. In another non-limiting embodiment, the enhancement layer provides, promotes and/or facilitates in a) formation or generation of nitric oxide (NO), b) stimulation of endothelial cells, and/or c) a modulation of endothelial cells. In one non-limiting arrangement, there is provided a metal oxynitride layer that is deposited a portion or all of the medical device. For example, the metal oxynitride layer can be deposited on a portion or all of the outer surface of a) the frame of the medical device, b) the inner skirt of the medical device, c) the outer skirt of the medical device, and/or d) the one or more leaflets of the medical device. In one non-limiting specific configuration, the metal oxynitride layer is or includes titanium oxynitride and/or zirconium oxynitride. In another non-limiting specific configuration, the thickness of the metal oxynitride layer is at least 10 nanometers (e.g., 10 nanometers to 10 microns and all values and ranges therebetween). In one non-limiting specific configuration, the oxygen to nitrogen atomic ratios of the metal oxynitride layer is 1:10 to 10:1 (and all values and ranges therebetween). In another non-limiting specific configuration, the layer of metal oxynitride layer is optionally deposited onto a metallic adhesion layer in between the base substrate (e.g., frame, inner skirt, outer skirt, one or more leaflets, etc.) and the oxynitride layer, and wherein the adhesion layer optionally is or includes titanium metal and/or zirconium metal, and wherein the adhesion layer optionally has a thickness of 10 nanometers (e.g., 10 to 500 nanometers and all values and ranges therebetween). When the metal oxynitride layer is deposited on a portion or all of the outer surface of a) the frame of the medical device, b) the inner skirt of the medical device, c) the outer skirt of the medical device, and/or d) the one or more leaflets of the medical device, the metal oxynitride layer can be used to at least partially reduces the rate of structural valve disease (SVD) by i) having the outer surface of the deployed medical device frame that is partially or fully coated with the metal oxynitride layer to be at least partially in direct contact with the native endothelial cells of the heart valve, ii) having the outer surface of the deployed medical device outer skirt that is partially or fully coated with the metal oxynitride layer to be at least partially in direct contact with the native endothelial cells of the heart valve, iii) having the outer surface of the deployed medical device frame that is partially or fully coated with the metal oxynitride layer to be at least partially in direct contact with the native endothelial cells of the heart valve and the blood stream that flows through the heart valve, and/or iv) having the outer surface of the deployed medical device outer skirt that is partially or fully coated with the metal oxynitride layer to be at least partially in direct contact with the native endothelial cells of the heart valve and the blood stream that flows through the heart valve. Nitric oxide (NO) is a short-lived, gaseous, signal molecule responsible for a plurality of cellular functions throughout the human body. NO is endogenously biosynthesized from L-arginine, oxygen, and NADPH inputs via the Nitric Oxide Synthase enzyme family. In the cardiovascular system, NO acts as a potent vasodilator. NO is also involved in cellular repair during vascular damage. A primary effect of NO is binding to Soluble Guanylyl Cyclase (sGC) activating synthesis of a downstream signaling molecule Cyclic Guanosine Monophosphate (cGMP). cGMP is subsequently responsible for downregulation of factors responsible for platelet aggregation, apoptosis, inflammation, and tissue remodeling. cGMP is also responsible for upregulation of factors responsible for vasodilation. Examples of direct nitric oxide donors includes agents with nitroso or nitrosyl functional groups that spontaneously release nitric oxide. Examples of metabolic nitric oxide donors include agents with organic nitrate and nitrite esters requiring enzymatic metabolism to generate bioactive nitric oxide. Examples of bifunctional nitric oxide donors include agents with nitrate esters and S-nitrosothiols that release nitric oxide simultaneously with performing additional pharmacological benefits. The enhancement layer and/or the metallic adhesion layer can be applied by use of a vacuum coating process (e.g., physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process), plating process, etc.

In accordance with another and/or alternative non-limiting aspect of the present disclosure, one or more components of the medical device (e.g., frame, inner skirt, outer skirt, leaflets, material used to secure leaflets to frame, etc.) can be partially (e.g., 1% to 99.99% and all values and ranges therebetween) or fully be coated with an enhancement layer to improve one or more properties of the medical device. In one non-limiting embodiment, only the frame of the medical device includes the enhancement layer, and wherein the frame is partially (e.g., 1-99.99% and all values and ranges therebetween) or fully coated with the enhancement layer. In another non-limiting embodiment, only one or more of all of the leaflets of the medical device include the enhancement layer, and wherein one or more or all of the leaflets are partially (e.g., 1-99.99% and all values and ranges therebetween) or fully coated with the enhancement layer. In another non-limiting embodiment, only the inner skirt of the medical device includes the enhancement layer, and wherein the inner skirt is partially (e.g., 1-99.99% and all values and ranges therebetween) or fully coated with the enhancement layer. In another non-limiting embodiment, only the outer skirt of the medical device includes the enhancement layer, and wherein the outer skirt is partially (e.g., 1-99.99% and all values and ranges therebetween) or fully coated with the enhancement layer. In another non-limiting embodiment, two or more or all of a) the frame, b) one or more or all of the leaflets, c) the inner skirt and d) the outer skirt of the medical device are partially (e.g., 1-99.99% and all values and ranges therebetween) or are fully coated with the enhancement layer. Non-limiting enhancement layers that can be applied to a portion or all of the outer surface of one or more components of the medical device includes chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium oxynitride (ZrNxOy) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), and combinations of such coatings. In one non-limiting embodiment, the one or more enhancement layers are optionally applied to a portion or all of the outer surface of one or more components of the medical device by a vacuum process using an energy source to vaporize material and deposit a thin layer of enhancement layer material. Such vacuum coating process, when used, can include a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In one non-limiting embodiment, the coating process is one or more of a PVD, CVD, ALD and PE-CVD, and wherein the coating process occurs at a temperature of 200-400° C. (and all values and ranges therebetween) for at least 10 minutes (e.g., 10-400 minutes and all values and ranges therebetween). In another non-limiting embodiment, the coating process is one or more of a PVD, CVD, ALD and PE-CVD, and wherein the coating process occurs at a temperature of 220-300° C. for 60-120 minutes. In another non-limiting embodiment, when the materials of the one or more enhancement layers are to be applied to the outer surface of the frame of the medical device that is partially or fully formed of a metal alloy, the materials of the one or more enhancement layers can optionally be combine with one or more metals in the metal alloy, and/or combined with nitrogen, oxygen, carbon, or other elements that are in the metal alloy and/or present in the atmosphere about the metal alloy to a form an enhancement layer on the outer surface of the metal alloy. In another non-limiting embodiment, when the materials of the one or more enhancement layers are to be applied to the outer surface of the frame for a medical device that is partially or fully formed of a metal alloy, the materials of the one or more enhancement layers can optionally be used to form various coating colors on the outer surface of the metal alloy (e.g., gold, copper, brass, black, rose gold, chrome, blue, silver, yellow, green, etc.). In another non-limiting embodiment, the thickness of the enhancement layer is greater than 1 nanometer (e.g., 2 nanometers to 100 microns and all values and ranges therebetween), and typically 0.1-25 microns, and more typically 0.2-10 microns. In another non-limiting embodiment, the hardness of the enhancement layer can be at least 5 GPa (ASTM C1327-15 or ASTM C1624-05), typically 5-50 GPa (and all values and ranges therebetween), more typically 10-25 GPa, and still more typically 14-24 GPa. In another non-limiting embodiment, the coefficient of friction (COF) of the enhancement layer can be 0.04-0.2 (and all values and ranges therebetween), and typically 0.6-0.15. In another non-limiting embodiment, the wear rate of the enhancement layer can be 0.5×10⁻⁷ mm³/N-m to 3×10⁻⁷ mm³/N-m (an all values and ranges therebetween), and typically 1.2×10⁻⁷ mm³/N-m to 2×10⁻⁷ mm³/N-m. In another non-limiting embodiment, silicon-based precursors (e.g., trimethysilane, tetramethylsilane, hexachlorodisilane, silane, dichlorosilane, trichlorosilane, silicon tetrachloride, tris(dimethylamino) silane, bis(tert-butylamino)silane, trisilylamine, allyltrimethoxysilane, (3-aminopropyl)triethoxysilane, butyltrichlorosilane, n-sec-butyl(trimethylsilyl)amine, chloropentamethyldisilane, 1,2-dichlorotetramethyldisilane, [3-(diethylamino)propyl]trimethoxysilane, 1,3-diethyl-1,1,3,3-tetramethyldisilazane, dimethoxydimethylsilane, dodecamethylcyclohex asilane, hexamethyldisilane, isobutyl(trimethoxy)silane, methyltrichlorosilane, 2,4,6,8,10-pentamethylcyclopentasiloxane, pentamethyldisilane, n-propyltriethoxy silane, silicon tetrabromide, silicon tetrabromide, etc.) can optionally be used to facilitate in the application of the enhancement layer to one or more portions or all of the medical device. In one non-limiting embodiment, the enhancement layer includes no more than 0.1 wt. % nickel, no more than 0.1 wt. % chromium, and/or no more than 0.1 wt. % cobalt. In another non-limiting embodiment, the outer surface of the frame of the medical device includes no more than 0.1 wt. % nickel, no more than 0.1 wt. % chromium, and/or no more than 0.1 wt. % cobalt. The adhesion layer includes no more than 0.1 wt. % nickel, no more than 0.1 wt. % chromium, and/or no more than 0.1 wt. % cobalt.

In accordance with another and/or alternative non-limiting aspect of the present disclosure, one or more components of the medical device can be partially or fully coated with an enhancement layer composition that includes a chromium nitride (CrN) coating. A portion or all of the medical device can be partially or fully coated with the chromium nitride (CrN) coating. The enhancement layer can be used to improve hardness, improve toughness, reduced friction, resistant impact wear, improve resistance to corrosion and oxidation, and/or form a reduced stick surface when in contact with many different materials. In accordance with one non-limiting embodiment, the chromium nitride (CrN) coating generally includes 40-85 wt. % Cr (and all values and ranges therebetween), 15-60 wt. % N (and all values and ranges therebetween), 0-10 wt. % Re (and all values and ranges therebetween), 0-10 wt. % Si (and all values and ranges therebetween), 0-2 wt. % O (and all values and ranges therebetween), and 0-2 wt. % C (and all values and ranges therebetween). In one non-limiting coating process, all or a portion of the outer surface of one or more components of the medical device are initially coated with Cr metal. The Cr metal coating can be applied by PVD, CVD, ALD and PE-CVD in an inert environment. The coating thickness of Cr metal is 0.5-15 microns. Thereafter, the Cr metal coating is exposed to nitrogen gas and/or a nitrogen containing gas compound to cause the nitrogen to react with the Cr metal coating to form a layer of CrN on the outer surface of the Cr metal coating and/or the outer surface of one or more components of the medical device. Particles of Cr metal can optionally be mixed with nitrogen gas and/or a nitrogen containing gas compound to facilitate in the formation of the CrN coating. When Cr metal particles are used, the initial Cr coating layer on the outer surface of one or more components of the medical device can optionally be eliminated. In another non-limiting embodiment, the enhancement layer composition generally includes 65-80 wt. % Cr, 15-30 wt. % N, 0-8 wt. % Re, 0-1 wt. % Si, 0-1 wt. % O, and 0-1 wt. % C.

In accordance with another and/or alternative non-limiting aspect of the present disclosure, one or more components of the medical device can be partially or fully coated with an enhancement layer composition that includes a diamond-Like Carbon (DLC) coating. A portion or all of the medical device can be partially or fully coated with the diamond-Like Carbon (DLC) coating. The enhancement layer can be used to improve hardness, improve toughness, reduced friction, resistant impact wear, improve resistance to corrosion and oxidation, improve biocompatibility, and/or form a reduced stick surface when in contact with many different materials. In one non-limiting embodiment, the diamond-Like Carbon (DLC) coating generally includes 60-99.99 wt. % C (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % O (and all values and ranges therebetween). The carbon coating can be applied by PVD, CVD, ALD and PE-CVD in an inert environment. The carbon layer can be applied by using methane and/or acetylene gas; however, other or additional carbon sources can be used. The coating thickness of the carbon is 0.5-15 microns. In another non-limiting embodiment, all or a portion of the medical device are coated with the enhancement layer composition that generally includes 90-99.99 wt. % C, 0-1 wt. % N, 0-8 wt. % Re, 0-1 wt. % Si, and 0-1 wt. % O.

In accordance with another and/or alternative non-limiting aspect of the present disclosure, one or more components of the medical device can be partially or fully coated with an enhancement layer composition that includes a titanium nitride (TiN) coating. A portion or all of the outer surface of the one or more components of the medical device can include the titanium nitride (TiN) coating. The enhancement layer can be used to improve hardness, improve toughness, improve resistance to corrosion and oxidation, reduced friction, and/or form a reduced stick surface when in contact with many different materials. In one non-limiting embodiment, all or a portion of the outer surface of the one or more components of the medical device are optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating, when applied, is exposed to nitrogen gas and/or a nitrogen containing gas compound and optionally titanium particles to cause the nitrogen to react with the Ti metal coating and/or titanium metal particles to form a layer of TiN on the outer surface of the Ti metal coating and/or the outer surface of the one or more components of the medical device. If a titanium layer is not preapplied, the TiN coating can be formed by exposing the outer surface of one or more components of the medical device to titanium particles and nitrogen gas and/or a nitrogen containing gas compound. The coating thickness of the TiN coating is generally at least 0.1 microns (e.g., 0.1-15 microns and all values and ranges therebetween), and typically 0.2-2 microns.

In accordance with another and/or alternative non-limiting aspect of the present disclosure, one or more components of the medical device can be partially or fully coated with an enhancement layer composition that includes a titanium oxynitride or titanium nitride oxide (TiNOx) coating. A portion or all of the outer surface of the one or more components of the medical device can include the titanium oxynitride or titanium nitride oxide (TiNOx) coating. The enhancement layer can be used to improve hardness, improve toughness, improve resistance to corrosion and oxidation, reduced friction, and/or form a reduced stick surface when in contact with many different materials, and/or promote nitric oxide formation on the surface of the coating. In one non-limiting embodiment, all or a portion of the outer surface of the one or more components of the medical device are optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating is exposed to titanium particles and a nitrogen and oxygen mixture that can include nitrogen gas, oxygen gas, a nitrogen containing gas compound and/or an oxygen containing gas compound to cause the nitrogen and oxygen to react with the Ti metal coating, if such coating is used, and/or with the Ti metal particles to form a layer of TiNOx on the outer surface of the Ti metal coating and/or the outer surface of one or more components of the medical device. The ratio of the N to the O can be varied to control the amount of O in the TiNOx coating. If a titanium layer is not preapplied, the TiNOx coating can be formed by exposing one or more components of the medical device to titanium particles and a nitrogen and oxygen source such as nitrogen gas, oxygen gas, a nitrogen containing gas compound and/or an oxygen containing gas compound. The ratio of N to O when forming the TiNOx coating is generally 1:10 to 10:1 (and all values and ranges therebetween). The coating thickness of the TiNOx coating is generally at least 0.1 microns (e.g., 0.1-15 microns and all values and ranges therebetween), and typically 0.2-2 microns. In another non-limiting embodiment, a TiNOx coating is applied to a portion or all of the outer surface of the one or more components of the medical device, and the TiNOx coating is formed by a) exposing the outer surface of a portion of all of the one or more components of the medical device to Ti particles (PVD, CVD, ALD and PE-CVD process) and/or a Ti containing solution to form a Ti layer on a portion of all of the one or more components of the medical device, and wherein the thickness of the Ti coating is 0.05-5 microns, and b) exposing the Ti coating to a nitrogen and oxygen source such as nitrogen gas, oxygen gas, a nitrogen containing gas compound and/or an oxygen containing gas compound to form a TiNOx coating, and wherein ratio of N to O when forming the TiNOx coating is generally 1:10 to 10:1, and wherein the coating thickness of the TiNOx coating is 0.2 5 microns. In another non-limiting embodiment, a TiNOx coating is applied to a portion or all of the outer surface of the one or more components of the medical device, and the TiNOx coating is formed by exposing a portion or all of the outer surface of the medical device to Ti particles and a nitrogen and oxygen source such as nitrogen gas, oxygen gas, a nitrogen containing gas compound and/or an oxygen containing gas compound to form a TiNOx coating, and wherein ratio of N to O when forming the TiNOx coating is generally 1:10 to 10:1, and wherein the coating thickness of the TiNOx coating is 0.2-5 microns. In another non-limiting embodiment, the enhancement layer composition generally includes 20-85 wt. % Ti (and all values and ranges therebetween), 0.5-35 wt. % N (and all values and ranges therebetween), 0-10 wt. % Re (and all values and ranges therebetween), and 0.5-35 wt. % O (and all values and ranges therebetween). In another non-limiting embodiment, a coating of TiNOx was formed on one or more components of the medical device by reactive physical vapor deposition in a vacuum chamber. Depending on the oxygen-nitrogen ratio during vapor deposition, a coating deposit of TiNOx with defined composition and resistivity can be coated on the outer surface of the one or more components of the medical device.

In accordance with another and/or alternative non-limiting aspect of the present disclosure, the medical device can be partially or fully coated with an enhancement layer composition that includes a zirconium nitride (ZrN) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the one or more components of the medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound to cause the nitrogen to react with the Zn metal coating to form a layer of ZrN on the outer surface of the Zr metal coating and/or the outer surface of one or more components of the medical device. Particles of Zr metal can optionally be mixed with nitrogen gas and/or a nitrogen containing gas compound to facilitate in the formation of the ZrN coating. When Zr metal particles are used, the initial Zr coating layer on the outer surface of one or more components of the medical device can optionally be eliminated. The ZrN coating has been found to produce a gold-colored enhancement layer color. In another non-limiting embodiment, the enhancement layer composition generally includes 35-90 wt. % Zr (and all values and ranges therebetween), 5-25 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another non-limiting embodiment, the enhancement layer composition generally includes 80-90 wt. % Zr, 10-20 wt. % N, 0-8 wt. % Re, 0-1 wt. % Si, 0-1 wt. % O, and 0-1 wt. % C.

In accordance with another and/or alternative non-limiting aspect of the present disclosure, the medical device can be partially or fully coated with an enhancement layer composition that includes a zirconium oxide (ZrO₂) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the one or more components of the medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to oxygen gas and/or oxygen containing gas compound to cause the oxygen to react with the Zn metal coating to form a layer of zirconium oxide (ZrO₂) on the outer surface of the Zr metal coating and/or the outer surface of the one or more components of the medical device. Particles of Zr metal can optionally be mixed with oxygen gas and/or an oxygen containing gas compound to facilitate in the formation of the ZrO₂ coating. When Zr metal particles are used, the initial Zr coating layer on the outer surface of one or more components of the medical device can optionally be eliminated. The zirconium oxide (ZrO₂) coating has been found to produce a blue colored enhancement layer color. In another non-limiting embodiment, the enhancement layer composition generally includes 35-90 wt. % Zr (and all values and ranges therebetween), 10-35 wt. % O (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % C (and all values and ranges therebetween). In another non-limiting embodiment, the enhancement layer composition generally includes 70-80 wt. % Zr, 20-30 wt. %, 0-1 wt. % N, 0-8 wt. % Re, 0-1 wt. % Si, and 0-1 wt. % C.

In accordance with another and/or alternative non-limiting aspect of the present disclosure, the medical device can be partially or fully coated with an enhancement layer composition that includes both a zirconium oxide (ZrO₂) coating and a zirconium nitride coating (ZrN). The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both oxygen gas and/or oxygen containing gas compound and also to nitrogen gas and/or nitrogen containing gas compound, b) nitrogen gas and/or nitrogen containing gas compound and then to oxygen gas and/or oxygen containing gas compound, or c) oxygen gas and/or oxygen gas containing compound and then to nitrogen gas and/or nitrogen gas containing compound. The coating composition of the zirconium oxide (ZrO₂) coating and the zirconium nitride coating (ZrN) are similar or the same as discussed above. As discussed above, Particles of Zr metal can optionally be mixed with oxygen gas and/or an oxygen containing gas compound to facilitate in the formation of the ZrO₂ coating and the nitrogen gas and/or nitrogen gas containing compound to facilitate in the formation of the ZrN coating. When Zr metal particles are used, the initial Zr coating layer on the outer surface of one or more components of the medical device can optionally be eliminated.

In accordance with another and/or alternative non-limiting aspect of the present disclosure, the medical device can be partially or fully coated with an enhancement layer composition that includes a zirconium oxycarbide (ZrOC) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both to oxygen gas and/or an oxygen containing gas compound and to carbon and/or a carbon containing gas compound (e.g., methane and/or acetylene gas), b) carbon and/or a carbon containing gas compound and then to oxygen gas and/or an oxygen containing gas compound, or c) oxygen gas and/or oxygen containing gas compound and then to carbon and/or carbon containing gas compound. Particles of Zr metal can optionally be mixed with oxygen gas and/or an oxygen containing gas compound and the carbon and/or carbon containing gas compound to facilitate in the formation of the zirconium oxycarbide (ZrOC) coating. When Zr metal particles are used, the initial Zr coating layer on the outer surface of one or more components of the medical device can optionally be eliminated. In another non-limiting embodiment, the enhancement layer composition generally includes 40-95 wt. % Zr (and all values and ranges therebetween), 5-25 wt. % O (and all values and ranges therebetween), and 10-40 wt. % C (and all values and ranges therebetween), 0-2 wt. % N (and all values and ranges therebetween), 0-10 wt. % Re (and all values and ranges therebetween), and 0-20 wt. % Si (and all values and ranges therebetween). In another non-limiting embodiment, the enhancement layer composition generally includes 40-65 wt. % Zr, 5-25 wt. % O, and 25-40 wt. % C, 0-1 wt. % N, 0-8 wt. % Re, and 0-1 wt. % Si.

In accordance with another and/or alternative non-limiting aspect of the present disclosure, one or more components of the medical device can be partially or fully coated with an enhancement layer composition that includes a zirconium oxynitride (ZrNxOy) [e.g., cubic ZrN:O, cubic ZrO₂:N, tetragonal ZrO₂:N, and monoclinic ZrO₂:N phase coatings]. A portion or all of the outer surface of the one or more components of the medical device can include the zirconium oxynitride (ZrNxOy). The enhancement layer can be used to improve hardness, improve toughness, improve resistance to corrosion and oxidation, reduced friction, form a reduced stick surface when in contact with many different materials, and/or promote nitric oxide formation on the surface of the coating. In one non-limiting embodiment, all or a portion of the outer surface of the one or more components of the medical device are optionally initially coated with Zr metal. The Zr metal coating, when applied, can be applied by PVD, CVD, ALD and PE-CVD in an inert environment. The coating thickness of Zr metal is 0.05-15 microns (and all values and ranges therebetween). As can be appreciated, the initial Zr coating is optional. Thereafter, the Zr metal coating is exposed to zirconium particles and a nitrogen and oxygen mixture that can include nitrogen gas, oxygen gas, a nitrogen containing gas compound and/or an oxygen containing gas compound to cause the nitrogen and oxygen to react with the Zr metal coating, if such coating is used, and/or with the Zr metal particles to form a layer of ZrNxOy on the outer surface of the Zr metal coating and/or the outer surface of the one or more components of the medical device. The ratio of the N to the O can be varied to control the amount of O and N in the ZrNxOy coating. If a zirconium layer is not preapplied, the ZrNxOy coating can be formed by exposing the outer surface of one or more components of the medical device to zirconium particles and a nitrogen and oxygen source such as nitrogen gas, oxygen gas, a nitrogen containing gas compound and/or an oxygen containing gas compound. The ratio of N to O when forming the ZrNxOy coating is generally 1:10 to 10:1 (and all values and ranges therebetween). The coating thickness of the ZrNxOy coating is generally at least 0.1 microns (e.g., 0.1-15 microns and all values and ranges therebetween), and typically 0.2-2 microns. In another non-limiting embodiment, a ZrNxOy coating is applied to a portion or all of the outer surface of the one or more components of the medical device, and the ZrNxOy coating is formed by a) exposing the outer surface of a portion of all of the one or more components of the medical device to Zr particles (PVD, CVD, ALD and PE-CVD process) and/or a Zr containing solution to form a Zr layer on a portion of all of the one or more components of the medical device, and wherein the thickness of the Zr coating is 0.05-5 microns, and b) exposing the Zr coating to a nitrogen and oxygen source such as nitrogen gas, oxygen gas, a nitrogen containing gas compound and/or an oxygen containing gas compound to form a ZrNxOy coating, and wherein ratio of N to O when forming the ZrNxOy coating is generally 1:10 to 10:1, and wherein the coating thickness of the ZrNxOy coating is 0.2-5 microns. In another non-limiting embodiment, a ZrNxOy coating is applied to a portion or all of the outer surface of the one or more components of the medical device, and the ZrNxOy coating is formed by exposing a portion or all of the outer surface of the one or more components of the medical device to Zr particles and a nitrogen and oxygen source such as nitrogen gas, oxygen gas, a nitrogen containing gas compound and/or an oxygen containing gas compound to form a ZrNxOy coating, and wherein ratio of N to O when forming the ZrNxOy coating is generally 1:10 to 10:1, and wherein the coating thickness of the ZrNxOy coating is 0.2-5 microns. In another non-limiting embodiment, the enhancement layer composition generally includes 20-85 wt. % Zr (and all values and ranges therebetween), 0.5-35 wt. % N (and all values and ranges therebetween), and 0.5-35 wt. % O (and all values and ranges therebetween). In another non-limiting embodiment, a coating of ZrNxOy was formed on one or more components of the medical device by reactive physical vapor deposition in a vacuum chamber. Depending on the oxygen-nitrogen ratio during vapor deposition, a coating deposit of ZrNxOy with defined composition and resistivity can be coated on the outer surface of the one or more components of the medical device.

In accordance with another and/or alternative non-limiting aspect of the present disclosure, the medical device can be partially or fully coated with an enhancement layer composition that includes a zirconium-nitrogen-carbon (ZrNC) coating. The enhancement layer can be used to improve hardness, improve toughness, improve resistance to corrosion and oxidation, reduced friction, and/or form a reduced stick surface when in contact with many different materials. In one non-limiting embodiment all or a portion of the outer surface of the one or more components of the medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound and then to carbon and/or a carbon containing gas compound (e.g., methane and/or acetylene gas). The color of the ZrNC will vary depending on the amount of C and N in the coating. Particles of Zr metal can optionally be mixed with nitrogen gas and/or a nitrogen containing gas compound and the carbon and/or a carbon containing gas compound to facilitate in the formation of the ZrNC coating. When Zr metal particles are used, the initial Zr coating layer on the outer surface of one or more components of the medical device can optionally be eliminated. In one non-limiting embodiment, the enhancement layer composition generally includes 40-95 wt. % Zr (and all values and ranges therebetween), 5-40 wt. % N (and all values and ranges therebetween), and 5-40 wt. % C (and all values and ranges therebetween), 0-2 wt. % O (and all values and ranges therebetween), 0-10 wt. % Re (and all values and ranges therebetween), and 0-20 wt. % Si (and all values and ranges therebetween). In another non-limiting embodiment, the enhancement layer composition generally includes 40-80 wt. % Zr, 5-25 wt. % N, and 5-25 wt. % C, 0-1 wt. % O, 0-8 wt. % Re, and 0-1 wt. % Si.

In accordance with another and/or alternative non-limiting aspect of the present disclosure, the frame for a medical device can optionally be partially (e.g., 1% to 99.99% and all values and ranges therebetween) or fully be coated with and/or include one or more agents, which one or more agents are different in composition from the one or more enhancement layers, when such one or more enhancement layers are included on the medical device. As defined herein, a coating of an agent is not an enhancement coating When one or more agents are coated on the medical device, and the medical device includes an enhancement layer, one or more agents are generally coated on the outer surface of the enhancement layer. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungus and/or bacterial infection; vascular diseases and/or disorders; lymphatic diseases and/or disorders; cancer; implant rejection; pain; nausea; swelling; organ failure; immunity diseases and/or disorders; cell growth inhibitors, blood diseases and/or disorders; heart diseases and/or disorders; neuralgia diseases and/or disorders; fatigue; genetic diseases and/or disorders; trauma; cramps; muscle spasms; tissue repair; nerve repair; neural regeneration and/or the like.

The type and/or amount of agent included coated on frame for a medical device can vary. In accordance with another and/or alternative aspect of the present disclosure, one or more portions of the frame for a medical device can optionally 1) include the same or different agents, 2) include the same or different amount of one or more agents, 3) include the same or different polymer coatings, 4) include the same or different coating thicknesses of one or more polymer coatings, 5) have one or more portions of the frame for a medical device controllably release and/or uncontrollably release one or more agents, and/or 6) have one or more portions of the frame for a medical device controllably release one or more agents and one or more portions of the frame for a medical device uncontrollably release one or more agents.

In accordance with another and/or alternative aspect of the present disclosure, one or more surfaces of the frame of the medical device can optionally be treated. Such surface treatment techniques include, but are not limited to, cleaning, buffing, smoothing, nitriding, annealing, swaging, cold working, etching (chemical etching, plasma etching, etc.), etc. As can be appreciated, other or additional surface treatment processes can be used prior to the coating of one or more agents and/or polymers on the surface of the frame for a medical device.

In accordance with another and/or alternative non-limiting aspect of the present disclosure, when one or more agents are coated on one or more components of the medical device, and one or more components of the medical device includes an enhancement layer, such one or more agents are generally coated on the outer surface of the enhancement layer. If one or more components of the medical device are absent an enhancement layer, such one or more agents can be directly applied to the outer surface of the one or more components of the medical device are absent an enhancement layer. In accordance with another and/or alternative non-limiting aspect of the present disclosure, one or more portion of the medical device (e.g., frame, etc.) can optionally include a marker material that facilitates enabling the medical device to be properly positioned in the treatment area of the heart. In accordance with another and/or alternative non-limiting aspect of the present disclosure, one or more portions of the structures can be at least partially formed by MEMS (e.g., micro-machining, etc.) technology and/or other types of technology (e.g., 3D printing, etc.).

In another and/or alternative non-limiting aspect of the disclosure, the frame for a medical device can optionally include a marker material that facilitates enabling the frame for a medical device to be properly positioned in a body passageway (e.g., heart, kidney, blood vessel, urethra, ureters, and other body passageways). The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.).

In accordance with another and/or alternative aspect of the present disclosure, the frame for a medical device or one or more regions of the frame for a medical device can optionally be constructed by use of one or more microelectromechanical manufacturing (MEMS) techniques (e.g., micro-machining, laser micro-machining, micro-molding, etc.); however, other or additional manufacturing techniques can be used.

In accordance with another and/or alternative aspect of the present disclosure, the frame for a medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology.

In accordance with another and/or alternative aspect of the present disclosure, the frame for a medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie like structure, etc.) on the surface of the frame for a medical device. As defined herein, a “micro-structure” is a structure having at least one dimension (e.g., average width, average diameter, average height, average length, average depth, etc.) that is no more than about 2 mm, and typically no more than about 1 mm. Non-limiting examples of structures that can be formed on the one or more portions of the medical device are illustrated in U.S. Pat. Nos. 7,255,710 and 7,141,063, which are incorporated herein by reference.

In accordance with another and/or alternative non-limiting aspect of the present disclosure, the frame of the medical device can optionally be an expandable device that can be expanded by use of some other device (e.g., balloon, etc.).

In accordance with another and/or alternative non-limiting aspect of the present disclosure, the frame of the medical device can optionally be fabricated from a material having no or substantially no shape-memory characteristics.

In accordance with another and/or alternative non-limiting aspect of the present disclosure, the frame of the medical device can optionally be fabricated from a material having shape-memory characteristics.

In accordance with another and/or alternative aspect of the present disclosure, there is optionally provided a near net process for a frame of the frame for a medical device. In one non-limiting embodiment of the disclosure, there is provided a method of pressing together powder metal alloy and then sintering the compressed metal alloy powder to form a green part. The green part can be subsequently worked (e.g., cold worked), cut, coated, etc. to partially or fully form the metal device. In one non-limiting embodiment, the green part is pressed and then sintered. Thereafter, the sintered part is again pressed to increase its mechanical strength by imparting cold work into the pressed and sintered part.

In accordance with another and/or alternative aspect of the present disclosure, the metal alloy used to at least partially or fully form the frame for a medical device can optionally be initially formed into a blank, a rod, a tube, etc., and then finished into final form by one or more finishing processes. The metal alloy blank, rod, tube, etc., can be formed by various techniques such as, but not limited to, 1) melting the metal alloy and/or metals that form the metal alloy (e.g., vacuum arc melting, etc.) and then extruding and/or casting the metal alloy into a blank, rod, tube, etc., 2) melting the metal alloy and/or metals that form the metal alloy, forming a metal strip, and then rolling and welding the strip into a blank, rod, tube, etc., 3) consolidating the metal powder of the metal alloy and/or metal powder of metals that form the metal alloy into a blank, rod, tube, etc., or 4) 3-D printing the metal powder of the metal alloy and/or metal powder of metals that form the metal alloy into a blank, rod, tube, etc. When the metal alloy is formed into a blank, the shape and size of the blank is non-limiting. The rod can be optionally gun drilled or cut to form a tube.

In accordance with another and/or alternative aspect of the present disclosure, when the metal powder is consolidated to form the metal alloy into a blank, rod, tube, etc., the metal powder is pressed together to form a solid solution of the metal alloy into a near net frame for a medical device, near net component of a frame for a medical device, blank, rod, tube, etc. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.), and/or under a vacuum; however, this is not required. Generally, after the metal alloy powdered is pressed together, the pressed together metal powder is sintered.

In accordance with another and/or alternative aspect of the present disclosure, when metal powder is used to 3D print a portion or all of the frame for a medical device, component of a frame for a medical device, blank, rod, tube, etc., the average particle size of the metal powder is optionally 2-62 microns, and more particularly about 5-49.9 microns, the average density of the metal powders is greater than 5 g/cm³, and the metal powder is generally spherical-shaped, and the Hall flow (s/50 g) is less than 30 seconds (e.g., 2-29.99 seconds and all values and ranges therebetween). In another non-limiting embodiment of the disclosure, the average tensile elongation of the metal alloy used to partially or fully form the medical device is optionally at least about 25% (e.g., 25%-50% average tensile elongation and all values and ranges therebetween). This same sized metal powder can be used when forming a green part as discussed above.

In accordance with another and/or alternative non-limiting aspect of the present disclosure, the expandable frame of the medical device can be configured to be crimped to a diameter of less than 24 FR (e.g., less than 8 mm, 5-7.9 mm, etc.) and the expandable frame and/or medical device can be configured to be expanded to a diameter of at least 14 mm (e.g., 14-35 mm and all values and ranges therebetween); however, it can be appreciated that the expandable frame and/or medical device can be designed to be crimped to larger diameters, and/or be expanded to larger diameters.

In accordance with another and/or alternative non-limiting aspect of the present disclosure, the frame has 0-20% longitudinal foreshortening (and all values and ranges therebetween) along a longitudinal axis of the expandable frame when the expandable frame is plastically deformed (e.g., expanded from the crimped to the uncrimped position).

In accordance with another and/or alternative non-limiting aspect of the present disclosure, the material used to form the expandable metal frame contains little or no nickel (e.g., 0-2 wt. % nickel and all values and ranges therebetween).

In accordance with another and/or alternative non-limiting aspect of the present disclosure, the expandable frame can include distinguishing features which allows for rotational alignment of the expandable frame with the commissures of the native heart valve. In one non-limiting embodiment, the one or more distinguishing feature on the expandable heart valve are asymmetrical for identification of rotational alignment of the expandable frame. In another non-limiting embodiment, the one or more distinguishing features are attached directly to the commissure of the medical device frame. In another non-limiting embodiment, the one or more the distinguishing feature can be formed of radiopaque material which allows for high visibility during the insertion procedure of the medical device in the heart.

In accordance with another and/or alternative non-limiting aspect of the present disclosure, the expandable frame of the medical device can be optionally coated with a polymer material (e.g., silicone, PTFE, ePTFE, polyurethane, polyolefins, hydrogels, biological materials (e.g., pericardium or biological polymers such as collagen, gelatin, or hyaluronic acid derivatives), etc.). The coating can be used to partially or fully encapsulate the angular articulating members and/or vertically extending axial longitudinal members on the frame and/or to fill-in the openings between the angular articulating members and/or vertically extending axial longitudinal members on the frame.

In accordance with another and/or alternative non-limiting aspect of the present disclosure, the medical device includes a radially collapsible and expandable frame that includes one or more frame opening arrangements that can optionally be used as securing locations for the one of more leaflet structures, leaflet, inner skirt, and/or outer skirt. In one non-limiting embodiment, one or more of the frame opening arrangements includes a first and optionally a second frame opening. The size and shape of the lower frame opening and optional an upper frame opening are non-limiting. In one non-limiting configuration, the one or more optional upper frame openings can be used as a marker to facilitate in the proper positioning of the frame and medical device in the heart.

In accordance with another and/or alternative non-limiting aspect of the present disclosure, the medical device includes a radially collapsible and expandable frame that includes one or more of the following features: a) high radial strength after expansion of the frame, b) small crimp profile; c) use of a material that minimal recoil after expansion of the frame; d) little or no longitudinal foreshortening of the frame during expansion; e) smooth curvature at peaks and along angular articulating members and/or the axial longitudinal members of the frame; f) symmetrical design for restoration of valve function and visualization of frame; g) markers on frame for commissural alignment; and/or h) open cell aligned with coronary for hemodynamic and reintervention.

One non-limiting object of the present disclosure is the provision of a delivery system for a medical device that includes an integrated filter arrangement that has one or more of the following features: a) captures, not merely deflects, embolic material that has dislodged from the heart during a medical device procedure, b) protects from dislodged embolic material entering the right hemisphere and left anterior circulation to the patient's brain, and also protects from dislodged embolic material the left posterior circulation of the brain and other arteries from the heart to the patient (kidneys, liver, legs, etc.), c) does not require additional blood vessel access during the deployment of the medical device, thus not increasing the potential for vascular access complications, d) does not interfere with the TAVR valve as the TAVR valve crosses the aortic arch, e) is integrated with the heart valve delivery system and is located in-line with the longitudinal axis of the catheter portion that includes the medical device, f) has a self-expanding filter frame (e.g., filter frame is partially or fully formed of a shape memory material, frame includes a biasing arrangement to bias the frame in the open position, etc.), g) the filter arrangement can be easily constrained in a collapsed position by the delivery system, can be easily unconstrained and deployed by the delivery system prior to the heart valve being expanded in treatment area of the heart, and can be easily be again collapsed and constrained by the delivery system after the heart valve has been deployed, and/or h) removes captured material from the patient when the filter arrangement is removed from the patient.

Another and/or alternative non-limiting object of the present disclosure is the provision of a vascular delivery device arrangement comprising a delivery system and a filter arrangement; said delivery system includes a catheter and a guide shaft; said catheter and said guide shaft are configured to move independently from one another; said filter arrangement is connected to said catheter such that at least a portion or all of the filter arrangement is located proximal to said cardiovascular implant; said filter arrangement is configured to move between a constrained-collapsed position and a deployed position; said filter arrangement includes a filter frame and a filter material; said guide shaft is moveable relative to said filter arrangement between a constraining and non-constraining position; said filter arrangement is positioned in said constrained-collapsed position when said guide shaft is in said constraining position due to said guide shaft being positioned partially or fully over said filter arrangement; said filter arrangement is moveable to said deployed position when said guide shaft is in said non-constraining position due to said guide shaft being positioned partially or fully proximal to said filter arrangement.

Another and/or alternative non-limiting object of the present disclosure is the provision of a vascular delivery device arrangement wherein said catheter includes a balloon catheter and wherein an expandable medical device can be positioned at least partially over a balloon on said catheter.

Another and/or alternative non-limiting object of the present disclosure is the provision of a vascular delivery device arrangement wherein said filter frame includes shape memory material.

Another and/or alternative non-limiting object of the present disclosure is the provision of a vascular delivery device arrangement wherein said catheter is a coaxial catheter and said filter arrangement is positioned at least partially on and/or within one or more layers of said coaxial catheter.

Another and/or alternative non-limiting object of the present disclosure is the provision of a vascular delivery device arrangement wherein said filter material is configured to filter particles having a size of 450 microns and greater.

Another and/or alternative non-limiting object of the present disclosure is the provision of a vascular delivery device arrangement wherein said filter material is configured to allow at least 0.25 L/min. of fluid flow through said filter material.

Another and/or alternative non-limiting object of the present disclosure is the provision of a vascular delivery device arrangement wherein said filter material is at least partially polymeric and/or metallic.

Another and/or alternative non-limiting object of the present disclosure is the provision of a vascular delivery device arrangement wherein said filter material is at least partially formed of polyurethane, polyethylene terephthalate, polystyrene, polypropylene, UHM (Ultra-high-molecular-weight) polyethylene polymers, nylon, nitinol, shape memory material and/or metallic film.

Another and/or alternative non-limiting object of the present disclosure is the provision of a vascular delivery device arrangement wherein said filter is configured to cause said filter material to contact a vasculature wall so that at least a portion of fluid flows through said filter material when said filter arrangement is in said deployed position.

Another and/or alternative non-limiting object of the present disclosure is the provision of a vascular delivery device arrangement wherein said filter frame forms an umbrella-shaped filter arrangement, conical-shaped filter arrangement, or a clamshell-shaped filter arrangement.

Another and/or alternative non-limiting object of the present disclosure is the provision of a vascular delivery device arrangement wherein said filter frame includes two or more longitudinal members that are connected to said catheter.

Another and/or alternative non-limiting object of the present disclosure is the provision of a vascular delivery device arrangement wherein said filter frame contains one or more self-expanding loops.

Another and/or alternative non-limiting object of the present disclosure is the provision of a vascular delivery device arrangement wherein said vascular delivery device arrangement is configured to deliver a medical device to a treatment site; said medical device is selected from the group consisting of a heart valve, a stent, an expandable balloon, and a medical device having an expandable frame.

Another and/or alternative non-limiting object of the present disclosure is the provision of a method for deploying a medical device at a treatment site in a vascular system or other body passageway comprising: a) providing a medical device; said medical device is selected from the group consisting of a heart valve, a stent, an expandable balloon, and a medical device having an expandable frame; b) providing a device delivery arrangement as defined in claim 1, wherein said device delivery arrangement is configured to deliver said medical device to said treatment site; c) folding said filter arrangement to said constrained-collapsed position; d) moving said guide shaft over at least a portion of said filter arrangement to maintain said filter arrangement in said constrained-collapsed position; e) advancing said catheter through a patient's anatomy until said medical device is positioned at said treatment site while both said medical device is in an unexpanded position and said filter arrangement is in said constrained-collapsed position; f) moving said guide shaft partially or fully off of said filter arrangement to enable said filter arrangement to move to said deployed position; g) expanding said medical device at said treatment site while said filter arrangement is positioned in said deployed position; h) moving said guide shaft over at least a portion or all of said filter arrangement to cause said filter arrangement to move to said constrained-collapsed position; and i) removing said device delivery arrangement from said patient; and wherein said filter arrangement is configured to capture at least a portion of dislodged emboli that has dislodge due to said expansion of said medical device at said treatment area.

Another and/or alternative non-limiting object of the present disclosure is the provision of a medical device that includes a frame that has one or more of the following: a) optionally has a novel geometry, b) a frame that optionally is partially or fully formed of a rhenium and/or hafnium containing alloy, c) has an open cell geometry in the frame of the medical device that can be used to reduce delivery system size reducing vascular and neurological complications, d) has high radial strength using an open cell pattern due to the high yield strength and ultimate tensile strength of the metal alloy, e) has improved restoration of the physiologic EOA in challenging, heavily calcified valves that exert high force on the bioprosthetic valve, while allowing a reduced crimp diameter for vascular access, f) has improved restoration of the physiologic EOA that results in greater longevity of the bioprosthetic valve, g) is partially or fully formed of a material having lower recoil than the traditional materials of stainless steel, chromium-cobalt, or titanium alloys resulting in less recoil of the frame when expanded which leads to decreased risk of valve embolization, decreased paravalvular leak due to improved conformability of the native anatomy, more accurate restoration of the physiologic EOA, and decrease conduction system injury due to a lower balloon inflation diameter required to obtain the physiologic EOA after balloon inflation, h) has little or no longitudinal foreshortening, which allows for more accurate placement of the valve in the native annulus, since a frame with no longitudinal foreshortening has a shorter initial frame length allowing for a shorter balloon, which decreases conduction system injury, and/or resulting in lower rates of leaflet thrombosis and structural valve deterioration, i) has commissural alignment markers that allows for proper placement of the valve in relation to the native commissures of the valve for proper hemodynamic function in regard to wash out of the valve and blood flow to the coronaries, which leads to better durability and longevity of the valve, and access and re-intervention of the coronaries preventing future adverse events, and/or j) is formed of a metal alloy with no nickel content that prevents allergic response due to the presence of nickel and restenosis associated with nickel content.

Another and/or alternative non-limiting object of the present disclosure is the provision of a medical device for implantation into a heart; the medical device includes an expandable metal frame, a leaflet structure supported by the expandable metal frame; the expandable metal frame is configured to expand from a crimped orientation to an expanded orientation when the medical device is positioned in a treatment site in the heart; the expandable metal frame includes a plurality of angular articulating members and a plurality of axial longitudinal members; and the angular articulating members and the axial longitudinal members are connected together to form a plurality of open cells in the expandable metal frame organized into rows whereby the rows of cells are connected by axial longitudinal members.

Another and/or alternative non-limiting object of the present disclosure is the provision of a medical device wherein a portion or all of the frame of the medical device includes a refractory metal alloy or a metal alloy that includes at least 5 awt. % (e.g., 5-99 awt. % and all values and ranges therebetween) rhenium and/or hafnium.

Another and/or alternative non-limiting object of the present disclosure is the provision of a medical device wherein a portion or all of the outer surface of one or more components (e.g., frame, one or more leaflets, inner skirt, outer skirt, material used to connect leaflets to frame, etc.) of the medical device are coated with an enhancement layer.

Another and/or alternative non-limiting object of the present disclosure is the provision of a medical device wherein a portion or all of the outer surface of one or more components of the medical device are coated with an enhancement layer that includes TiNOx and/or ZrNxOy so as to promote the formation of nitric oxide on the surface of the enhancement layer.

Another and/or alternative non-limiting object of the present disclosure is the provision of a medical device that is coated with an enhancement layer used to a) reduced metal ion release of the metal material from the frame of the medical device, b) reduce the rate of corrosion on the metal that forms the frame of the medical device and/or c) reduces the rate of structural valve disease (SVD) by i) reducing neointimal hyperplasia/cell overgrowth onto one or more portions of the medical device after implantation in the treatment area, ii) reducing infection about the medical device after implantation in the treatment area, iii) reducing platelet activation about the medical device after implantation in the treatment area, iv) reducing thrombosis about the medical device after implantation in the treatment area, v) reducing restenosis about the medical device after implantation in the treatment area, vi) reducing the incidence of nickel from the frame of the medical device reacting with cells about the medical device after implantation in the treatment area, vii) reducing inflammatory cell response about the medical device after implantation in the treatment area, viii) promoting endothelial cell angiogenesis about the medical device after implantation in the treatment area.

Another and/or alternative non-limiting object of the present disclosure is the provision of a medical device that is coated with an enhancement layer (e.g., metal oxynitride layer) that facilitates in the formation of a) nitric oxide (NO) production, b) stimulation of endothelial cells, and/or c) a modulation of endothelial cells.

These and other advantages will become apparent to those skilled in the art upon the reading and following of this description.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 illustrates a typical TAVR procedure for inserting the TAVR valve into a valve of a heart.

FIG. 2 illustrates the deployment of the filter arrangement in the heart prior to the deployment of the TAVR valve.

FIG. 3 illustrates a common heart and the location that the filter arrangement can be deployed in the heart.

FIG. 4 illustrates the deployment of the TAVR valve while the filter arrangement is deployed.

FIG. 5 illustrates the capture of emboli by the filter arrangement when the TAVR valve is deployed at the treatment site.

FIGS. 6-14 illustrate several non-limiting embodiments of filter arrangement that can be used in accordance with the present disclosure.

FIG. 15 illustrates that collapsing of the filter arrangement after the deployment of the TAVR valve at the treatment site.

FIG. 16 illustrates the removal of the delivery system from the treatment site while the filter arrangement is in the collapsed orientation and the deployed TAVR valve remains deployed in the heart.

FIG. 17 illustrates the use of the filter arrangement in accordance with the present disclosure with a medical device that does not include a heart valve such as a balloon angioplasty device.

FIG. 18 is an illustration of a TAVR valve in accordance with the present disclosure.

FIG. 19 is an illustration of the TAVR valve of FIG. 1 illustrating features of the axial longitudinal members and the angular articulating members of the frame.

FIG. 20 is a front elevation view of a frame of a TAVR valve in the expanded state in accordance with the present disclosure.

FIG. 21 is a front view of a flat frame of a TAVR valve in the expanded state in accordance with the present disclosure.

FIG. 22 is a front view of another non-limiting flat frame of a TAVR valve in the expanded state in accordance with the present disclosure.

FIG. 23 is a cross-sectional view of a section of a frame that illustrates an enhancement layer on the outer surface of the section of the frame.

FIG. 24 is a cross-sectional view of a section of a portion of a frame that illustrates an enhancement layer on the outer surface of a pre-applied metal layer that is coated on the outer surface of a section of the frame.

FIG. 25 illustrates various prior art filters that can be used during a TAVR procedure and the also provides some of the features and limitations of such prior art filters.

FIG. 26 is a graph illustrating the incidence of stroke in a patient after a TAVR procedure.

### DESCRIPTION OF NON-LIMITING EMBODIMENTS OF THE DISCLOSURE

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

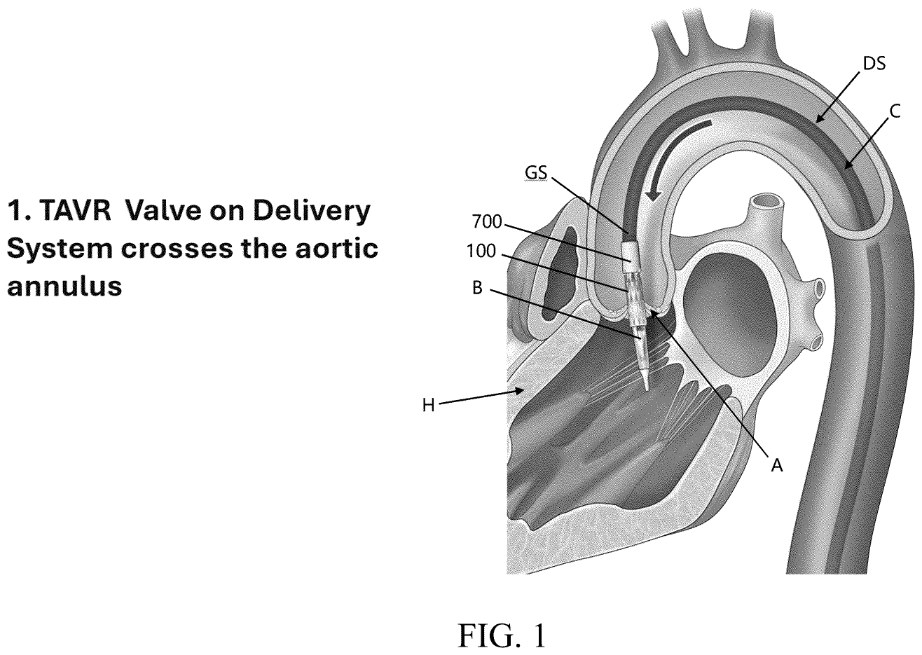

Referring now to FIG. 1, there is illustrated a non-limiting procedure for inserting a prosthetic heart valve 100 into a heart H. The prosthetic heart valve 100 is configured to be radially collapsible to a collapsed or crimped state for introduction into the body on a delivery catheter C and radially expandable to an expanded state for implanting prosthetic heart valve 100 at a desired location in heart H (e.g., aortic valve A, etc.). The frame of prosthetic heart valve 100 is made of a plastically-expandable material that permits crimping of the frame to a smaller profile for delivery and expansion of prosthetic heart valve 100 using an expansion device. FIG. 1 illustrates the prosthetic heart valve 100 crimped on an inflation lumen or balloon B of catheter C. The balloon B on the catheter C can be used to expand the frame of the prosthetic heart valve 100 from a crimped state to an expanded state. Various type of crimping apparatus and techniques can be used to crimp the prosthetic heart valve on the balloon delivery catheter. The process of crimping a prosthetic heart valve using a crimping device is known in the art and will not be described herein. During a crimping procedure, damage to leaflets of leaflet structure should be avoided.

Once prosthetic heart valve 100 is crimped on balloon B of the catheter C, the catheter C is inserted through a blood vessel and to the location in heart H wherein prosthetic heart valve 100 is to be deployed as illustrated in FIG. 1. At the treatment location, the balloon B on catheter C is expanded to thereby cause prosthetic heart valve 100 to be expanded and secured in a valve region A of heart H. Thereafter, balloon B is deflated and the catheter C is removed from the patient.

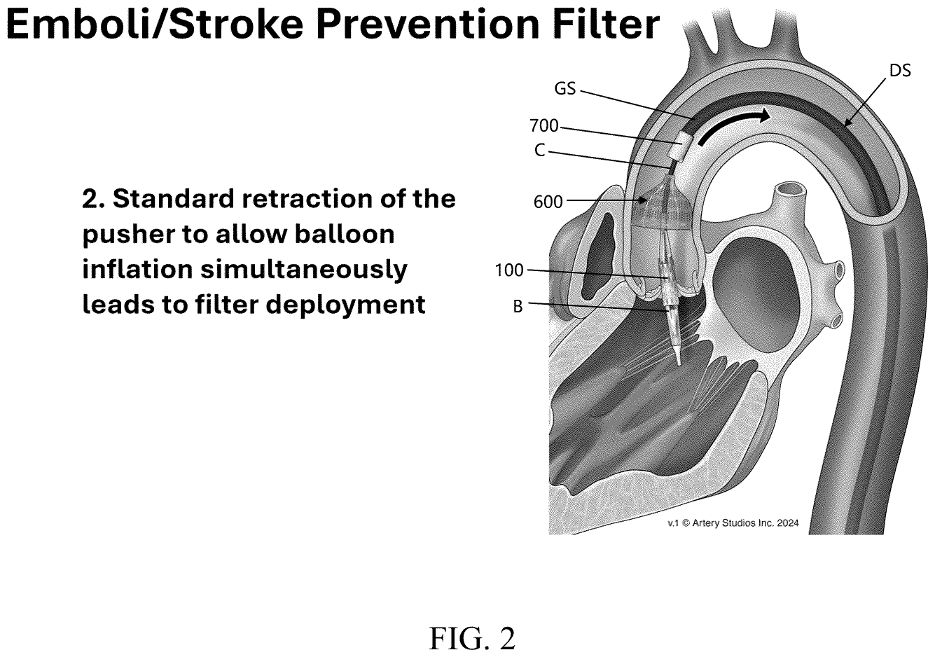

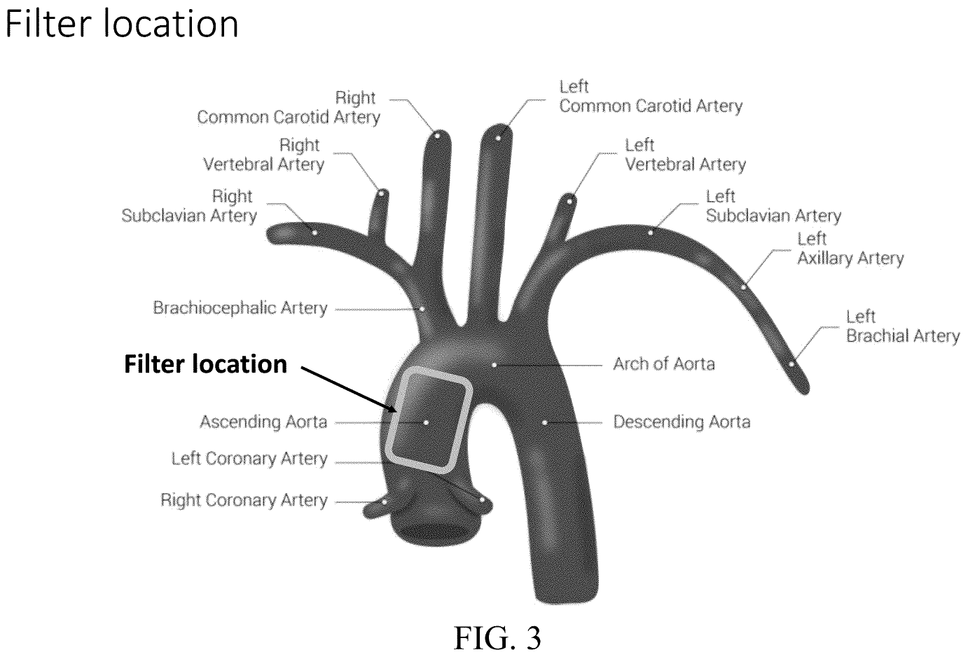

Referring now to FIG. 2, once the prosthetic heart valve 100 is positioned at the treatment area (e.g., aortic annulus), the guide shaft GS is retracted from the filter arrangement 600 to enable the filter arrangement to expand to the deployed or open position. As illustrated in FIG. 2, the front end of the deployed filter arrangement 600 has a cross-sectional area that is as large as or nearly as large as the portion of the aorta where the filter arrangement 600 has deployed. Such a deployment of the filter arrangement 600 results in all or nearly all of the blood moving from the aortic annulus and towards the brachiocephalic artery to pass through the filter material of the filter arrangement 600. As also illustrated in FIG. 2, the deployed filter arrangement 600 is located forwardly of the brachiocephalic artery so as to not interfere with blood flow into the brachiocephalic artery and other arteries located rearwardly of the brachiocephalic artery. FIG. 3 illustrates the general area that the filter arrangement 600 should be deployed in the aorta during a TAVR procedure.

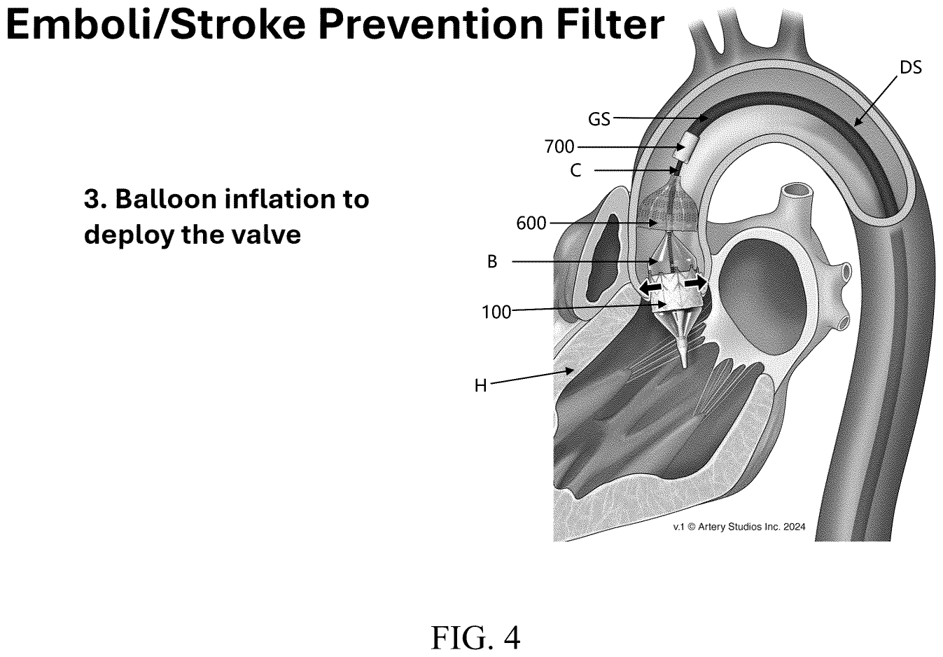

Referring now to FIG. 4, after the filter arrangement 600 is deployed, the prosthetic heart valve 100 can be expanded. When the prosthetic heart valve 100 is a balloon expandable prosthetic heart valve 100, the balloon B on the catheter is expanded to cause the expandable frame of the prosthetic heart valve 100 to expand and engage the inner walls of the aorta.

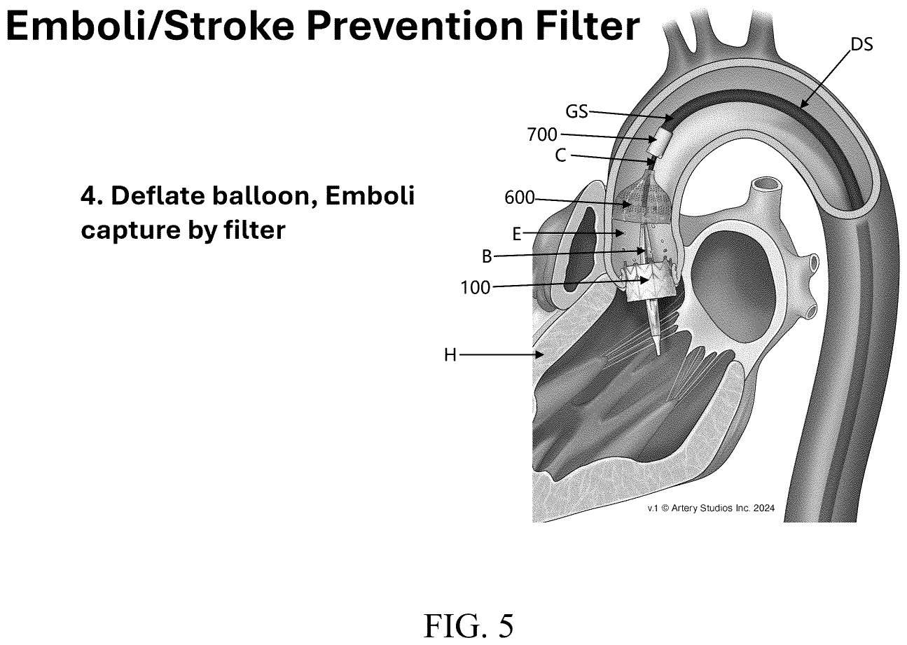

FIG. 5 illustrates the dislodgement of emboli E about the expanded prosthetic heart valve 100 that is captured by the deployed filter arrangement 600 that is located rearwardly to the prosthetic heart valve 100. FIG. 5 also illustrates that the balloon B has been partially or fully deflated after the expansion of the prosthetic heart valve 100. During the expansion of the prosthetic heart valve 100 in the treatment area, emboli E can be caused to be dislodged from the treatment area. Likewise, when the balloon B is deflated, dislodged emboli E from the treatment area may enter the blood stream and flow rearwardly of the expanded prosthetic heart valve 100. The deployed filter arrangement is illustrated as capturing such dislodged emboli E that is contacts the filter material of the deployed filter arrangement and which dislodged emboli E is larger than material size that the filter material is configured to filter.

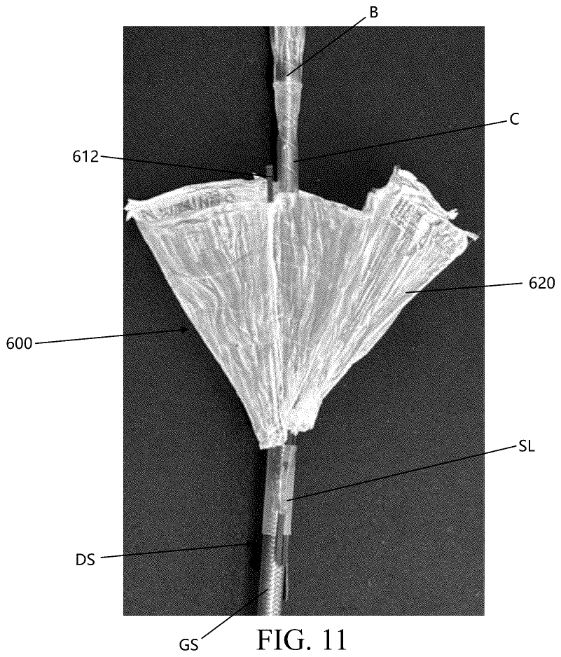

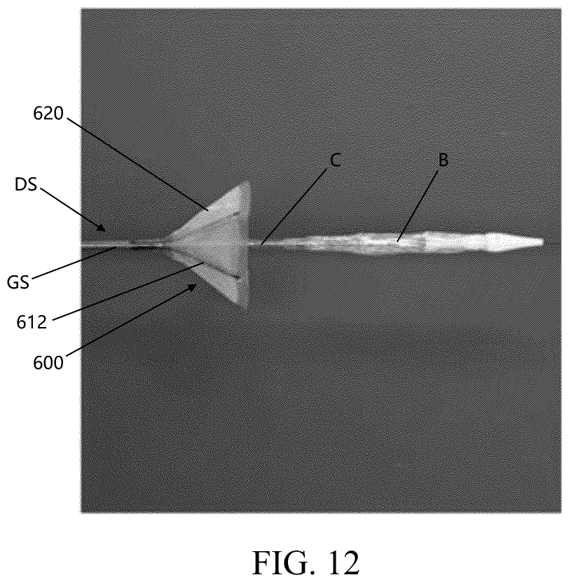

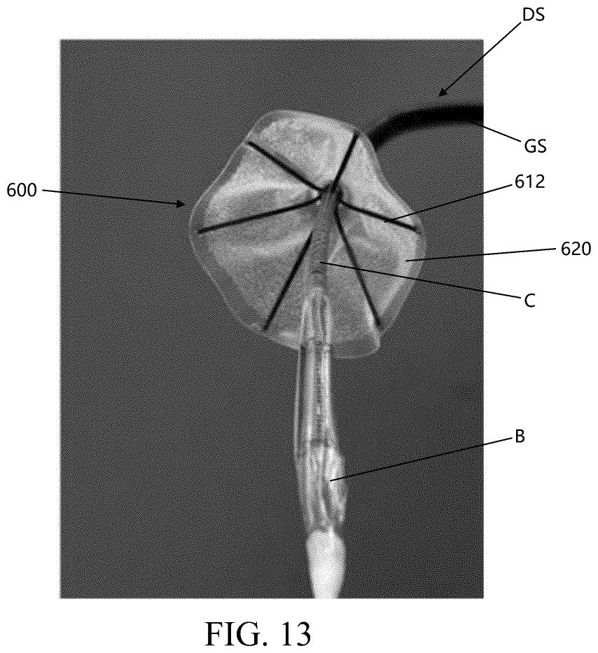

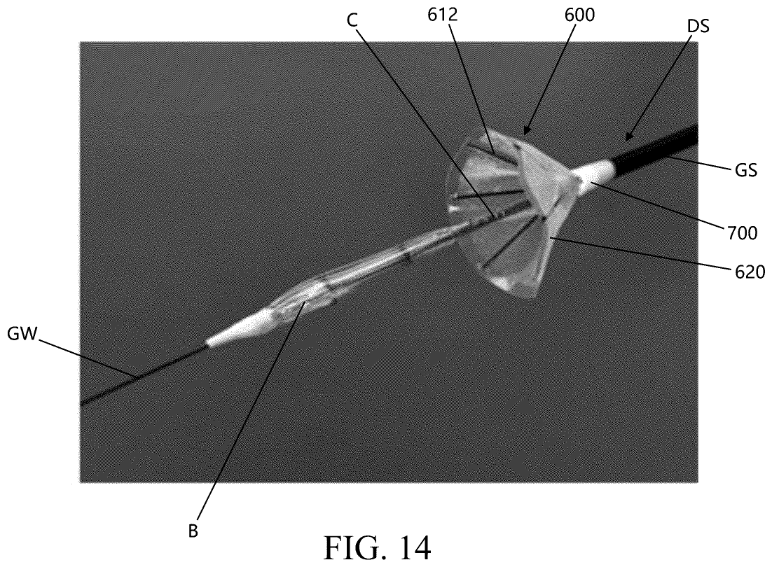

Referring now to FIGS. 4-14, the shape of the deployed filter arrangement 600 is non-limiting. FIGS. 4-14 illustrate several non-limiting embodiments of filter arrangement 600 that can be used in accordance with the present disclosure. The filter frame 610 of the filter arrangement 600 can have many different configurations to cause the filter material 620 to effectively filter dislodged emboli material E from the blood that is larger than material size that the filter material 620 is configured to filter when the prosthetic heart valve 100 is expanded and deployed in the heart H. In one non-limiting embodiment, the filter frame 610 is partially or fully formed of a shape memory material; however, this is not required.

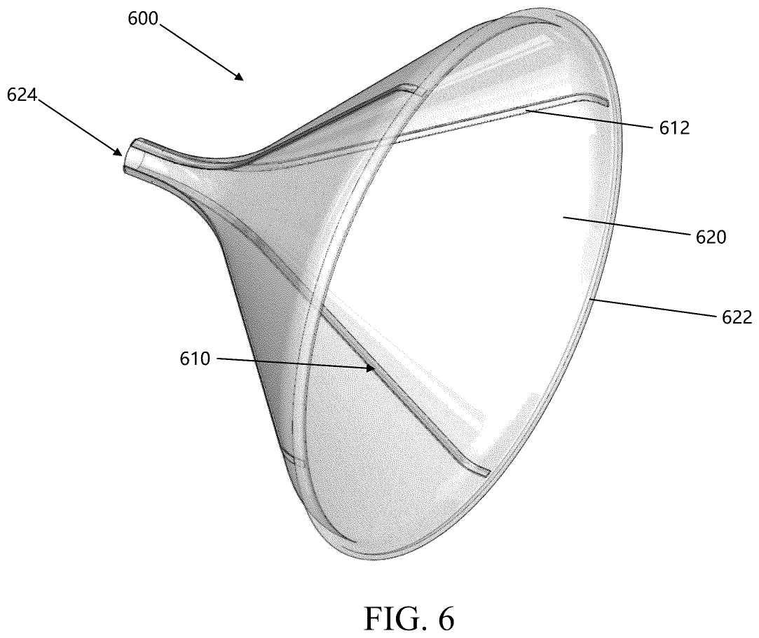

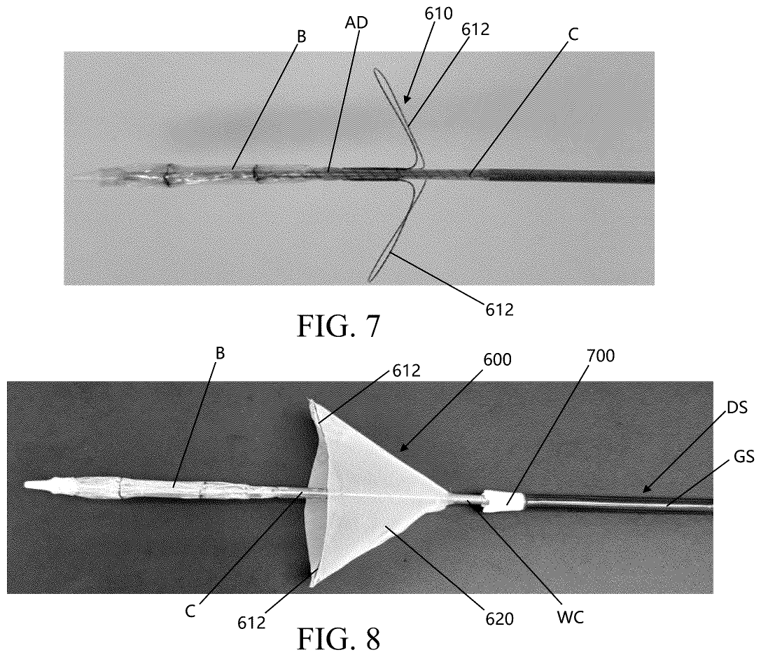

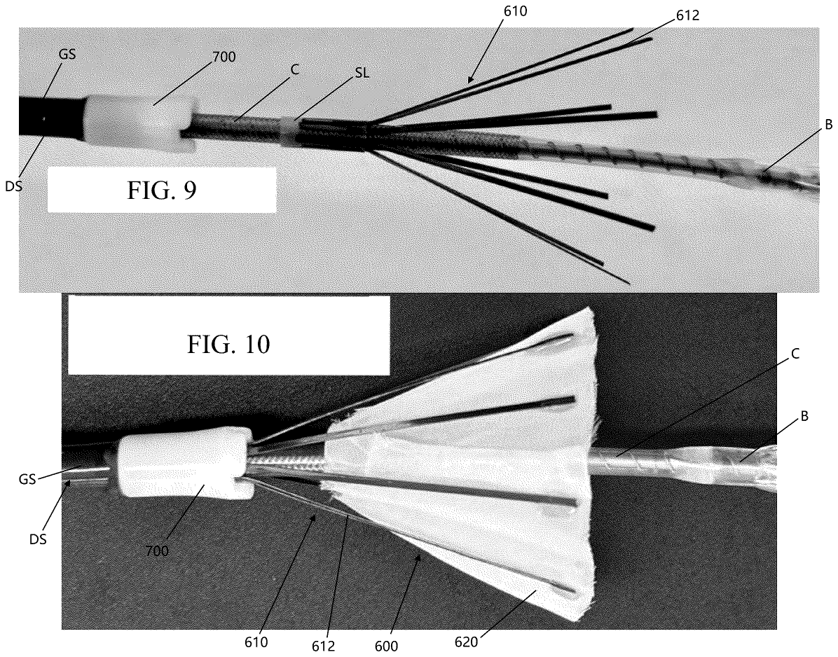

Referring now to FIG. 6, the deployed filter arrangement 600 has a generally conical-shaped or umbrella-shaped profile. The filter frame 610 includes a plurality of frame members 612. The filter frame 610 can be adhered to or otherwise connected to the proximal end of inflation lumen or balloon B; however, proximal end of the filter arrangement 600 is typically connected to the catheter C at some location rearwardly of the inflation lumen or balloon B (e.g., 0.1-20 mm and all values and ranges therebetween rearwardly of the inflation lumen or balloon). The connection arrangement in which the filter frame is connected to the balloon B and/or catheter C is non-limiting. The connection arrangement in which the filter material 620 is connected to the filter frame 610 is non-limiting. For example, as illustrated in FIGS. 9 and 11, a sleeve SL is used to at least partially secure the filter frame 610 to the catheter C. FIGS. 7 and 8 illustrate the use of an adhesive AD and/or a wrapped cord connection WC (e.g., wrapped wire, etc.) used to at least partially secure the filter frame 610 to the catheter C.

The filter material 620 used in the filter arrangement is a biocompatible material. The type of biocompatible material used to form the filter material 620 is non-limiting (e.g., polyurethane, nylon, polyethylene terephthalate, polystyrene, polypropylene, UHM polymers, nylon, nitinol, shape memory material, etc.). The filter material 620 is generally configured to filter out particles from the blood have a particle size that is greater than 450 microns; however, it can be appreciated that the filter material 620 can be configured to filter out particles from the blood have a particle size that are less than 450 microns. For example, the filter material 620 could be configured to filter out particles from the blood having a particle size that is greater than 200 microns. Generally, the filter material 620 is configured to filter out particles from the blood have a particle size that is greater than 50-450 microns (and all values and ranges therebetween); however, other average pore sizes can be used. The thickness of the filter material 620 is non-limiting (e.g., 0.001-0.05 inches and all values and ranges therebetween). The filter material 620 is generally configured to allow at least 0.25 L/min. (0.25-20 L/min. and all values and ranges therebetween) of blood through the filter material 620 when the filter arrangement 600 is deployed. Non-limiting examples of filter material 620 include a) polyurethane film with a thickness in the range of 0.001-0.01 inches (and all values and ranges therebetween) having a plurality of holes, (e.g., laser drilled holes or patterns, etc.), b) an expanded PTFE film (e.g., fully sintered) with a thickness in the range of 0.001-0.01 inches (and all values and ranges therebetween) having a plurality of holes, (e.g., laser drilled holes or patterns, etc.), c) PTFE film (e.g., unsintered) that is used to partially or fully encapsulate the filter frame (e.g., two layers of PTFE film partially or fully encapsulate the frame, and thereafter the two layers are connected together (e.g., sintered together, pressure connected together and heated, etc.), and then a plurality of holes, (e.g., laser drilled holes or patterns, etc.) would be formed in the PTFE film, d) a nylon material (e.g., nylon mesh, lamination of nylon mesh layers, lamination of nylon mesh, etc.), or e) a nylon material laminated to other polymer film (e.g., polyurethane, etc.), wherein the nylon material provides additional strength to the polymer film and the polymer film provides the defined porosity of the laminated material.

The shape of the filter material 620 can optionally be modified to improve the interaction with fluid flow (e.g., ensure the filter material is forced toward the blood vessel wall instead of to the center of the blood vessel when the filter arrangement is deployed). A filter lip arrangement 622 on the distal outermost edge of the filter material 620 can be configured to cause the filter material to deflect outward towards the blood vessel wall when the filter arrangement is deployed blood is flowing through the filter material. As such, the filter lip arrangement 622 on the distal outermost edge (e.g., front end of the filter material 620) of the filter material 620 is configured (e.g., curved inwardly, etc.) to cause the filter material 620 to make contact with or otherwise contact a vasculature wall about the filter arrangement 600 so that most (e.g., 70-99.99% and all values and ranges therebetween) or all of the fluid flows (e.g., blood flow, etc.) through the filter material 600 when the filter arrangement 600 is in said deployed position. One such filter lip arrangement 622 is illustrated in FIG. 6. In one non-limiting arrangement, the filter lip arrangement 622 has a curved profile and extends outwardly from the front end of the filter frame 610. The end of the filter frame has a curved portion that facilitates in the formation of the filter lip arrangement 622; however, other or additional arrangements can be used to form the filter lip arrangement 622. As can be appreciated, other configurations of the filter lip arrangement can be used.

The back end 624 of the filter arrangement 600 generally includes an opening that is configured to encircle a portion of the catheter C and/or other portions of the delivery device DS. The shape of the opening is non-limiting (e.g., circular, oval, polygonal, etc.). Generally, the back end 624 of the filter arrangement 600 is secured to the catheter C and/or other portions of the delivery device DS so as to inhibit or prevent blood and/or dislodged emboli from passing through the opening in the back end 624 of the filter arrangement 600 when the filter arrangement 600 is deployed in the treatment area. As discussed above, the type of connection arrangement used to secure the back end 624 of the filter arrangement 600 to the catheter C and/or other portions of the delivery device DS is non-limiting.

The configuration of the filter frame 610 of the filter arrangement 600 is non-limiting. The filter frame 610 can be configured to form an umbrella-type filter arrangement (See FIGS. 4, 5, 6, 8, 10-14). Such an umbrella-type filter arrangement can include two or more (e.g., 2-14 and all values and ranges therebetween) longitudinal members or legs 612 that are fixed on the proximal end to the inflation lumen or balloon, or at some location rearwardly of the inflation lumen or balloon (e.g., 0.1-20 mm and all values and ranges therebetween rearwardly of the inflation lumen or balloon). The adjacently positioned longitudinal members or legs 612 can optionally be positioned equidistant for one another around the circumference of the inflation lumen, balloon and/or catheter. The longitudinal members or legs 612 can be configured to expand or pivot outwardly a distance (e.g., 15-50 mm and all values and ranges therebetween) to form a cone type shape filter arrangement. The longitudinal members or legs 612 can be formed of a shape memory material (e.g., Nitinol material, shape memory plastic, etc.) that causes the longitudinal members or legs 612 to move to a deployment or open position when not constrained. As can be appreciated, other or additional arrangements can be used to cause the longitudinal members or legs 612 to move to a deployment or open position.

Another non-limiting filter arrangement includes a loop filter frame as illustrated in FIGS. 7 and 8. The loop filter frame includes one or more self-expanding loops 612 (e.g., Nitinol wire loop, shape memory plastic loop, etc.). The size of each loop is about 5-60 mm (and all values and ranges therebetween); however, other sizes can be used. Each of the loops is adhered to or otherwise connected to proximal end of the inflation lumen or balloon, or at some location rearwardly of the inflation lumen or balloon (e.g., 0.1-20 mm and all values and ranges therebetween rearwardly of the inflation lumen or balloon), so that when the filter arrangement is deployed, the expanded end of the deployed filter frame will cause the filter material that is connected to the filter frame to receive blood flow. The loop filter frame forms an inverse clamshell configuration for the filter arrangement. The connection arrangement to which the filter material 620 is connected to the loops 612 is non-limiting. The connection arrangement of the loops 612 to the balloon B and/or catheter C is non-limiting.

One or more structural elements (not shown) formed of material that is more rigid than the filter material (e.g., metal, stiff plastic such as polyimide, etc.) could optionally be placed outside and/or inside of the filter material along a portion of the longitudinal length of the filter arrangement to aid in inhibiting or preventing the filter material from being snagged on the guide shaft (e.g., inhibiting or preventing the filter material from getting snagged on the leading edge of the guide shaft, etc.).

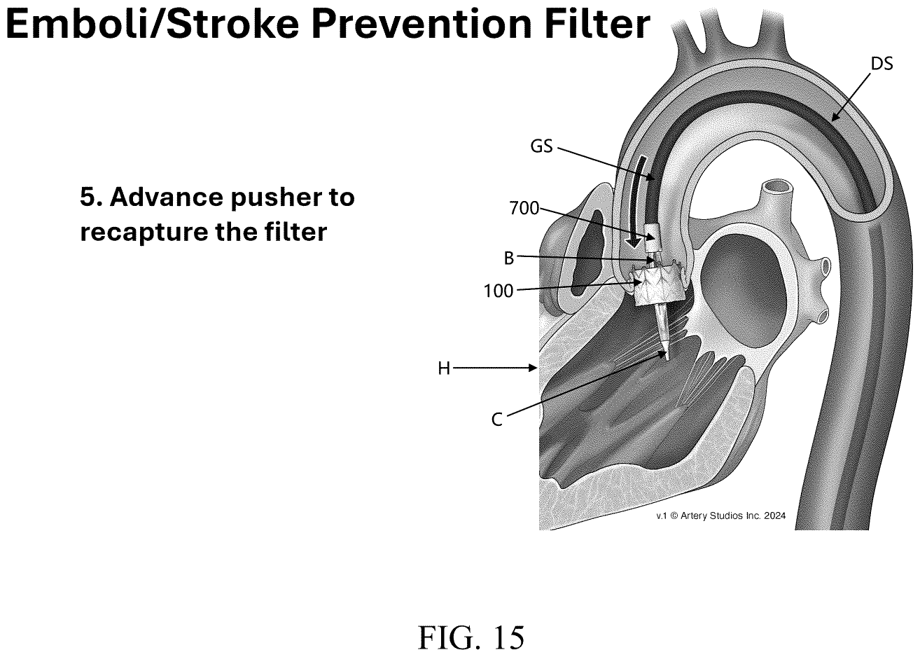

Referring now to FIG. 15, the filter arrangement 600 can be easily and conveniently collapsed and constrained by moving the guide shaft GS forwardly (as illustrated by the arrow) so as to partially or fully cover the filter arrangement 600. As illustrated in FIG. 15, the guide shaft has fully covered the filter arrangement 600; however, this is not required. The front-end portion of the guide shaft GS can optionally include a sleeve 700 that is shaped and sized to facilitate in moving over a portion of all of the filter arrangement 600 during the collapsing of the filter arrangement 600. The front opening of the sleeve 700 can optionally have an opening that has a larger cross-sectional area than the interior passageway of the guide shaft GS. The front opening of the sleeve 700 can optionally have curved and/or angled features used to facilitate in moving the sleeve 700 over a portion of all of the filter arrangement 600 during the collapsing of the filter arrangement 600. The sleeve 700 may optionally be formed of a more durable and/or rigid material than the material used to form the guide shaft GS. During the collapsing of the filter arrangement 600, and emboli material E that was captured by the filter arrangement 600 is retained in the constrained-collapsed or closed position of the filter material 600.

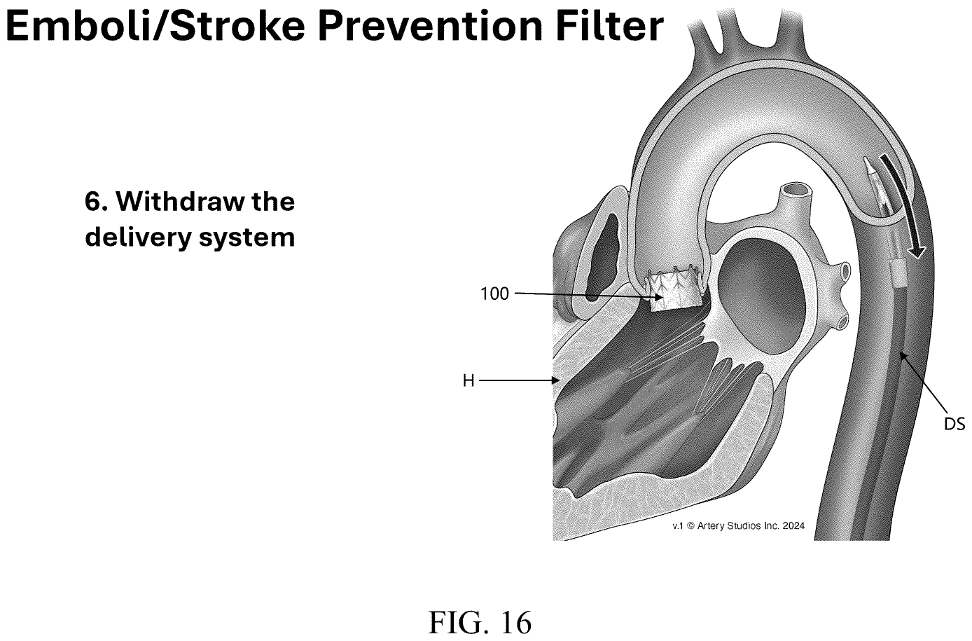

After the filter arrangement 600 has been collapsed and constrained, the delivery device DS can be removed from the patient and the expanded prosthetic heart valve 100 remains in the treatment area as illustrated in FIG. 16. The dislodged emboli material E captured by the filter arrangement 600 is partially or fully encapsulated and trapped in the filter arrangement 600 when the filter arrangement 600 is in the collapsed-constrained or closed position, and such the captured material E is removed from the patient when the delivery device DS is removed from the patient.

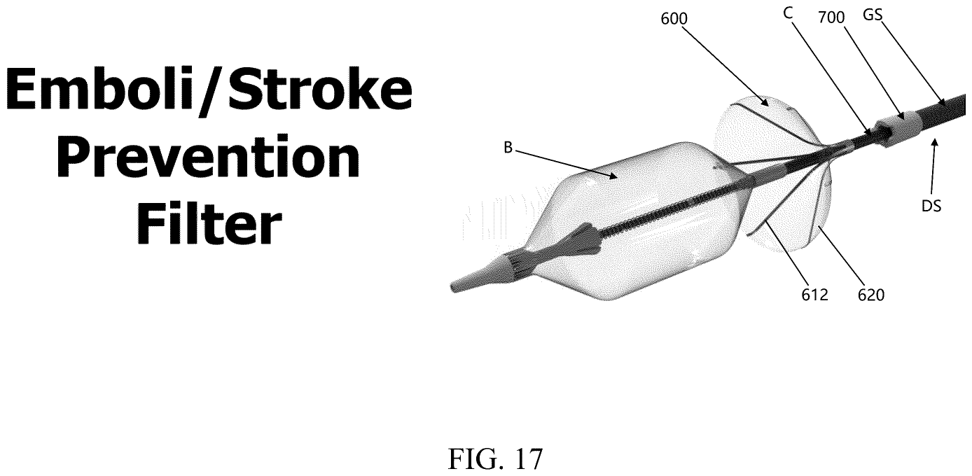

Although the medical device illustrated in FIG. 1 is a TAVR valve, the present disclosure is not limited to TAVR valves or any other heart valve replacement, and includes other expandable devices such as, but not limited to expandable stents, expandable balloons, and other medical devices that includes an expandable frame. For example, referring now to FIG. 17, there is illustrate use of the filter arrangement 600 in accordance with the present disclosure with a medical device that does not include a heart valve such as a balloon angioplasty device B. As can be appreciated, the filter arrangement can be used with an expandable stent.

It is appreciated that when the medical device used with the filter arrangement 600 is a self-expanding medical device (e.g., self-expanding stent, self-expanding prosthetic heart valve, etc.), the catheter may be absent an expandable balloon. In such an arrangement, the filter arrangement 600 is attached or connected to the catheter C at a location that is rearward to the self-expanding medical device.

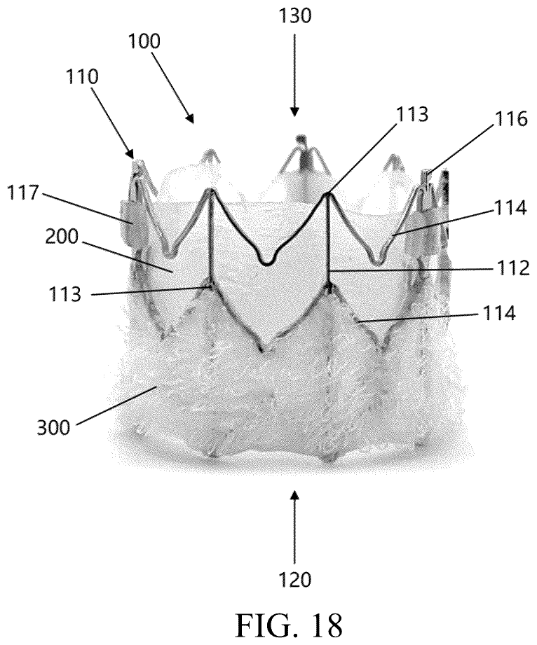

Referring now to FIGS. 18-22, there is illustrated a non-limiting implantable prosthetic heart valve 100 (e.g., TAVR valve, etc.) and a non-limiting frame 110 of an implantable prosthetic heart valve 100. The prosthetic heart valve 100 generally comprises a frame 110 formed of a plurality of axial longitudinal members and angular articulating members or struts 112, 114, strut joints 113, leaflet structure 200 supported by frame 110, and an outer skirt 300 secured to the outer surface of frame 110 and/or leaflet structure 200. The frame can include one or more an orientation structures or commissural markers 116. Prosthetic heart valve 100 has a “lower” end 120 and an “upper” end 130, wherein lower end 120 of prosthetic heart valve 100 is the inflow end and the upper end 130 of prosthetic heart valve 100 is the outflow end.

The configuration of the frame 110 of the prosthetic heart valve 100 is non-limiting. Many different frame configurations can be used for the frame 110 of the prosthetic heart valve 100.

As illustrated in FIGS. 1-2, the frame 110 has a plurality of vertically extending axial longitudinal members 112. The vertically extending axial longitudinal members 112 extend from the top to the bottom of the frame. Non-vertically angular articulating members 114 are connected to the axial longitudinal members 112 at strut joints 113. One or more of the axial longitudinal members and/or angular articulating members 112, 114 can have the same or different a) thicknesses, b) cross-sectional shape, and/or c) cross-sectional area along a portion or all of the longitudinal length.

The frame 110 is partially or fully formed of a metal material. Non-limiting metal materials include a) stainless steel, b) CoCr alloy or MP35N alloy or a Phynox alloy or Elgiloy alloy or L605 alloy, c) TiAlV alloy, d) aluminum alloy, e) nickel alloy, f) titanium alloy, g) tungsten alloy, h) molybdenum alloy, i) copper alloy, j) beryllium-copper alloy, k) Nitinol alloy, 1) refractory metal alloy, or m) metal alloy that includes at least 5 atomic weight percent (awt. %) or atomic percent (awt %) rhenium and/or hafnium (e.g., 5-99 awt. % rhenium and/or hafnium and all values and ranges therebetween). In one non-limiting configuration, 10-100 wt. % of the frame includes refractory metal alloy, or a metal alloy that includes at least 15 atomic weight percent (awt. %) rhenium and/or hafnium.

The outer skirt 300 can be formed of a variety of flexible materials (e.g., polymer (e.g., polyethylene terephthalate (PET), polyester, nylon, Kevlar, silicon, etc.), composite material, metal, fabric material, etc. In one non-limiting embodiment, the material used to partially or fully form outer skirt 300 can be substantially non-elastic (i.e., substantially non-stretchable and non-compressible). In another non-limiting embodiment, the material used to partially or fully form outer skirt 300 can be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). Outer skirt 300 can optionally be formed from a combination of a cloth or fabric material that is coated with a flexible material or with a stretchable and/or compressible material so as to provide additional structural integrity to outer skirt 300. The size, configuration, and thickness of outer skirt 300 is non-limiting (e.g., thickness of 0.1-20 mils and all values and ranges therebetween). The outer skirt 300 can be secured to the outside of the frame 110 using various means (e.g., sutures, clips, clamp arrangement, etc.).

Prosthetic heart valve 100 can optionally include an inner skirt or sleeve (not shown) that is positioned at least partially about the interior region of frame 110. The inner skirt (when used) generally is positioned completely around a portion of the interior of frame 110. Generally, the inner skirt is positioned about the lower portion of frame 110 and does not fully cover the upper portion of frame 110; however, this is not required. The inner skirt can be connected to frame 110 by a variety of arrangements (e.g., sutures, adhesive, melted connection, clamping arrangement, etc.). Generally, the inner skirt is formed of a less flexible and/or compressible material than outer skirt 300; however, this is not required. The inner skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt can optionally be formed from a combination of a cloth or fabric material that is coated with the stretchable and/or compressible material so as to provide additional structural integrity to the inner skirt. The size, configuration, and thickness of the inner skirt is non-limiting. The thickness of the inner skirt is generally 0.1-20 mils (and all values and ranges therebetween).

Outer skirt 300 and/or inner skirt can be used to 1) at least partially seal and/or prevent perivalvular leakage, 2) at least partially secure leaflet structure 200 to frame 110, 3) at least partially protect one or more of the leaflets of leaflet structure 200 from damage during the crimping process of prosthetic heart valve 100, 4) at least partially protect one or more of the leaflets of leaflet structure 200 form damage during the operation of prosthetic heart valve 100 in heart H.

Leaflet structure 200 can be attached to frame 110, inner skirt and/or outer skirt 300. The connection arrangement used to secure leaflet structure 200 to frame 110, inner skirt and/or inner skirt 300 is non-limiting (e.g., sutures, melted bold, adhesive, clamp arrangement, etc.). The material used to form the one or more leaflets of leaflet structure 200 include, but are not limited to, polymers, bovine pericardial tissue, bovine tissue, porcine tissue, biocompatible synthetic materials, or various other suitable natural or synthetic materials.

Leaflet structure 200 can be comprised of two or more leaflets (e.g., 2, 3, 4, 5, 6, etc.). In one non-limiting arrangement, leaflet structure 200 includes three leaflets that are arranged to collapse in a tricuspid arrangement. The size, shape and configuration of the one or more leaflets of leaflet structure 200 are non-limiting. In one non-limiting arrangement, the leaflets generally have the same shape, size, configuration and thickness.

Two of more of the leaflets of leaflet structure 200 can optionally be secured to one another at their adjacent sides to form commissures of leaflet structure 200 (the edges where the leaflets come together). Leaflet structure 200 can be secured to frame 110, inner skirt and/or outer skirt 300 by a variety of connection arrangement (e.g., sutures, adhesive, melted bond, clamping arrangement, etc.).

One or more leaflets of the leaflet structure 200 can optionally include reinforcing structures or strips (not shown) to 1) facilitate in securing the leaflets together, 2) facilitate in securing the leaflets to the inner skirt 300 and/or frame 110, and/or 3) inhibit or prevent tearing or other types of damage to the leaflets.

Prosthetic heart valve 100 is configured to be radially collapsible to a collapsed or crimped state for introduction into the body on a delivery catheter, and radially expandable to an expanded state for implanting prosthetic heart valve 100 at a desired location in heart H (e.g., aortic valve A, etc.). The frame of prosthetic heart valve 100 is made of a plastically-expandable material (e.g., metal alloy, etc.) that permits crimping of the frame to a smaller profile for delivery and expansion of prosthetic heart valve 100 using an expansion device. Various type of crimping apparatus and techniques can be used to crimp the prosthetic heart valve on the balloon delivery catheter.

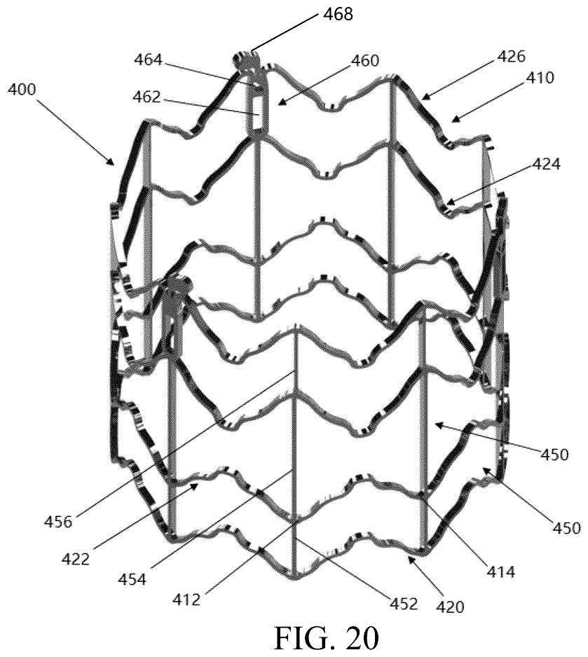

Referring now to FIGS. 20-22, other non-limiting configurations of frame 400 for prosthetic heart valve 100 are illustrated. Frame 400 is configured to be crimped onto a delivery catheter C so that crimped prosthetic heart valve 100 can be inserted in a heart valve. Frame 400 can optionally be configured to enable prosthetic heart valve 100 to be crimped to a diameter that is less than 22 Fr; however, this is not required. As such, prosthetic heart valve 100 that includes frame 400 in accordance with the present disclosure can optionally be configured to enable a prosthetic heart valve 100 to be inserted into smaller sized heart valves that could not previously be treated with prior art prosthetic heart valves. As can be appreciated, prosthetic heart valve 100 in accordance with the present disclosure can be sized and configured to be inserted in heart valves that are larger than 22 Fr.

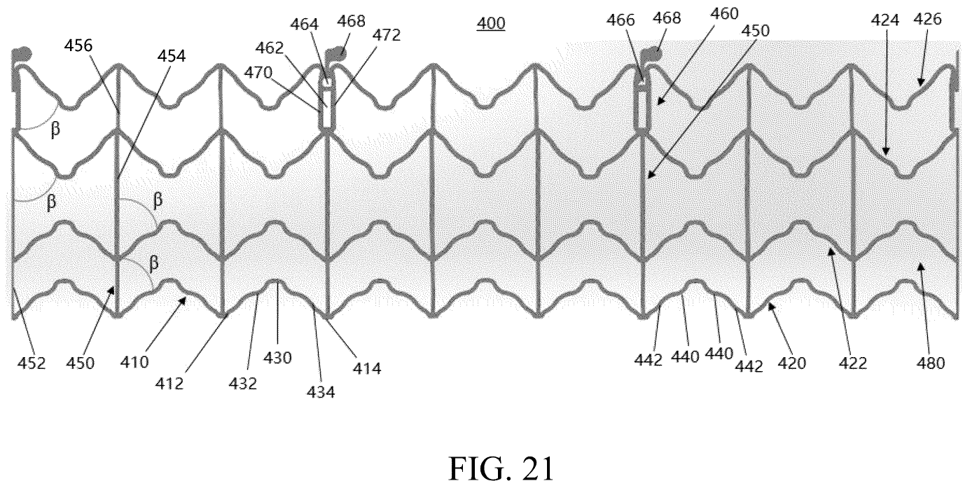

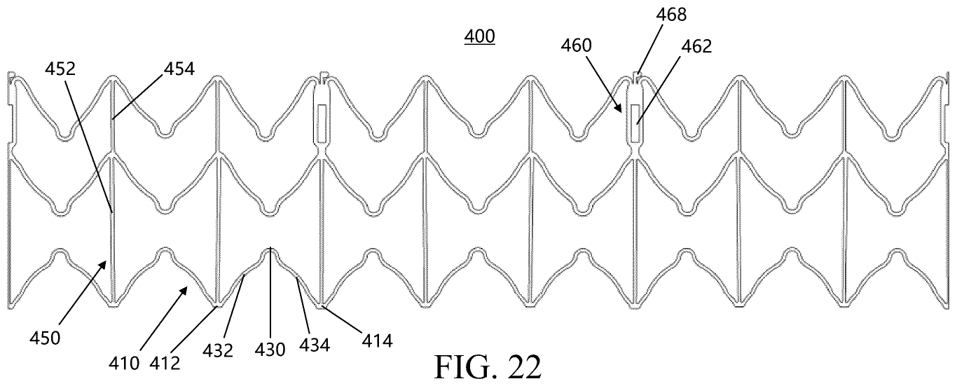

As illustrated in FIGS. 20-21, frame 400 includes four rows of angular articulating members 410 and sets of cells that include nine cells 480, and frame 400 as illustrated in FIG. 22 includes three rows of angular articulating members 410 and sets of cells that include six cells 480.

The radially collapsible and expandable frame 400 includes plurality of angular articulating members 410, a plurality of axial longitudinal members 450, and a plurality of frame opening arrangements 460, and wherein angular articulating members 410, the plurality of axial longitudinal members 450, and frame opening arrangements 460 are connected together to form a plurality of open cells 480 in frame 400. The frame opening arrangements 460 are a type of axial longitudinal member that includes a lower frame opening 462 (e.g., commissure attachment opening for the leaflets) and first and second frame opening struts 470, 472. The central longitudinal axis of frame opening arrangements 460 aligns with the central longitudinal axis of axial longitudinal member segments 452, 454 that are located below frame opening arrangements 460. Likewise, the central longitudinal axis of axial longitudinal member segments 452, 454, 456 are aligned with one another. An open cell is defined as a cell wherein all of the walls of the cell are not formed of angular articulating members 410 or struts. As illustrated in FIGS. 20-22, each of the cells 480 are formed of two angular articulating members 410 and two axial longitudinal members 450, thereby forming an open cell 480.

The region of the frame 400 that includes the frame opening arrangements 460 is referred to as the commissural attachment area. Connected to the top region of the commissural attachment area can optionally include a top marker or orientation structure or commissural alignment marker 468.

The angular articulating members 410 have first and second ends 412, 414 that are connected to axial longitudinal members 450 or frame opening arrangements 460.

Frame opening arrangements 460 are located on the top portion of frame 400. Each of frame opening arrangements 460 can include a lower frame opening 462 and an optional an upper frame opening 464, 466. As illustrated in FIGS. 20 and 21, frame 400 is formed of three sets of cells, wherein each set of cells includes nine cells 480. As illustrated in FIG. 22, frame 400 includes two sets of cells, and wherein each set of cells includes nine cells 480. As illustrated in FIGS. 20-22, the number, shape, and size of cells 480 in each of the rows of cells are mirror images of one another, and have the same shape and size. As illustrated in FIGS. 20-22, the size of the cells in each of the rows can be different and the angular articulating members 410 can be facing in different directions.

Referring again to FIGS. 20 and 21, a plurality of axial longitudinal members 450 are formed of a three axial longitudinal member segments 452, 454, 456, and some of axial longitudinal members 450 are formed of two axial longitudinal member segments 452, 454 and a frame opening arrangements 460. Frame 400 illustrated in FIG. 22 includes a plurality of axial longitudinal members wherein some of the axial longitudinal members are formed of two axial longitudinal member segments and some of the axial longitudinal members are formed of one axial longitudinal member segment and one frame opening arrangement. Axial longitudinal members 450 can be formed of a single piece of material or be formed of a plurality of pieces of material that have been connected together (e.g., adhesive, solder connection, weld connection, band connection, wrapped cord connection, clamp, connection sleeve, etc.). The axial longitudinal member segments that form each of axial longitudinal members 450 are generally aligned along the longitudinal axis of axial longitudinal member 450. The thickness or cross-sectional area of each of axial longitudinal members 450 along the longitudinal axis of the axial longitudinal member can be constant or vary. For example, axial longitudinal member segments 452, 454, 456 have a generally constant thickness or cross-sectional area along the longitudinal length of axial longitudinal member segments 452, 454, 456, whereas frame opening arrangements 460 has a variable thickness or cross-sectional area along the longitudinal length of frame opening arrangements 460. Alternatively, the lower axial longitudinal member segments 452 can optionally have a greater thickness or cross-sectional area than the upper axial longitudinal member segment 456. The middle axial longitudinal member segments 454 can optionally have a greater thickness or cross-sectional area than upper axial longitudinal member segments 456. The lower axial longitudinal member segments 452 can optionally have generally the same thickness or cross-sectional area as middle axial longitudinal member segments 454. As can be appreciated, lower axial longitudinal member segments 452 can optionally have a different thickness or cross-sectional area as middle axial longitudinal member segments 454. The cross-sectional shape of each of the axial longitudinal members 450 along the longitudinal length of axial longitudinal member 450 can be constant or vary. The longitudinal length of the axial longitudinal member segments can be the same or different. The lower axial longitudinal member segments 452 can have a longitudinal length that is less than a longitudinal length of either or both of middle axial longitudinal member segments 454 and upper axial longitudinal member segments 456, and the middle axial longitudinal member segments 454 can have a longitudinal length that is greater than either or both lower axial longitudinal member segments 452 and upper axial longitudinal member segments 456. As can be appreciated, the longitudinal lengths of longitudinal members 452, 454 and 456 can be the same. The longitudinal length of longitudinal member 456 is generally the same length as the longitudinal length of frame opening arrangements 46, absent the commissural alignment marker 468.

As illustrated in FIG. 21, lower axial longitudinal member segments 452 has the shortest longitudinal length, and the middle axial longitudinal member segments 454 has the longest longitudinal length, and the upper axial longitudinal member segment 456 is longer than the lower axial longitudinal member segments 452.

As illustrated in FIGS. 20 and 21, frame 400 includes a first row 420 of angular articulating members 410, a second row 422 of angular articulating members 410, a third row 424 of angular articulating members 410, and a fourth row 426 of angular articulating members 410. First row 420 of angular articulating members 410 is the bottom row and fourth row 426 of angular articulating members 410 is the top row. The shape, size, and/or configuration of angular articulating members 410 of first row 420 are the same. The shape, size, and/or configuration of angular articulating members 410 on second row 422 are the same. The shape, size, and configuration of angular articulating members 410 of third row 424 are the same. The shape, size, and/or configuration of a plurality of angular articulating members 410 on fourth row 426 are the same and a plurality of angular articulating members 410 on fourth row 436 are different. Referring again to FIG. 21, angular articulating members 410 on fourth row 426, wherein either first end 412 or second end 414 the angular articulating members 410 is connected to frame opening arrangements 460, have a different shape, size, and/or configuration from angular articulating members 410 on fourth row 426 wherein both first end 412 and second end 414 of angular articulating members 410 are connected to axial longitudinal members 450. The maximum height of the angular articulating members 410 that are connected to frame opening arrangements 460 are generally the same or less than the maximum height at the top end of upper axial longitudinal member segments 456.

Referring again to FIGS. 20-22, each of the angular articulating members 410 are formed of a centrally located arcuate portion or semi-circular portion 430, and first and second arms 432, 434 that extend from each side of semi-circular portion 430. First arm 432 terminates at first end 412 and second arm 434 terminates at second end 414. Each of first and second arms 432, 434 include one or more undulations 440, 442. As illustrated in FIG. 21, first arm 432 includes first and second undulations 440, 442, wherein the first undulation 440 is located closer to semicircular portion 430 than the second undulation 442. Also, second arm 434 includes first and second undulations 440, 442, wherein first undulation 440 is located closer to semicircular portion 430 than second undulation 442. As such, each angular articulating members 410 includes at least three undulations along a longitudinal length of the angular articulating members 410. As illustrated in FIG. 21, each angular articulating members 410 includes five undulations along the longitudinal length of the angular articulating members 410. As can be appreciated, the angular articulating members 410 can have other shapes (e.g., V-shape, S-shape, Z-shape, W-shape, semi-circular shape, etc.).

As best illustrated in FIG. 21, each of first and second arms 432, 434 of all of angular articulating members 410 include two undulations; however, the shape and size of the undulations for two or more of the rows of angular articulating members 410 is different; however, this is not required. As also illustrated in FIG. 21, the shape and size of the undulations and the location of the undulations on angular articulating members 410 on each row of angular articulating members 410 are generally the same. As illustrated in FIG. 21, the shape and size of the undulations and the location of the undulations the angular articulating members 410 on first and second rows 420, 422 are the same or very similar (e.g., dimensions are less than 5% different). As also illustrated in FIG. 21, the shape and size of the undulations on angular articulating members 410 on the third row are different from first, second and fourth rows 420, 422, 426. Further, the shape and size of the undulations on angular articulating members 410 on the fourth row are different from first, second and third rows 420, 422, 424. In another non-limiting embodiment, for a plurality of angular articulating members 410, the length, shape and/or size of first and second arms 432, 434 are the same or very similar (e.g., dimensions are less than 5% different). In one non-limiting configuration, angular articulating members 410 that form first row 420 of angular articulating members 410 have first and second arms 432, 434 wherein the length, shape, and size of first and second arms 432, 434 are the same. In another non-limiting configuration, angular articulating members 410 that form second row 422 of angular articulating members 410 have first and second arms 432, 434 wherein the length, shape, and size the first and second arms 432, 434 are the same. In another non-limiting configuration, the angular articulating members 410 that form third row 424 of angular articulating members 410 have first and second arms 432, 434 wherein the length, shape, and size of first and second arms 432, 434 are the same. In another non-limiting configuration, angular articulating members 410 that form fourth row 424 of angular articulating members 410 have first and second arms 432, 434 wherein the length and shape of first and second arms 432, 434 are not all the same. In another non-limiting configuration, angular articulating members 410 for first and second rows 420, 422 have first and second arms 432, 434 wherein the length, shape, and size of first and second arms 432, 434 are 410 of first and second arms 432, 434 are the same or very similar (e.g., dimensions are less than 5% different) for angular articulating members 410 for first and second rows 420, 422. In another non-limiting configuration, angular articulating members 410 on each of first, second, third and fourth rows 420, 422, 424 and 426 a) have the same width, and/or b) the center point of semi-circular portion 430 is located with ±5% (and all values and ranges therebetween) the midpoint between adjacently positioned axial longitudinal members 450.

Referring again to FIGS. 20-22, the spacing of angular articulating members 410 between adjacently positioned rows 420, 422, 424, 426 of angular articulating members 410 can be the same or different. In one non-limiting embodiment, the spacing of angular articulating members 410 between adjacent positioned rows (e.g., the first and second rows, the second and third rows, the third and fourth rows, etc.) is different. As illustrated in FIG. 21, the spacing between semi-circular portion 430 of first and second rows 420, 422 of angular articulating members 410 is greater than the spacing between semi-circular portion 430 of second and third rows 422, 424 of angular articulating members 410, and the spacing between first ends 412 of first and second rows 420, 422 of angular articulating members 410 is less than the spacing between first ends 412 of second and third rows 422, 424 of angular articulating members 410, and the spacing between second ends 414 of first and second rows 420, 422 of angular articulating members 410 is less than the spacing between second ends 414 of second and third rows 422, 424 of angular articulating members 410. As also illustrated in FIG. 21, semi-circular portion 430 of first and second rows 420, 422 of angular articulating members 410 are oriented toward the top of the frame, and semi-circular portion 430 of third and fourth rows 424, 425 of angular articulating members 410 are oriented toward the bottom of the frame. As such, the semi-circular portion 430 of second and third rows 422, 424 of angular articulating members 410 face one another. As also illustrated in FIG. 21, the spacing between semi-circular portion 430 of third and fourth rows 424, 426 of angular articulating members 410 is greater than the spacing between semi-circular portion 430 of first and second rows 420, 422 of angular articulating members 410, and the spacing between first ends 412 of third and fourth rows 424, 426 of angular articulating members 410 is greater than the spacing between first ends 412 of first and second rows 420, 422 of angular articulating members 410, and the spacing between second ends 414 of third and fourth rows 424, 426 of angular articulating members 410 is greater than the spacing between second ends 414 of first and second 420, 422 of angular articulating members 410. As also illustrated in FIG. 21, the spacing between semi-circular portion 430 of third and fourth rows 424, 426 of angular articulating members 410 is greater than the spacing between semi-circular portion 430 of second and third rows 422, 424 of angular articulating members 410, and the spacing between first ends 412 of third and fourth rows 424, 426 of angular articulating members 410 is less than the spacing between first ends 412 of second and third rows 422, 424 of angular articulating members 410, and the spacing between second ends 414 of third and fourth rows 424, 426 of angular articulating members 410 is less than the spacing between second ends 414 of second and third rows 422, 424 of angular articulating members 410.

Referring now to FIGS. 20-21, frame opening arrangements 460 are located between third and fourth rows 424, 426 of angular articulating members 410, and FIG. 22 illustrates that the frame opening arrangements 460 are located between second and third rows 424, 426 of angular articulating members 410. As can be appreciated, one or more frame opening arrangements 460 can be located on other regions of frame 400. Frame opening arrangements 460 can optionally be used as securing locations for one of more leaflet structures 200; however, it can be appreciated that one or more of frame opening arrangements 460 can optionally be used as securing locations for other structures (e.g., leaflet, inner skirt, outer skirt, etc.), and/or be used as an indicator of the orientation and/or location of frame 400 in a body passageway or heart valve. Alternatively, an orientation structure 490 can be included in the frame 400.

As illustrated in FIGS. 20-22, each of frame opening arrangements 460 includes first and second frame opening struts 470, 472 that form a lower frame opening 462 and an optional an upper frame opening 464, 466 therebetween. The size and shape of lower frame opening 462 and optional an upper frame opening 464, 466 are non-limiting. As illustrated in FIGS. 20 and 21, lower frame opening 462 has a generally rectangular shape and extends only partially along the longitudinal length of frame opening arrangement 460. As can be appreciated, lower frame opening 462 can have other shapes and sizes. In one non-limiting configuration, each of frame opening arrangements 460 includes a lower frame opening 462 and lower frame openings 462 all have the same or very similar (e.g., dimensions are less than 5% different) shape and size. In one non-limiting embodiment, one or both of first and second frame opening struts 470, 472 a) has a longitudinal axis that is parallel to the longitudinal axis of axial longitudinal member 450 to which the bottom of frame opening arrangements 460, and/or b) has a longitudinal axis that is offset from the longitudinal axis of axial longitudinal member 450 to which the bottom of frame opening arrangements 460. As illustrated in FIGS. 20 and 21, both of first and second frame opening struts 470, 472 a) has a longitudinal axis that is parallel to the longitudinal axis of axial longitudinal member 450 to which the bottom of frame opening arrangements 460, and b) has a longitudinal axis that is offset from the longitudinal axis of axial longitudinal member 450 to which the bottom of frame opening arrangements 460 is connected thereto. The longitudinal length of one or both of first and second frame opening struts 470, 472 can be the same or less than the longitudinal length of length for an axial longitudinal member segment that is located adjacent to first and second frame opening struts 470, 472. As illustrated in FIG. 21, the longitudinal length of first and second frame opening struts 470, 472 is about the same as the longitudinal length of length of axial longitudinal member segment 456.

As illustrated in FIG. 21, the end of first or second arms 432, 434 of angular articulating members 410 of fourth row 426 that is connected to frame opening arrangements 460 can optionally be configured to angle downwardly, and the other end of first or second arms 432, 434 of angular articulating members 410 that is connected to an axial longitudinal member segment is configured to angle upwardly. As illustrated in FIG. 21, the ends of first and second arms 432, 434 of angular articulating members 410 of first, second and third rows 440, 422 and 424 that is connected to an axial longitudinal member segment are both angled in the same direction. As illustrated in FIG. 21, the angle β of angular articulating members 410 relative to axial longitudinal members 450 when the frame is in the expanded orientation is generally 25-60°(and all values and ranges therebetween).

Referring now to FIGS. 20-22, frame opening arrangements 460 can optionally include one or more optional upper frame openings 464, 466. One or more optional upper frame openings 464, 466 are generally positioned above lower frame opening 462. Generally, one or more optional upper frame openings 464, 466 have a cross-sectional area or size that is less than lower frame opening 462; however, this is not required. As illustrated in FIGS. 20 and 21, the shape of two or more of optional upper frame openings 464, 466 are different. The different shapes of one or more optional upper frame openings 464, 466 can be used as a marker to facilitate in the proper positioning of frame 400 and prosthetic heart valve 100 in the heart. In one specific non-limiting configuration, each of one more optional upper frame openings 464, 466 has a different shape. As illustrated in FIG. 20, two of frame opening arrangements 460 include two different shaped upper frame openings 464, 466 and other frame opening arrangements 460 is absent an upper frame opening.

The top portion of each of frame opening arrangements 460 can optionally include a top marker 468. The shape and size of top marker 468 (when used) is non-limiting. As illustrated in FIGS. 20-22, the shape and size of markers 468 are the same or very similar (e.g., dimensions are less than 5% different). Top markers 468 can be used as a marker to facilitate in the proper positioning of frame 400 and prosthetic heart valve 100 in the heart. The one or more top markers 468 (when used) can also or alternatively be used to enable one or more components of prosthetic heart valve 100 (e.g., leaflet, inner skirt, outer skirt, etc.) to be connected to frame 400. The top markers 468 can be formed of the same or different material from other portions of frame 400.

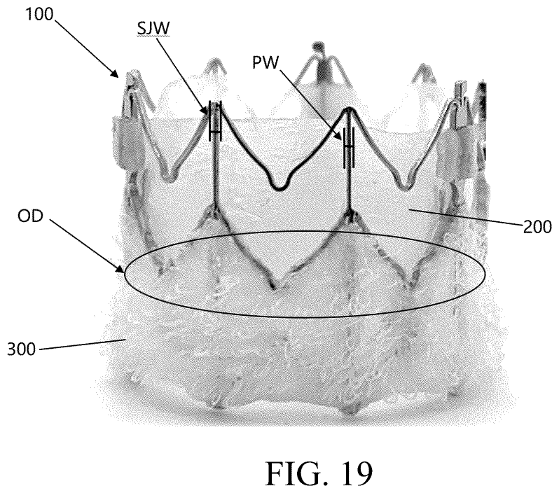

Referring now to FIG. 19, when the frame 110 is formed of a refractory metal alloy or metal alloy that includes at least 5 awt. % (e.g., 5-99 awt. % and all values and ranges therebetween) rhenium and/or hafnium, the post width PW and/or the strut joint width SJW of a frame 110 that is formed of such metal alloy can be smaller than the post width PW and/or the strut joint width SJW of a similar shaped and configured frame formed of stainless steel, nitinol, Co-Cr alloy or TiAlV alloy, and still have the same or greater radial strength when the frame is expanded as compared to a frame formed of stainless steel, nitinol, Co-Cr alloy or TiAlV alloy.

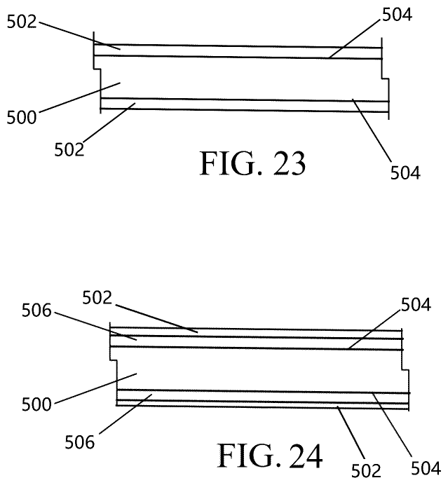

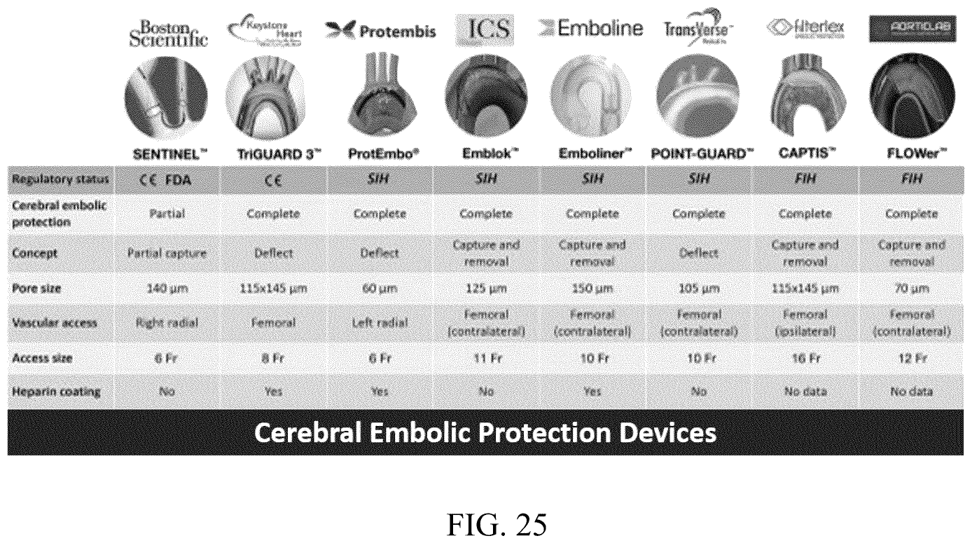

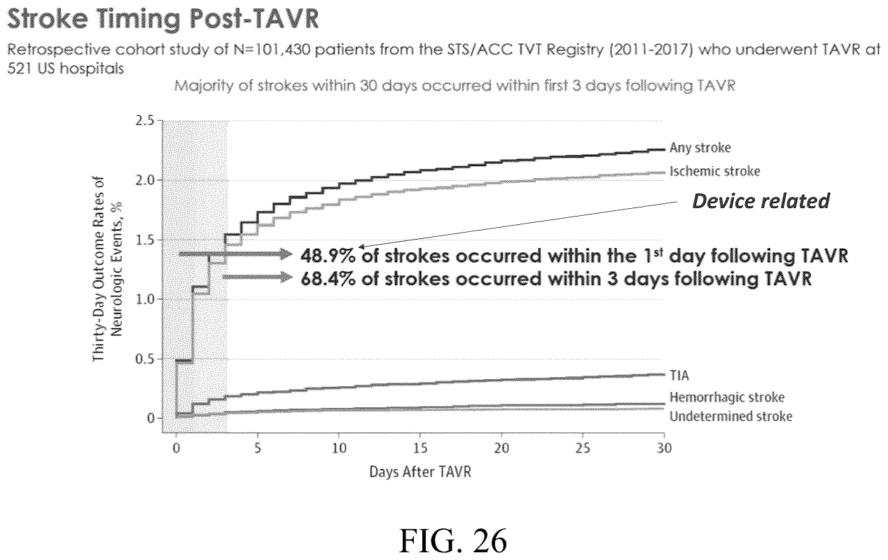

Referring now to FIG. 23, there is illustrated a cross-sectional view of a section of a frame 110, 400 that has an optional enhancement layer 502 on the outer surface 504 of the section of the base material 500 of the frame. Although FIG. 23 only illustrated a coating on the outer surface of frame 110, 400, the enhancement layer 502 can also or alternatively be coated on one or more other components of the prosthetic heart valve 100 such as, but not limited to, the inner skirt, the outer skirt, one or more of all of the leaflets, and/or the material used to secure leaflets to frame. In one non-limiting configuration, 10-100% (and all values and ranges therebetween) of the outer surface 504 of frame 110, 400 is coated with enhancement layer 502. In another non-limiting configuration, the frame and one or more of the inner skirt, the outer skirt, and/or one or more of all of the leaflets are coated with enhancement layer 502, and 10-100% (and all values and ranges therebetween) of the outer surface 504 of frame 110, 400 is coated with enhancement layer 502, and 10-100% (and all values and ranges therebetween) of the outer surface of one or more of the inner skirt 300, the outer skirt, and/or one or more of all of the leaflets 200 are coated with enhancement layer 502. As can be appreciated, when an enhancement layer is not used, layer 502 could be a layer of agent.

Referring now to FIG. 24, the enhancement layer 502 can alternatively be coated on a metal coating layer 506 (e.g., titanium layer, zirconium layer, etc.), which is in turn coated on to the outer surface 504 of the base material 500. The type of metal material used on the optional metal coating layer 506 is non-limiting. The thickness of the metal coating layer 506 is generally at least 0.05 microns, and typically 0.05-15 microns. The coating process to apply the metal coating layer 506 on the outer surface is non-limiting (e.g., PVD, CVD, ALD, PE-CVD in an inert environment, etc.). Although not shown, it can be appreciated that a layer of agent could be coated on the top surface of the enhancement layer. As can be appreciated, layer 506 could be the enhancement layer and layer 502 could be a layer of agent.

The enhancement layer 502 can be used to improve one or more properties of the prosthetic heart valve (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement layer forms a surface that is less of an irritant to cell about the coated surface after the prosthetic heart valve is implanted, reduces the rate to which cells grown on coated surface after the prosthetic heart valve is implanted, reduce rate to which leaflets fail to properly operate after the prosthetic heart valve is implanted, facilitate in nitric oxide generation on the surface of the coating, etc.).

Non-limiting enhancement layers 502 that can be applied to a portion or all of the outer surface of one or more components of the prosthetic heart valve includes chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium-nitrogen-carbon (ZrNC), zirconium OxyCarbide (ZrOC), zirconium oxynitride (ZrNxOy), and combinations of such coatings. In one one-limiting configuration, a portion or all of the outer surface of one or more components of the prosthetic heart valve includes titanium oxynitride or titanium nitride oxide (TiNOx) and/or zirconium oxynitride (ZrNxOy). The enhancement layer 502 can optionally be applied to a portion or all of the outer surface of one or more components of the prosthetic heart valve by a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process.

In one non-limiting embodiment, when forming a titanium oxynitride or titanium nitride oxide (TiNOx) coating on the prosthetic heart valve, the portion of the prosthetic heart valve that is to be coated can be optionally initially coated with Ti metal. The Ti metal coating, when applied, can be applied by PVD, CVD, ALD and PE-CVD in an inert environment. The coating thickness of Ti metal is 0.05-1 microns. Thereafter, the Ti metal coating is exposed to a nitrogen and oxygen mixture that can include nitrogen gas, oxygen gas, a nitrogen containing gas compound and/or an oxygen containing gas compound to cause the nitrogen and oxygen to react with the Ti metal coating. During the formation of the titanium oxynitride or titanium nitride oxide (TiNOx) coating, titanium particles can also be applied to the outer surface of the Ti metal coating prior to and/or during the exposure of the Ti metal coating to the nitrogen and oxygen mixture. The ratio of the N to the O can be varied to control the amount of O in the TiNOx coating. The ratio of N to O when forming the TiNOx coating is generally 1:10 to 10:1 (and all values and ranges therebetween). The coating thickness of the TiNOx coating is generally 0.1-2 microns (and all values and ranges therebetween).

In another non-limiting embodiment, when forming a titanium oxynitride or titanium nitride oxide (TiNOx) coating on the prosthetic heart valve, the portion of the prosthetic heart valve that is to be coated is exposed to titanium particles and a nitrogen and oxygen mixture that can include nitrogen gas, oxygen gas, a nitrogen containing gas compound and/or an oxygen containing gas compound to cause the nitrogen and oxygen to react with the Ti particles. In this coating method, a Ti coating is not preapplied to the outer surface of any portion of the prosthetic heart valve that is to be coated with titanium oxynitride or titanium nitride oxide (TiNOx). The ratio of the N to the O can be varied to control the amount of O in the TiNOx coating. The ratio of N to O when forming the TiNOx coating is generally 1:10 to 10:1 (and all values and ranges therebetween). The coating thickness of the TiNOx coating is generally 0.1-2 microns (and all values and ranges therebetween).

In one non-limiting embodiment, when forming a zirconium oxynitride (ZrNxOy) coating on the prosthetic heart valve, the portion of the prosthetic heart valve that is to be coated can be optionally initially coated with Zr metal. The Zr metal coating, when applied, can be applied by PVD, CVD, ALD and PE-CVD in an inert environment. The coating thickness of Zr metal is 0.05-1 microns. Thereafter, the Zr metal coating is exposed to a nitrogen and oxygen mixture that can include nitrogen gas, oxygen gas, a nitrogen containing gas compound and/or an oxygen containing gas compound to cause the nitrogen and oxygen to react with the Zr metal coating. During the formation of the zirconium oxynitride (ZrNxOy) coating, zirconium particles can also be applied to the outer surface of the Zr metal coating prior to and/or during the exposure of the Zr metal coating to the nitrogen and oxygen mixture. The ratio of the N to the O can be varied to control the amount of O in the ZrNxOy coating. The ratio of N to O when forming the ZrNxOy coating is generally 1:10 to 10:1 (and all values and ranges therebetween). The coating thickness of the ZrNxOy coating is generally 0.1-2 microns (and all values and ranges therebetween).

In another non-limiting embodiment, when forming a zirconium oxynitride (ZrNxOy) coating on the prosthetic heart valve, the portion of the prosthetic heart valve that is to be coated is exposed to zirconium particles and a nitrogen and oxygen mixture that can include nitrogen gas, oxygen gas, a nitrogen containing gas compound and/or an oxygen containing gas compound to cause the nitrogen and oxygen to react with the Zr particles. In this coating method, a Zr coating is not preapplied to the outer surface of any portion of the prosthetic heart valve that is to be coated with zirconium oxynitride (ZrNxOy) coating. The ratio of the N to the O can be varied to control the amount of O in the ZrNxOy coating. The ratio of N to O when forming the ZrNxOy coating is generally 1:10 to 10:1 (and all values and ranges therebetween). The coating thickness of the ZrNxOy coating is generally 0.1-2 microns (and all values and ranges therebetween).

When the frame 110 of the prosthetic heart valve does not include an enhancement layer 502, the prosthetic heart valve can optionally include one or more agents coated one or more regions of the prosthetic heart valve. Also, when the prosthetic heart valve does include an enhancement layer 502, the prosthetic heart valve can optionally include one or more agents coated on the top surface of the enhancement layer 502.

A non-limiting method for the use of the prosthetic heart valve delivery arrangement that includes a prosthetic heart valve, a delivery system that includes a catheter and a guide shaft, and a filter arrangement is as follows:

The filter frame and filter material of the filter arrangement are folded and a guide shaft is advanced partially or fully over the filter arrangement so as to maintain the filter arrangement in a constrained or closed state.

The delivery system with the prosthetic heart valve on the balloon of the inflation lumen or balloon, is advanced through the patient's anatomy while both the prosthetic heart valve and filter arrangement are a constrained closed or crimped state until the prosthetic heart valve is positioned in the desired delivery location in the heart.

The prosthetic heart valve is deployed in the heart after deployment of the filter arrangement. The filter arrangement is deployed before or during the deployment of the prosthetic heart valve. The filter arrangement can be deployed by retracting the guide shaft from the filter arrangement. The prosthetic heat valve can be deployed by inflating the inflation balloon on the catheter.

After the prosthetic heart valve is deployed in the heart, the filter arrangement is again constrained by advancing the guide shaft over a portion or all of the filter arrangement. If an inflation ballon is used, the inflation balloon can be deflated prior to, during and/or after the constrainment of the filter arrangement.

Remove the delivery system and filter arrangement from the patient while the deployed prosthetic heart valve remains at the treatment site.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

1. A vascular delivery device arrangement comprising a delivery system and a filter arrangement; said delivery system includes a catheter and a guide shaft; said catheter and said guide shaft are configured to move independently from one another; at least a portion of said catheter is telescopically received in a cavity of said guide shaft; said filter arrangement is connected to a distal region of said catheter such that at least a portion or all of the filter arrangement is located proximal to an expandable medical device that is releasably positioned on said catheter; said filter arrangement is configured to move between a constrained-collapsed position and a deployed position; said filter arrangement includes a filter frame and a filter material; said filter material is connected to said filter frame; said filter frame is connected to said catheter; said filter frame is formed or a) a plurality of arms that are spaced from another, or b) a plurality of loops that are spaced from one another; said guide shaft is moveable along a longitudinal axis of said catheter and relative to said filter arrangement between a constraining and non-constraining position; said filter arrangement is positioned in said constrained-collapsed position when said guide shaft is in said constraining position due to said guide shaft being positioned partially or fully over said filter arrangement; said filter arrangement is moveable to said deployed position when said guide shaft is in said non-constraining position due to said guide shaft being positioned partially or fully proximal to said filter arrangement.

2. The device delivery arrangement as defined in claim 1, wherein said catheter includes a balloon catheter and wherein the expandable medical device can be positioned at least partially over a balloon on said catheter.

3. The device delivery arrangement as defined in claim 1, wherein said filter frame includes shape memory material.

4. The device delivery arrangement as defined in claim 1, wherein a portion of said filter frame of said filter arrangement that is connected to said catheter defines the proximal end of said filter arrangement.

5. The device delivery arrangement as defined in claim 1, wherein said filter material is configured to filter particles having a size of 450 microns and greater.

6. The device delivery arrangement as defined in claim 1, wherein said filter material is configured to allow at least 0.25 L/min. of fluid flow through said filter material.

7. The device delivery arrangement as defined in claim 1, wherein said filter material is at least partially polymeric and/or metallic.

8. The vascular delivery device arrangement as defined in claim 1, wherein said filter material is at least partially formed of polyurethane, polyethylene terephthalate, polystyrene, polypropylene, UHM (Ultra-high-molecular-weight) polyethylene polymers, nylon, nitinol, shape memory material and/or metallic film.

9. The vascular delivery device arrangement as defined in claim 1, wherein said filter arrangement is configured to cause said filter material to contact a vasculature wall when said filter arrangement in said deployed position so that at least a portion of fluid flows through said filter material when said filter arrangement is in said deployed position.

10. The vascular delivery device arrangement as defined in claim 1, wherein said filter frame forms an umbrella-shaped filter arrangement, conical-shaped filter arrangement, or a clamshell-shaped filter arrangement.

11. The vascular delivery device arrangement as defined in claim 1, wherein said filter frame includes at least three longitudinal members that each have first and second ends, said first end of each of said longitudinal members are spaced from one another and are each connected to said catheter.

12. The vascular delivery device arrangement as defined in claim 1, wherein said filter frame contains at least two self-expanding loops, said filter material is connected said catheter at a location that is proximal to a location that each of said self-expanding loops are connected to said catheter; each of said self-expanding loops are spaced from one another.

13. The vascular delivery device arrangement as defined in claim 1, wherein said vascular delivery device arrangement is configured to deliver the expandable medical device to a treatment site; the expandable medical device is prosthetic valve.

14. A medical device delivery arrangement comprising said expandable medical device and said vascular delivery device arrangement as defined in claim 1, wherein said expandable medical device is a prosthetic heart valve that has an expandable frame.

15. The medical vascular delivery device arrangement as defined in claim 14, wherein said catheter includes a balloon and said expandable medical device is positioned at least partially over said balloon on said catheter.

16. The medical vascular delivery device arrangement as defined in claim 14, wherein said expandable frame is at least partially formed of a) stainless steel, b) cobalt-chromium alloy, c) titanium-aluminum-vanadium alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, m) metal alloy that includes at least 15 atomic weight percent (awt. %) rhenium and/or hafnium, or n) a shape memory material.

17. The medical vascular delivery device arrangement as defined in claim 14, wherein, said expandable frame is at least partially formed of a rhenium and/or hafnium containing alloy.

18. The medical vascular delivery device arrangement as defined in claim 14, wherein said expandable frame includes no more than 0.1 wt. % nickel, no more than 0.1 wt. % chromium, and/or no more than 0.1 wt. % cobalt.

19. The medical vascular delivery device arrangement as defined in claim 14, wherein said catheter is a coaxial catheter and said expandable medical device and said filter arrangement are positioned at least partially on or within said coaxial catheter when said medical device is in an unexpanded position.

20. The medical vascular delivery device arrangement as defined in claim 14, wherein an outer surface of at least one component of said expandable medical device includes an enhancement material that has been layered or coated on said outer surface; said enhancement material is formulated to i) provide nitric oxide, and/or ii) promote generation of nitric oxide; said enhancement material is at least partially formulated of oxynitride.

21. The medical vascular delivery device arrangement as defined in claim 14, wherein said expandable medical device includes one or more leaflets, and one or more of an inner skirt, an outer skirt, and/or sutures.

22. A method for deploying a medical device at a treatment site in a vascular system or other body passageway comprising:
a. providing a medical device; said medical device is consisting prosthetic valve that has an expandable frame;
b. providing a device delivery arrangement as defined in claim 1, wherein said device delivery arrangement is configured to deliver said medical device to said treatment site;
c. folding said filter arrangement to said constrained-collapsed position;
d. moving said guide shaft over at least a portion of said filter arrangement to maintain said filter arrangement in said constrained-collapsed position;
e advancing said catheter through a patient's anatomy until said medical device is positioned at said treatment site while both said medical device is in an unexpanded position and said filter arrangement is in said constrained-collapsed position;
f. moving said guide shaft partially or fully off of said filter arrangement to enable said filter arrangement to move to said deployed position;
g. expanding said medical device at said treatment site while said filter arrangement is positioned in said deployed position;
h. moving said guide shaft over at least a portion or all of said filter arrangement to cause said filter arrangement to move to said constrained-collapsed position; and
i. removing said device delivery arrangement from said patient; and
wherein said filter arrangement is configured to capture at least a portion of dislodged emboli that has dislodge due to said expansion of said medical device at said treatment area.

23. The method as defined in claim 22, wherein said catheter includes a balloon and said medical device is positioned at least partially over said balloon.

24. The method as defined in claim 22, wherein said expandable frame is at least partially formed of a) stainless steel, b) cobalt-chromium alloy, c) titanium-aluminum-vanadium alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, m) metal alloy that includes at least 15 atomic weight percent (awt. %) rhenium and/or hafnium, or n) a shape memory material.

25. The method as defined in claim 22, wherein, said expandable frame is at least partially formed of a rhenium and/or hafnium containing alloy.

26. The method as defined in claim 22, wherein expandable frame includes no more than 0.1 wt. % nickel, no more than 0.1 wt. % chromium, and/or no more than 0.1 wt. % cobalt.

27. The method as defined in claim 22, wherein said catheter is a coaxial catheter and said medical device and said filter arrangement are positioned at least partially on or within said coaxial catheter when said medical device is in an unexpanded position.

28. The method as defined in claim 22, wherein an outer surface of at least one component of said medical device includes an enhancement material that has been layered or coated on said outer surface; said enhancement material is formulated to i) provide nitric oxide, and/or ii) promote generation of nitric oxide; said enhancement material is at least partially formulated of oxynitride.

29. The method as defined in claim 22, wherein said medical device includes one or more leaflets, and one or more of an inner skirt, an outer skirt, and/or sutures.

30. A method for manufacturing a medical device system comprising:
a. providing a medical device that include an expandable frame; said expandable frame is formed by the process of:
i. providing metal alloy powder; said metal alloy power has an average particle size of less than 200 mesh; said metal alloy powder is formulated to form a metal alloy of
A) at least 1 wt. % rhenium and/or hafnium and one or more metal alloying additives selected from the group consisting of calcium, carbon, chromium, cobalt, copper, gold, hafnium, iron, magnesium, nickel, niobium, osmium, platinum, rare earth metals, tantalum, technetium, titanium, tungsten, vanadium, yttrium, zinc, and zirconium; and wherein said metal alloy includes 0-2 wt. % of a combination of a) metals other than rhenium and/or hafnium, b) metals other than said one or more said metal alloying additives, c) carbon, d) oxygen, and e) nitrogen; or
B) at least 20 wt. % of primary metal and one or more metal alloying additives, and wherein said primary metal includes one or more metals selected from the group consisting of molybdenum, niobium, tantalum, and tungsten, and wherein said one or more metal alloying additives includes one or more metals selected from the group consisting of calcium, carbon, chromium, cobalt, copper, gold, iron, magnesium, nickel, niobium, osmium, platinum, rare earth metals, silver, tantalum, technetium, titanium, tungsten, vanadium, yttrium, zinc, and zirconium; and wherein the metal alloy includes 0-2 wt. % of a combination of a) metals other than said primary metal, b) metals other than said one or more metal alloying additives, c) carbon, d) oxygen, and e) nitrogen;
ii. consolidating said metal alloy powder into a rod; said step of consolidating includes subjecting said metal alloy powder to an isostatic process that applies a uniform pressure of 400-700MPa from all sides on said metal powder; said step of consolidating occurring in an inert atmosphere, an oxygen reducing atmosphere of hydrogen or an argon and hydrogen mixture, and/or under a vacuum;
iii. sintering said rod of consolidated metal alloy powder;
iv. subjecting said rod to a gun drilling process and/or an EDM cutting process to form a metal alloy tube from said rod; and
V. cutting, etching, grinding, laser cutting, and/or shaving said metal alloy tube to partially or fully form expandable frame; and
b. providing a delivery arrangement; said delivery arrangement includes a delivery system and a filter arrangement; said delivery system includes a catheter and a guide shaft; said catheter and said guide shaft are configured to move independently from one another; at least a portion of said catheter is telescopically received in a cavity of said guide shaft; said expandable frame is positioned on a portion of said catheter; said filter arrangement is connected to a distal region of said catheter such that at least a portion or all of the filter arrangement is located proximal to said medical device when said medical device is releasably positioned on said catheter; said filter arrangement is configured to move between a constrained-collapsed position and a deployed position; said filter arrangement includes a filter frame and a filter material; said filter material is connected to said filter frame; said filter frame is connected to said catheter; said filter frame is formed or a) a plurality of arms that are spaced from another, or b) a plurality of loops that are spaced from one another; said guide shaft is moveable along a longitudinal axis of said catheter and relative to said filter arrangement between a constraining and non-constraining position; said filter arrangement is positioned in said constrained-collapsed position when said guide shaft is in said constraining position due to said guide shaft being positioned partially or fully over said filter arrangement; said filter arrangement is moveable to said deployed position when said guide shaft is in said non-constraining position due to said guide shaft being positioned partially or fully proximal to said filter arrangement.

31. The method as defined in claim 30, wherein said medical device includes one or more leaflets, and one or more of an inner skirt, an outer skirt, and/or sutures.

32. The method as defined in claim 30, wherein said catheter includes a balloon and said medical device is positioned at least partially over said balloon.

33. The method as defined in claim 30, wherein said medical expandable frame is at least partially formed of a metal alloy that includes at least 15 atomic weight percent (awt. %) rhenium and/or hafnium.
